(19) United States
(12) Patent Application Publication
(10) Pub. No.: US 2024/0335373 A1
(43) Pub. Date: Oct. 10, 2024

(54) Methods, Systems and Devices for Post-Fabrication Drug Loading

(71) Applicant: THE UNIVERSITY OF NORTH CAROLINA AT CHAPEL HILL, Chapel Hill, NC (US)

(72) Inventors: Soumya Rahima Benhabbour, Chapel Hill, NC (US); Rima Janusziewicz, Durham, NC (US)

(21) Appl. No.: 18/591,127

(22) Filed: Feb. 29, 2024

Related U.S. Application Data
(62) Division of application No. 18/026,969, filed on Mar. 17, 2023.
(60) Provisional application No. 63/079,953, filed on Sep. 17, 2020.

(51) Int. Cl. A61K 9/00; A61F 6/08; A61K 31/52; A61K 31/565; A61L 31/12; A61L 31/16; A61P 31/18

(52) U.S. Cl. CPC A61K 9/0036; A61F 6/08; A61K 31/52; A61K 31/565; A61L 31/125; A61L 31/16; A61P 31/18; A61L 2300/602

(57) ABSTRACT

A post-fabrication method for drug loading a medical device with an active pharmaceutical ingredient (API). Such medical devices can include a polymer matrix, where the polymer matrix, after exposure to a loading solution with the API, can exhibit a degree of swelling of the polymer matrix and/or a degree of swelling in which the polymer matrix increases in a dimension along an axis. Medical devices including a polymer matrix and an API are provided, where the API is loaded into the polymer matrix by adsorption and/or swelling after fabrication of the polymer matrix, wherein the medical device provides a substantially sustained release of the API for an extended period of time. The medical devices include intravaginal rings (IVR). Methods of treating a subject using the disclosed medical devices are also provided, including treating a subject with an IVR with one or more APIs loaded therein.

### CROSS-REFERENCE TO RELATED APPLICATIONS

This application is a divisional of U.S. patent application Ser. No. 18/026,969 filed on Mar. 17, 2023, which is a 371 of International Application No. PCT/US2021/050949 filed on Sep. 17, 2021, which claims benefit of and priority to U.S. Provisional Patent Application Ser. No. 63/079,953, filed Sep. 17, 2020, the disclosures of which are incorporated herein by reference in their entireties.

### GRANT STATEMENT

This invention was made with government support under Grant Numbers HD100190, AI136002 and AI150358 awarded by the National Institutes of Health. The government has certain rights in the invention.

### TECHNICAL FIELD

The presently disclosed subject matter is directed to the development and optimization of post-fabrication drug loading processes, methods, systems and devices. The presently disclosed subject matter is also directed to medical devices configured with improved release kinetics of pharmaceutical compounds.

### BACKGROUND

Extended-drug delivery systems have long been a part of the overall strategy for controlled therapeutic administration, the primary aim of which is to achieve sustained delivery an active pharmaceutical ingredient (API) within a therapeutically relevant range [1]. These systems provide unique advantages over conventional dosage forms such as oral or direct injection [2] including improved absorption rates, preservation of the API from degradation, and targeted delivery [3]. Polymeric systems in particular allow for tunable therapeutic encapsulation and controlled release through selection of molecular backbone, formation method and drug delivery mechanism [2]. Polymer-based delivery devices can be characterized by their drug release mechanisms as either diffusion-driven, as with silicones and polyurethanes [4], or biodegradable, such as poly (vinyl alcohol) (PVA) or polycaprolactone (PCL) [5]. Appropriate polymer selection is guided by API choice and target indication, which in turn dictates method of device manufacturing [3]. There are numerous device fabrication platforms developed to achieve sustained drug delivery, ranging from electro spun scaffolds [6], in-situ forming implants [7, 8], and hydrogels [9]. Each method has an optimal processing window which may only be suitable for a sub-class of APIs. Therefore, a platform that allows for compatibility to a larger range of drugs, from small molecule hydrophobic to large molecular weight biologics, while maintaining the precision of targeted and sustained release that polymeric devices provide, has long been the target.

The relationship between device geometry and release rate has been well-studied. However, because there is often a relationship between achievable geometry and material, each polymer fabrication or formation is contained within a limited design space. Additive manufacturing (AM), or more commonly 3D printing, is one method of polymer fabrication that enables theoretical design freedom by computationally defining a specific device geometry and replicating through the layerwise assembly or deposition of material [10]. The selective layerwise addition of material can occur through several mechanisms, such as extrusion of a heated filament as with fused deposition modeling (FDM) or with a rastering laser to join polymer or metal particles, as with selective laser sintering (SLS) [11, 12]. AM has been and continues to be explored for its potential toward the design, fabrication, and implementation of polymeric medical devices to achieve greater control and precision of drug delivery [14, 15]. Specifically, the freedom of design principle has been systematically investigated in several AM platforms to tune drug release in a way that could not have been achieved using conventional methods, as the case with designing channeled tablets [16-18], lattice architectures [19, 20], and hydrogels [21].

3D printing, while promising, still presents significant challenges to overcome to achieve application toward medical devices for the sustained and controlled release of medical devices. The layerwise assembly method, across nearly all AM platforms, is not only time-consuming but also imparts directional mechanical strains [22, 23] that can result in part failure and require elaborate methods to overcome [24]. There is a tradeoff between layer thickness, part resolution and total fabrication time, where increased resolution (smaller layer thicknesses), increases the total number of layers and results production times that are prohibitive to manufacturing scale up [25, 26]. Finally, as with conventional methods, there is matter of drug incorporation or encapsulation [13]. Incorporation of API at the point of device fabrication varies by AM method. If melting polymeric material to achieve layerwise assembly, as in the case of FDM or SLS, API incorporation is limited by thermal exposure and solubility just as with injection molding [15]. Polymer materials can be selectively bound as in the case of material jetting or binder jetting methods which unify material through UV exposure or solidification, limiting the potential drug candidates to those that are soluble or not photosensitive [13]. Vat polymerization methods utilize a rastering laser or UV light to selectively cure via free radical polymerization mechanism and is similarly constrained by light exposure and solubility as well as potential degradation in the presence of free radicals [12, 14]. API incorporation post-fabrication is similarly challenging and often results in surface adsorption as opposed to true matrix penetration via absorption.

Digital light synthesis (DLS), also known as Continuous Liquid Interface Production (CLIP), is a novel AM method that utilizes the selective exposure of ultraviolet (UV) light through an oxygen permeable membrane onto a photosensitive resin [27]. DLS offers significant benefits over conventional AM methods such as stereolithography (SL) or fused deposition modeling (FDM) through the comparably fast and layerless fabrication of parts [28]. Compatible resins with the DLS platform include two-part resins containing both a UV-curable component, activated during the 3D-printing step, and a thermally-curable component, activated during a post-fabrication heating step [29], The dual curing allows for the formation of complex polymer matrices which display unique compression and extension properties beyond what is observed with traditional (meth) acrylate chemistries [30]. While translational from a part manufacturing perspective in terms of speed and physical properties [31, 32], DLS is not without its drawbacks when applied to drug delivery, particularly in the case of the dual-cure resins. Incorporation of the API in the resin prior to cure ensures a homogeneous distribution within the part however exposure of UV light and subsequent heat severely restricts the number of compatible APIs that could survive the process without degradation.

Therefore, there is a need for new technologies that utilize efficient and cost effective methods to load medical devices, e.g. intravaginal rings (IVRs), with active pharmaceutical ingredients (APIs). More particularly, there is an unmet need to develop alternative loading routes, systems, methods and techniques for APIs that may not be compatible with the fabrication processes due to thermal or UV-light sensitivity. Moreover, there is a need for streamlined precision controls over loading particularly as it relates to influencing changes in drug release rate from such medical devices. The presently disclosed subject matter addresses these long-felt needs. We present a method to address this by utilizing a post-fabrication absorption process to load a variety of therapeutics into a 3D printed silicone material. Using a controlled system, we systematically investigated the effect of a geometry on the swelling, drug uptake, and drug release in simulated vaginal fluid and correlated these performance parameters to the fundamental characteristics of the part, namely the specific surface area.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided are post-fabrication methods for drug loading a medical device with an active pharmaceutical ingredient (API), the methods comprising providing a medical device comprising a polymer matrix, exposing the medical device to a loading solution comprising the API for a time sufficient to cause the API to be integrated within the polymer matrix, wherein the polymer matrix, after exposure to the loading solution with the API, exhibits a degree of swelling in a range of about 100% to about 1100% of the polymer matrix relative to an unswollen state of the polymer matrix prior to exposure to the solution comprising the API, and/or a degree of swelling in which the polymer matrix increases in a dimension from about 60% to about 500% along an axis. In some embodiments, the medical device comprises an intravaginal ring (IVR), optionally wherein the IVR is 3D printed. The degree of polymer swelling can be influenced by a factor selected from the group consisting of network crosslinking density of the polymer matrix, polymer backbone properties (e.g. MW, charge, polarity), presence of side chains in the polymer matrix, polymer structure (e.g. linear versus branched), dimensions of the medical device (e.g. surface area, volume, thickness), and/or combinations thereof. In some aspects, the degree of polymer swelling is influenced by an interaction of the polymer matrix with a solvent in the solution. In some aspects, a percent solvent uptake and swelling is solvent dependent (i.e. interaction of matrix with solvent).

In some embodiments, a geometry of the medical device influences the degree of swelling and percent drug incorporation/loading, optionally wherein the geometry comprises a part volume defining an amount of macro space within the polymer matrix. In some aspects, a total loaded amount of API is tuned by an initial concentration of the loading solution comprising the API. The role of diffusion distance in drug delivery duration and the role of specific surface area (SSA) in the prediction of drug release can be defined and used to fine-tune drug release. In some aspects, a degree of crosslinking of the polymer matrix is substantially proportional to the degree of swelling, optionally wherein the degree of crosslinking defines an accessibility of a micro space within the polymer matrix. In some embodiments, the degree of swelling is substantially directly proportional to the degree of API loading. At a given degree of swelling of the polymer matrix there can be a substantially linear correlation between API concentration in the loading solution and percent API loaded in the polymer matrix. The degree of swelling of the polymer matrix can substantially increase with increasing diffusion distance in the polymer matrix.

In some aspects, the medical device is exposed to a loading solution comprising more than one API. In some embodiments, the methods further comprise removal of extractables and/or leachables (i.e. unreacted or unincorporated monomers or oligomers) from the polymer matrix of the device.

In some embodiments, API loaded medical devices produced by the disclosed methods are provided. The API loaded medical devices can in some aspects comprise substantially controlled drug release kinetics, optionally wherein the release kinetics can be optimized based on swelling duration, solvent type, API concentration, rate controlling additives, release rate controlling membranes and combinations thereof.

Provided in some aspects are medical devices comprising a polymer matrix and an active pharmaceutical ingredient (API), wherein the API is loaded into the polymer matrix by adsorption and/or swelling after fabrication of the polymer matrix, wherein the medical device is configured to achieve release kinetics in a range of about one day to about 360 days, optionally wherein the release kinetics comprise a substantially sustained release for at least about 30 days or more, optionally for at least about 60 days or more, optionally for at least about 90 days or more, optionally for at least about 120 days or more. The device comprises a geometrical distance and a volume, wherein a release rate of the API from the polymer matrix is controlled by a diffusion distance and a part volume. A release rate of the API from the polymer matrix is controlled by an interaction between the API and polymer matrix, and/or an interaction between the API and a surrounding environment, wherein the surrounding environment comprises one or more of: swelling of polymer matrix parts that impact accessibility to API, a surface area of the device that impacts accessibility to API, another API or release rate controlling additive that impacts accessibility to API, a polymeric membrane that surrounds the device and impacts accessibility to API, and an initial loaded concentration of API and changes thereto as API is released. In some aspects, the medical devices can comprise an intravaginal ring (IVR), wherein the IVR comprises one or more APIs.

Provided in some embodiments are methods of treating a subject, wherein the method of treatment, prevention or diagnostic comprises providing a subject in need of treatment, prevention or a diagnostic, providing a medical device as disclosed herein, and administering, placing and/or applying the medical device to/in the subject in need of treatment, prevention or diagnostic. The medical devices can comprise an intravaginal ring (IVR), wherein the IVR comprises one or more APIs. The medical devices can further comprise a release rate controlling additive. In some aspects, the medical device in such methods can comprise an intravaginal ring (IVR), wherein the IVR comprises a release rate controlling polymeric membrane. The release kinetics of the one or more APIs can be in a range of about one day to about 360 days, and any range in between. In some aspects, the one or more APIs comprises a therapeutic compound selected from an antiviral, antiretroviral, microbicide, contraceptive, antibiotic, hormone, pre-exposure prophylaxis, small molecule drug, macromolecule drug, biopharmaceutical, chemotherapeutic, monoclonal antibody, protein, peptide, diagnostic marker, other pharmaceutical compound, and combinations thereof. In some embodiments, the subject in need of treatment is in need of HIV pre-exposure prophylaxis (PrEP), HIV treatment, contraception, and/or prevention of sexually transmitted diseases (STDs), women's health indication (e.g. infertility, hormone replacement, gynecology oncology, diagnostic). In some embodiments, the subject in need of treatment can be a female human subject or transgender.

These and other objects are achieved in whole or in part by the presently disclosed subject matter. Other objects and advantages of the presently disclosed subject matter will become apparent to those skilled in the art after a study of the following description, Drawings and Examples.

### BRIEF DESCRIPTION OF THE FIGURES

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following figures.

FIGS. 1A-1C are schematic illustrations of the design and fabrication of the system used to systematically investigate the effects of post-fabrication absorption, or post-loading. FIG. 1A shows a computationally-aided design (CAD) of the device, or diffusion block, where the Z (10 mm) and Y (20 mm) dimensions are held constant and the X dimension is varied from about 0.5 to about 7.6 mm. FIG. 1B is a schematic of the digital light synthesis (DLS) fabrication process where the selective display of UV light illuminated through an oxygen permeable window, generating a dead-zone in which polymerization does not occur. Once the oxygen is depleted, the part is formed and pulled from the resin pool. FIG. 1C is a schematic mechanism of SIL 30 resin illustrating the two-step process to achieve a silicone matrix. The dual system is first exposed to UV light via the DLS process and then in a secondary step, exposed to heat in a post-thermal cure to complete the matrix formation.

FIG. 2A is a schematic illustration of a digital micro-mirror device (DMD), and a 4-bit example explanation of how the DMD assigns a pixel state and the effect on total perceived light intensity to the window. FIG. 2B is a schematic illustration of how greyed pixel states are determined using an example slice from the input of a ‘combined’ fabrication.

FIGS. 3A-3E are based on the assessment of DLS fabricated blocks as a function of resin and placement. FIG. 3A is an illustration of combined versus separated block placement on the build platform. Combined placement contained all block types, distinguished by the X dimension distance, with a spacing of 2.5 mm between each block. Separated placement contained a single block type, and thus a single X dimension, with a controlled spacing of 3.75 mm. FIG. 3B is a graph of percent deviation from the input CAD value in the Z dimension (10 mm) as a function of block type and resin. FIG. 3C is a graph of percent deviation from the input CAD value in the Y dimension (20 mm) as a function of block type and resin. FIG. 3D is a graph of the absolute measured distance in the X dimension as a function of block type and resin. FIG. 3E is a graph of percent deviation from the input CAD value in the X dimension (varied) as a function of block type and resin. Error bars represent the standard deviation of n=20 values per condition.

FIGS. 4A-4G show the results of swelling analysis as a function of solvent exposure duration for Acetone (red) and Methanol (blue). All block types (n=3) were immersed in either Ace or MeOH and metrics (mass and azimuthal dimensions) assessed at Day 0, 1, 2, 3, 7, and dried. Degree of swelling was calculated for each block type immersed in (FIG. 4A) Ace or (FIG. 4B) MeOH as a function of exposure duration. Azimuthal dimensions collected were used to calculate surface area for (FIG. 4C) Ace or (FIG. 4D) MeOH and volume (FIG. 4E) Ace or (FIG. 4F) MeOH as a function of exposure duration. Specific surface area (SSA) was computed for all block types in the initial, swollen (Day 1 and 7) and dried states (FIG. 4G). All values represent and average and standard deviation of n=3 blocks per type per condition.

FIGS. 5A-5B show results of Azimuthal swelling and shrinking analysis for a 24 hr. post-loading mimic in Ace and MeOH. FIG. 5A shows the results of assessment of degree of swelling by dimension as a function of block type and solvent. FIG. 5B shows the results of assessment of degree of shrinkage in all dimensions as a function of block type and solvent type. All values represent and average and standard deviation of n=3 blocks per type per condition.

FIGS. 6A-6D are schematic illustrations of experimental designs of data sets utilized in report with diffusion blocks. FIG. 6A shows a method of post-loading with β-estradiol. FIG. 6B shows post-loading mimic (no drug in loading solution) as a function of loading solvent. FIG. 6C shows extraction method, and FIG. 6D shows a release method. Analysis of degree of swelling was conducted using both masses obtained during the loading process as well as dimensions (swollen and dried metrics).

FIG. 7 is a post-loading process flow chart of potential variables and parameters. The flow chart includes part fabrication for purpose of solvent selection based on resin characteristics. Parameters utilized in optimization steps include solvent and loading duration (static and room temperature environment). Parts characterization includes chemical and physical characteristics.

FIG. 8 is a series of images of parts swollen in selected pilot solvents at respective end of immersion time points. The 20 mL scintillation vials did not impede part swelling. It should be noted that the vials curve inward at the top which did impede part removal. The parts were deformed during removal vial tweezers, partly obstructing dimensional measurements.

FIGS. 9A-9B are graphs of the release profiles of β-Estradiol from pre-loaded diffusion blocks. FIG. 9A is a graph of the cumulative release in μg over the course of 28 days. FIG. 9B is a graph of the cumulative release as a percentage of the total loaded over the course of 28 days. Values represent averages and standard deviations of n=4 samples. Resin was quantified to have 10.7±0.5 μg/mg of resin, determined from n=3 samples.

FIGS. 10A-10B are graphs of release profiles of β-Estradiol from post-loaded diffusion blocks as a function of solvent type. FIG. 10A is a graph of the cumulative release in μg over the course of 28 days. FIG. 10B is a graph of cumulative release as a percentage of the total loaded over the course of 28 days. Values represent averages and standard deviations of n=4 samples. Percent cumulative release was quantified utilizing the average values obtained from extraction analysis by respective solvent types.

FIG. 11 is a graphical depiction of release profiles of β-Estradiol from post-loaded diffusion blocks as a function of solvent type compared to the pre-loaded sample set. The average value of the percent cumulative release of the pre-loaded blocks is shown in grey.

FIGS. 12A and 12B are graphs of release profiles of β-Estradiol from post-loaded EstRing sections as a function of solvent type. FIG. 12A is a graph of the cumulative release in μg over the course of 28 days. FIG. 12B is a graph of the cumulative release as a percentage of the total loaded over the course of 28 days. Values represent of n=1 samples. Percent cumulative release was quantified utilizing the average values obtained from extraction analysis by respective solvent types.

FIG. 13 is a schematic illustration outlining successive loading procedure utilizing a single loading solution and diffusion blocks. Diffusion blocks (n=4) were successively loaded into a 20 mL super saturated methanol solution for 24 hr. following optimized post-loading procedures. Blocks were removed and replaced for four cycles yielding n=4 blocks denoted by loading order.

FIGS. 14A-14E include schematics, images and data based on the visualization of the post-loading process with a hydrophobic and hydrophilic dye. FIG. 14A is a schematic of the visualization process, where Xᵢ=4.0 mm. DLS SIL 30 blocks along with placebo sections of EstRing (IM silicone) and Nuvaring (IM EVA) are shown in FIG. 14B, with an image of sections immersed in an RhB/MeOH solution for 24 hr. and removed in FIG. 14C. FIG. 14D is an image of 4.0 mm block types (n=3 per condition) that were immersed in a series of hydrophobic dye solutions (RhB), hydrophilic dye solutions (NBA) and combination (RhB+NBA) for 24 hrs, removed and bisected. The degree of swelling (mass and azimuth) were calculated as a function of dye type and concentration as well as matrix fraction, as shown in FIG. 14E.

FIGS. 15A-15D are images showing post-loading with RhB as a function of material type. Three materials were selected: EstRing, NuvaRing, and SIL 30 Block. FIG. 15A is an image of material sections prior to post-loading. All samples have comparable mass. FIG. 15B is an image of representative samples during the post-loading process in 20 mL of 80 μg/mL RhB in MeOH. FIG. 15C is an image of representative samples following removal from post-loading solution. Dashed line on the SIL 30 block indicates cross-section. The ring sections were bisected. FIG. 15D is a cross-sectional image of SIL 30 block following removal from post-loading indicating complete and even penetration of RhB.

FIG. 16 is an image of bisected EstRing sections following post-loading. All three samples are shown, identified by number. There are visual differences between the samples in terms of RhB uptake which may partly explain the large variability observed in the degree of swelling, which is a mass-based calculation.

FIG. 17 includes images of post-loading as a function of RhB concentration in MeOH. SIL 30 blocks (4 mm) were post-loaded in a four-fold dilution series of RhB in methanol at 40, 10, and 2.5 μg/mL. Representative samples are shown during the post-loading process, immediately after removal from solution, and during the extraction process.

FIGS. 18A and 18B are graphs showing the quantification of RhB extraction of post-loaded SIL 30 blocks as a function of loading solution concentration. Extraction was conducted in 20 mL of EtOH for 72 hrs, as previously described. Quantification was conducted using fluorescence detection on a plate reader. Both the calibration curve, prepared in EtOH, and samples were measured in triplicate. Each concentration contained n=3 samples therefore each point represents an average of n=9 points. FIG. 18A shows total RhB extracted from blocks as a function of loading concentration. FIG. 18B shows weight percent loading as a function loading concentration where the dry mass following post-loading was used.

FIG. 19 includes images of the fabrication of diffusion blocks as a function of distance. Image of CAD in the Carbon UI and image of blocks fabricated in SIL 30.

FIG. 20 is a schematic of an experimental design to evaluate the effect of diffusion distance and solvent on swelling behavior. Simulation of post-loading process for blocks as a function of distance with two solvents, methanol and acetone, without the presence of an API.

FIG. 21 includes graphical depictions of swelling behavior as a function of diffusion distance and solvent type. Degree of swelling was calculated for each azimuthal axis, as previously defined, as a function of diffusion distance. The swollen and dried dimensions were used for this calculation as a function of solvent type. Average and standard deviations represent n=3 samples per sample condition.

FIGS. 22A and 22B are graphical depictions of gel fraction (FIG. 22A) and degree of swelling (FIG. 22B) as a function of diffusion distance and solvent type. Gel fractions and degrees of swelling were calculated using the masses of the blocks. Average and standard deviations represent n=3 for each sample condition.

FIGS. 23A-23D show the comparison of swelling behavior between unloaded and drug-loaded blocks as a function of diffusion distance in the X direction (FIG. 23A), Y direction (FIG. 23B), Z direction (FIG. 23C) and of mass (FIG. 23D). Swelling dimensions and masses are compared for samples swollen in methanol. Average and standard deviations represent n=3 samples per loading condition and distance.

FIGS. 24A-24C show comparisons of pre-loaded and post-loaded blocks as a function of diffusion distance. Initial metrics for the pre-loaded (FIG. 24A) and unloaded (FIG. 24B; to be post-loaded) blocks. Directions are indicated on the CAD model of the diffusion block. Percent deviation in Z was calculated relative to the specified distance in CAD (FIG. 24C). Negative deviations represent distances smaller than CAD whereas positive represent distances larger than CAD. Average and standard deviations represent n=4 samples per condition.

FIGS. 25A and 25B include graphical depictions of weight percent loading by diffusion distance. FIG. 25A is a comparison of weight percent of β-estradiol extracted from post-loaded parts from two different loading rounds. FIG. 25B is a comparison of weight percent of β-estradiol extracted from pre-loaded versus post-loaded structures. Average and standard deviations represent n=3 samples per condition.

FIG. 26 is a schematic of a proposed explanation for pre-loading weight percent deviation. Pre-loaded structures fabricated n=9 per print with intentional 1.0 wt. % loading versus pre-loaded structures fabricated n=80 per print with intentional 4.0 wt. % loading. Loading deviation is attributed to ‘crowding’ in which the large number of parts per prints disrupts the two-component balance and alters the solubility of the drug in the resin.

FIGS. 27A-27F are graphs based on data from solvent-treated block swelling analysis in simulated vaginal fluid (SVF) at 37° C. as a function of time. FIG. 27A is for experimentally obtained mass (mg). FIG. 27B is for calculated volume (mm³) from collected X, Y, and Z dimensions. FIG. 27C is for calculated density (mg/mm³). Percent increase from day 0 to day 28 in the block mass and block diffusion distance (X) is shown in FIG. 27D. Percent increase in the calculated block volume and surface area from day 0 to day 28 as shown in FIG. 27E. Computed specific surface area (SSA, mm⁻¹) and resulting equations logarithmic curves fitted for each block type with associated coefficient of determination as shown in FIG. 27F. Experimentally obtained values are represented as average and standard deviation of n=3 samples per condition; calculated values are represented as average and computed propagation of error of n=3 samples per condition.

FIG. 28 is a comparison of total loading (mg) of pre-loaded and post-loaded diffusion blocks as a function of distance. Total loading values were determined from extraction by EtOH. Average and standard deviation values represent n=4 samples per condition. X-axis error was determined from n=8 samples per loading condition.

FIG. 29 includes representative release chromatograms as a function of loading type and two distances. Leachable presence is highlight in green and absence in red as a function of loading type and diffusion distance.

FIGS. 30A and 30B are graphical depictions of the cumulative release of β-estradiol from pre-loaded blocks as a function of distance and release day. Release was conducted in simulated vaginal fluid and values represent average and standard deviations of n=4 samples per condition. FIG. 30A shows cumulative release by percent. FIG. 30B shows cumulative release by percent compared to previously obtained results.

FIGS. 31A and 31B are graphical depictions of the cumulative release of β-estradiol from post-loaded blocks as a function of distance and release day. Release was conducted in simulated vaginal fluid and values represent average and standard deviations of n=4 samples per condition. FIG. 31A shows cumulative release by percent. FIG. 31B shows cumulative release by percent compared to previously obtained results.

FIGS. 32A-32F are graphs based on the analysis of model drug loading in SIL 30 blocks. FIG. 32A is for total loading (mg) of β-Est (red) and FdA (blue) per block. FIG. 32B is for loading by weight percent (%) of β-Est (red) and FdA (blue) per block. FIG. 32C is for calculation of specific surface area (SSA) in the initial and swollen states for β-Est and FdA. FIG. 32D is for calculation of the difference (delta) in swollen from initial SSA for both model drugs. FIG. 32E is for normalization of weight percent loading by swollen specific surface area (S-SSA). FIG. 32F is based on normalization of weight percent loading by Delta SSA. X-axis values represent average and standard deviation of block distances in the X dimension, n=4 per distance. Y-axis values represent average and standard deviation for FIGS. 32A and 32B compounded error for FIGS. 32C-32F.

FIGS. 33A-33H are graphical depictions of data from in vitro release of blocks loaded with model drugs β-Est (FIGS. 33A, 33C, 33E, 33G) and FdA (FIGS. 33B, 33D, 33F, 33H) in SVF as a function of diffusion distance. Cumulative microgram (μg) release into SVF of (FIG. 33A) β-est and (FIG. 33B) FdA. Cumulative percent (%) release into SVF of (FIG. 33C) β-est and (FIG. 33D) FdA. Cumulative microgram (μg) release normalized by placebo swelling in SVF (μg/mm⁻¹) of (FIG. 33E) β-est and (FIG. 33F) FdA. Cumulative percent release normalized by placebo swelling in SVF (%/mm⁻¹) of (FIG. 33G) β-est and (FIG. 33H) FdA. Values represent average and standard deviation of n=4 samples per block type for FIG. 33A-D, and average and propagated error of n=4 samples per block type for FIG. 33E-H.

FIGS. 34A-34D show results of the analysis of burst release of model drugs from SIL 30 blocks as a function of distance. Burst release by micrograms and percentage for (FIG. 34A) β-Est and (FIG. 34B) FdA. Microgram and percent normalized burst release by placebo SSA swelling in SVF for (FIG. 34C) β-Est and (FIG. 34D) FdA.

FIGS. 35A-35D show results of release of EFdA in SVF from 6.0 and 7.6 mm block types. Cumulative microgram (FIG. 35A) and percent (FIG. 35B) release as a function of time. Microgram (FIG. 35C) and percent (FIG. 35D) release normalized by placebo SSA in SVF. (FIG. 35E) Tabulation of release values. Drug loading amount and weight percent. Burst release quantified as the cumulative release in the first 24 hrs. Zero order release determined from linear regression beginning at Day 2. All values represent average and standard deviation of n=4 samples per block type.

FIGS. 36A-36E include schematics and data pertaining to the translation of post-loading process into geometrically complex IVRs with model drug. FIG. 36A shows the theoretical values of four IVR designs, noted by unit cell used. In vitro release into SVF. FIG. 36B includes CAD models of unit cell design and stereo microscopy imaging of fabricated IVRs in SIL 30 with (FIG. 36C) cumulative microgram release per day by design and (FIG. 36D) cumulative percent release per day by design. FIG. 36E summarizes tabulation of release profile characteristics. Values represent an average and standard deviation n=4 samples per design.

FIG. 37 is a schematic of SIL30 3D CLIP Fabrication and the process of preloading and post-loading β-estradiol on the IVRs.

FIGS. 38A-38C include in vitro release profiles of β-estradiol release from SIL30 3D CLIP printed IVRs prepared by preload and post-load methods of API incorporation. IVRs were incubated at 37° C. in SVF media and data collected over 35 days. Error bars represent standard deviation of n=3 samples. FIG. 38A shows the total cumulative amount (μg) of β-estradiol released from the IVRs. FIG. 38B shows the percent cumulative release of β-estradiol the IVRs. FIG. 38C shows the reload and postload comparison of release kinetics of β-estradiol.

FIGS. 39A-39C include in vitro release profiles of β-estradiol from different designs using post-load as method of API incorporation. IVRs were incubated at 37° C. in SVF media and data collected over 35 days. Error bars represent standard deviation of n=3 samples. FIG. 39A shows the total cumulative amount (μg) of β-estradiol released from the IVRs. FIG. 39B shows the percent cumulative release of β-estradiol the IVRs. FIG. 39C shows the comparison of release kinetics of β-estradiol from post-loaded IVRs with different designs.

FIG. 40A shows selected APIs for MPT IVR and their target loading and daily release rate, with FIG. 40B showing the linear loading equations developed and validate for each API by HPLC analysis.

FIG. 41 includes a comparison of single, dual and triple drug IVRs. Top panel: Percent release kinetics of pritelivir (anti-HSV-2, PTV), dapivirine (anti-HIV, DPV) and levonorgestrel (contraceptive, LNG): Bottom panel: Amount of API (μg) released over time.

FIG. 42 provides data from in vitro release studies of EFdA/EE/ENG IVRs. IVRs (n=3) were loaded with EFdA/EE/ENG and incubated in SVF (pH 4.2) at 37 C. Drug release was quantified by HPLC analysis.

FIGS. 43A and 43B provide data from in vivo PK studies of EFdA-loaded IVRs in pigtailed macaques. Plasma and PBMC levels of EFdA and EFdAtp respectively for (FIG. 43A) a 45 mg EFdA IVR, and (FIG. 43B) a 62 mg EFdA IVR.

FIGS. 44A and 44B provide in vivo PK studies of EFdA-loaded IVRs in pigtailed macaques. Vaginal tissue levels of EFdA collected at proximal (P) and distal (D) sites relative to IVR placement for (FIG. 44A) a 45 mg EFdA IVR, and (FIG. 44B) a 62 mg EFdA IVR.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

The terminology used herein is for the purpose of describing particular embodiments only and is not intended to be limiting of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a unit cell” includes a plurality of such unit cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the term “subject” refers to an individual (e.g., human, animal, or other organism) to be assessed, evaluated, and/or treated by the methods, devices, systems or compositions of the presently disclosed subject matter. Subjects include, but are not limited to, mammals (e.g., murines, simians, equines, bovines, porcines, canines, felines, and the like), and includes humans. As used herein, the terms “subject” and “patient” are used interchangeably, unless otherwise noted.

As used herein, the terms “effective amount” and “therapeutically effective amount” are used interchangeably and refer to the amount that provides a therapeutic effect, e.g., an amount of a composition or active pharmaceutical ingredient that is effective to treat or prevent pathological conditions in a subject.

As used herein, the term “adjuvant” as used herein refers to an agent which enhances the pharmaceutical effect of another agent.

The terms “compound” and “active pharmaceutical ingredient” can be used interchangeably, and as used herein, refer to any type of substance or agent that is commonly considered a chemical, drug, or a candidate for use as a drug, pharmaceutical, therapeutic agent, and the like, as well as combinations and mixtures of the above.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “modulate”, as used herein, refers to changing the level of an activity, function, or process. The term “modulate” encompasses both inhibiting and stimulating an activity, function, or process.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

### Overview of the Presently Disclosed Subject Matter

Provided herein are post-fabrication methods for drug loading a medical device with an active pharmaceutical ingredient (API), or other desired drug compound or therapeutic composition. Such methods can include providing a medical device comprising a polymer matrix, exposing the medical device to a solution comprising the API for a time sufficient to cause the API to be integrated within the polymer matrix, wherein the polymer matrix, after exposure to the solution with the API, exhibits a degree of swelling in a range of about 100% to about 1100% of the original polymer matrix, and/or a degree of swelling in which an object increases in dimension from about 60% to about 500% along an axis. The medical device can comprise an intravaginal ring (IVR), optionally wherein the IVR is 3D printed or otherwise additively manufactured.

In such methods the degree of polymer swelling can be influenced by a factor selected from the group consisting of network crosslinking density, polymer backbone properties (e.g. molecular weight (MW), charge, polarity), presence of side chains, polymer structure (e.g. linear vs. branched), device dimensions (e.g. surface area, volume, thickness), and/or combinations thereof. In some aspects, the degree of polymer swelling can be influenced by an interaction of the polymer matrix with a solvent in the solution. Sometimes a percent solvent uptake and swelling is solvent dependent (i.e. interaction of matrix with solvent).

In some embodiments the geometry of the medical device can influence the degree of swelling and percent drug incorporation/loading, optionally wherein the geometry comprises a part volume defining an amount of macro space within the polymer matrix. A degree of crosslinking of the polymer matrix can be substantially proportional to the degree of swelling, optionally wherein the degree of crosslinking defines an accessibility of a micro space within the polymer matrix. The degree of swelling can be substantially directly proportional to the degree of API loading. At a given degree of swelling of the polymer matrix within the specified range there is a substantially linear correlation between API concentration in the loading solution and percent API loaded in the polymer matrix.

In some embodiments, the medical device is exposed to a solution comprising more than one APIs. In some aspects, such methods further comprise removal of extractables/leachables (i.e. unreacted or unincorporated monomers or oligomers) from the polymer matrix of the device.

Also provided herein are API loaded medical devices produced by the methods disclosed herein. The API loaded medical devices can comprise substantially controlled drug release kinetics, optionally wherein the release kinetics can be enhanced or optimized based on swelling duration, solvent type, API concentration, rate controlling additives, release rate controlling membranes and combinations thereof.

Also provided herein are medical devices comprising a polymer matrix and an API, wherein the API is loaded into the polymer matrix after fabrication of the polymer matrix by adsorption and/or swelling, wherein the medical device is configured to achieve release kinetics in a range of about one day to about 360 days. Such devices can comprise a geometrical distance and a volume, wherein a release rate of the API from the polymer matrix is controlled by a diffusion distance and a part volume. In some embodiments, a release rate of the API from the polymer matrix is controlled by an interaction between the API and polymer matrix, and/or an interaction between the API and a surrounding environment, wherein the surrounding environment comprises one or more of: swelling of polymer matrix parts that impact accessibility to API; a surface area of the device that impacts accessibility to API; another API or release rate controlling additive that impacts accessibility to API; a polymeric membrane that surrounds the device and impacts accessibility to API; and an initial loaded concentration of API and changes thereto as API is released. Such medical devices can comprise, for example, an intravaginal ring (IVR), wherein the IVR comprises one or more APIs.

Finally, provided herein in some embodiments are methods of treating a subject, wherein the method of treatment, prevention or diagnostic can comprise providing a subject in need of treatment, prevention or a diagnostic, providing a medical device of any of the above claims, and administering, placing and/or applying the medical device to/in the subject in need of treatment, prevention or diagnostic. The medical device in such methods can in some embodiments comprise an IVR, wherein the IVR comprises one or more APIs. The medical device in such methods can further comprise a release rate controlling additive. The medical device in such methods can comprise an IVR, wherein the IVR comprises a release rate controlling polymeric membrane. Release kinetics of the one or more APIs are in a range of about one day to about 360 days, about 15 days to about 360 days, about 30 days to about 360 days, about 100 days to about 300 days, greater than about 30 days, greater than about 60 days, greater than about 90 days, greater than about 120 days, greater than about 200 days, greater than about 300 days, or greater than about 360 days or more. The one or more APIs can comprise a therapeutic compound selected from an antiviral, antiretroviral, microbicide, contraceptive, antibiotic, hormone, pre-exposure prophylaxis, small molecule drug, macromolecule drug, biopharmaceutical, chemotherapeutic, monoclonal antibody, protein, peptide, diagnostic marker, other pharmaceutical compound, and combinations thereof. In such methods the subject in need of treatment can be in need of HIV pre-exposure prophylaxis (PrEP), HIV treatment, contraception, and/or prevention of sexually transmitted diseases (STDs), women's health indication (e.g. infertility, hormone replacement, gynecology oncology, diagnostic). In some aspects, the subject in need of treatment can be a female human subject or transgender.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Materials and Methods

### Computationally-Aided Design (CAD)

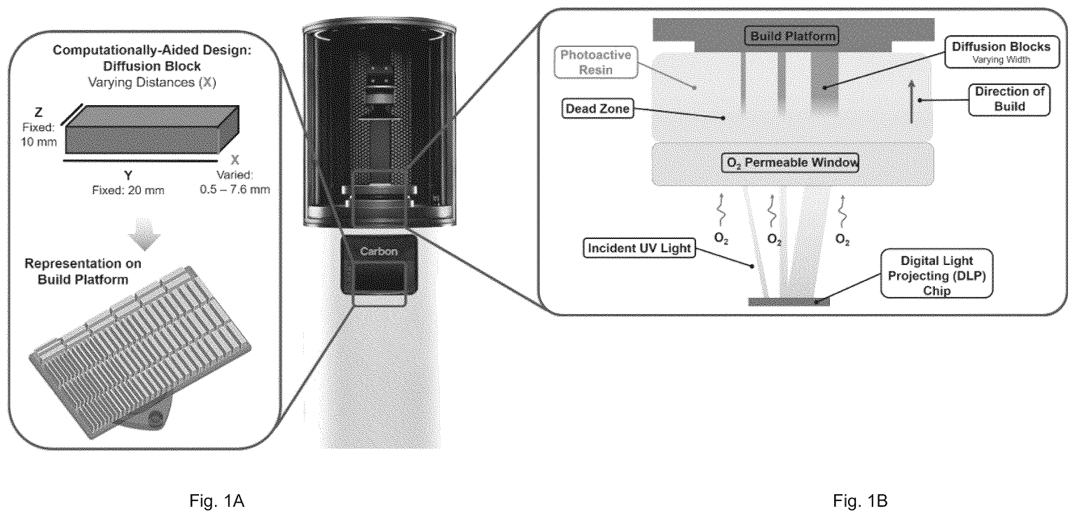

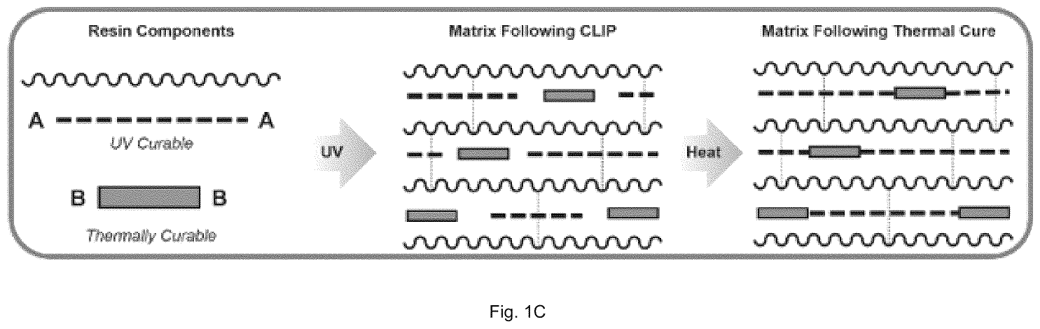

Diffusion Blocks. Diffusion blocks were defined as prismatic rectangles of specified length (Z, constant), width (Y, constant), and height (X, variable). Blocks were defined by the X (variable) dimension. Blocks were generated in SolidWorks (Dassault Systèmes). As described in FIG. 1, the Z and Y dimensions were held constant at 10 and 20 mm, respectively. The X dimension was set at 0.5, 1.0, 2.0, 3.0, 4.0, 6.0, and 7.6 mm to yield seven unique block types. All designs were converted into standard tessellation language (.STL, binary) and exported. Once uploaded onto the Carbon user interface, each block was embossed using labeling software to generate a unique tag denoting block distance designation and number in set. These tags were determined not to interfere with subsequent swelling and release testing.

Unit Cells. All unit cell used were generated in SolidWorks (Dassault Systèmes). The cylinder unit cell was generated to have a 5:4 outer to inner diameter ratio. The Honeycomb, Trident, and Diamond unit cells were generated to have internally consistent strut thicknesses. All unit cells were arrayed to yield approximately 0.5 mm struts when fabricated in SIL 30 using Digital Light Synthesis (DLS). As such, the unit cell sizes (defined as a cube of X, Y, and Z) were set as follows: cylinder, trident and diamond at 3.80×3.80×3.80 mm and honeycomb at 2.53×2.53×2.53 mm. These values represent integer distances within the 7.60 mm cross-section of the IVR (3.80 mm is 1:2 and 2.53 mm is 1:3)

Geometrically Complex IVRs. Geometrically complex intravaginal rings (IVR) designs were generated using methods as disclosed in PCT International Application Serial No. PCT/US2017/023777 (published as WO 2017/165624), the entirety of which is incorporated herein by reference. Briefly, the multistep process begins with a ‘template ring’ of given outer diameter (54 mm) and cross-sectional diameter (7.6 mm) generated in SolidWorks (Dassault Systèmes) and converted to a .STL. The template was imported into Magics (Materialise) and a selected unit cell arrayed into the template using the ‘Scaffold’ feature. Rings were exported as a .STL. Separately, a ring band was generated in SolidWorks (Dassault Systèmes) to encase the ring with dimensions of 4.0 mm height and 0.6 mm thickness and exported as a .STL. Both the geometrically complex IVR and band were imported into MeshMixer (AutoDesk), cenetered on the absolutely origin, combined and exported as a unified .STL. Rings were imported into Magics to correct any tessellation errors accumulated during the file transfer processes. Fully banded geometrically complex rings were exported as a .STL for fabrication.

### Fabrication of Diffusion Blocks With Digital Light Synthesis (DLS)

Printer. Parts were fabricated with an M1 DLS (Digital Light Synthesis) printer (Carbon, Inc.). Diffusion blocks were fabricated with the XY plane of the block oriented to the build platform. Geometrically complex IVRs were fabricated vertically and manually supported using the support feature within the Carbon user interface. Each ring was supported using approximately 40 supports covering the bottom third of the IVR closest to the build platform.

Blocks in SIL 30. Diffusion blocks were fabricated in SIL 30 using two methods: ‘combined’ and ‘separated’. Using the same project as the prototyped blocks, ‘combined’ batches were fabricated n=20 of each type spaced 2.5 mm apart. Batches fabricated as ‘separated’ consisted of each individual block type, replicated n=40, and spaced 3.75 mm apart. This distance was selected to represent an integer value of the projected pixel of the DLS printer (75 μm). Approximately 100 mL of SIL 30 was dispensed from a static mixer into the build reservoir. Each print was 34 min. long using standard slicing of 100 μm.

### Post-Fabrication Treatment

Blocks in Prototyping Resin. Upon completion of the print, blocks fabricated in UMA prototyping resin were removed from the build platform and placed in a sealed container with 200 mL of isopropyl alcohol (IPA). The container was placed on a shaker table for 5 min. The container was removed and using tweezers, the solvent stirred to ensure no blocks were adhered to the container or each other. Blocks were then removed from the solvent and air-dried for 1 hr. Parts were treated to a UV post-cure with a FireJet Fj800 Controller (Phaseon Technology) in a chamber purged with N₂ for 30 s prior to a 2 min exposure of 20 mW/cm² 385 nm light per side.

Blocks in SIL 30. Blocks fabricated in SIL 30 were treated to a post-fabrication cleaning procedure modified from the guidance provided by Carbon. Blocks were removed from the build platform with a razor and placed into a sealed container with 300 mL isopropyl alcohol (IPA). The container was placed on a shaker table from 1 minute then the blocks were removed. Blocks were laid individually on WipeAll towels to ensure no blocks were adhered to one another and allowed to air-dry for approximately 1 hr. Parts were then placed in a programmable oven to initiate a secondary thermal post cure. The program followed recommended curing, beginning at 31° C. and ramping up to 120° C. over 15 min, holding at 120° C. for 8 hr and finishing by ramping down to 31° C. in 15 min. Parts were removed from the oven for immediate further testing or stored at −4° C.

Geometrically Complex IVRs in SIL 30. IVRs fabricated in SIL 30 were treated to a post-fabrication cleaning procedure modified from the guidance provided by Carbon, as previously described. Briefly, parts were removed from the build platform, the supports removed, and the rings placed in a sealed container with 200 mL of IPA. The container was placed on a shaker table for 30 s and then the rings were removed from the solvent. The band of each ring was smoothed using a razor and all rings were placed in a manual spinner to remove excess solvent for 1 min. Rings were then pressed between two Teflon plates from approximately 45 min. The washing and spinning steps were repeated. Finally, parts were placed in a programmable oven to initiate a secondary thermal post cure. The program followed recommended curing, beginning at 31° C. and ramping up to 120° C. over 15 min, holding at 120° C. for 8 hr and finishing by ramping down to 31° C. in 15 min. Parts were removed from the oven for immediate further testing or stored at −4° C.

### Assessment of Diffusion Block Dimensional Accuracy

Block Fabrication. All block types were fabricated in prototyping resin (UMA) under combined conditions (n=3 for each distance) as well as in silicone resin (SIL 30) under combined and separate conditions (n=3 for each distance).

Dimensional Analysis. Azimuthal axis dimensions were taken for each block as a function of type, resin, and fabrication condition.

Dimensional Accuracy. Percent deviation from CAD was calculated using the following equation: % Deviation from CAD=100×(D_{Exp}−D_{CAD})/D_{CAD} where D_{Exp} is the experimentally determined distance (X, Y, or Z) and D_{CAD} was the input distance in the CAD file. All values were calculated individually for each block and reported as average and standard deviation.

### Diffusion Block Metrics for Placebo Testing

Blocks fabricated in SIL 30 were assessed for swelling compatibility under placebo loading conditions in methanol and acetone. Placebo testing was conducted under conditions mimicking the post-loading process and under conditions to completely remove the soluble fraction within the SIL 30 matrix. In all processes, the metrics of mass and distance in the X, Y, and Z azimuthal dimensions were obtained during the initial, swollen and dried stages. Distance values were obtained using calipers. For each treatment and block type n=3 blocks were used and average and standard deviation values determined.

Swell Testing as a Function of Time. Initial metrics for blocks (n=3) of each type were taken and then blocks were batch immersed in either acetone or methanol. Swelling metrics were taken at days 1, 2, 3, and 7. Following Day 7 assessment, blocks were dried for approximately 48 hrs. and dried metrics were collected. Blocks were tracked individually throughout the process based on unique tag.

Post-Loading Mimic. The post-loading process was mimicked using either methanol or acetone in which blocks (n=3 of each type) were immersed in 30 mL of solvent for 24 hr., removed, and air-dried for approximately 48 hrs. Metrics were collected in the initial, swollen, and dried state.

Gel Fraction. The complete soluble fraction removal was conducted in either methanol or acetone in which a specified block type was immersed individually in 30 mL of solvent for 7-days, removed, and air-dried for approximately 48 hrs.

Calculations. Degree of swelling was calculated as a function of time using the following equation: Degree of Swelling (%)=100×(M_{S}−M_{I})/M_{I} where Mₛ is the mass in the swollen state and M_{I} was the mass in the initial state. Surface area and volume were experimentally determined in the initial, swollen (Day 1, 2, 3, and 7), and dried states using standard formulas for rectangles. Specific surface area (SSA) was calculated as:

$$Specific Surface Area ( {mm}^{- 1 )} = Surface Area ( {mm}^{2} ) / Volume ( {mm}^{3} ) .$$

Degree of swelling (%) for each azimuthal axis was calculated as: Degree of Swelling (%)=100×(D_{S}−D_{I})/D_{I} where D_{S} is the swollen dimension and D_{I} is the initial dimension. Degree of shrinkage for each azimuthal axis was calculated as: Degree of Swelling (%)=100×(D_{F}−D_{I})/D_{I} where D_{F}is the final dimension and D_{I} is the initial dimension. The matrix fraction, that is the fraction of both the residual soluble and insoluble material remaining in the matrix, was calculated using the following equation: Matrix Fraction=M_{D-24 Hr.}/M_{I} where M_{D-24 Hr.} is the mass of the dried block following immersion in solvent for 24 hr. and M_{I} is the initial mass. Gel fraction was calculated as: Gel Fraction=M_{D-7 Day.}/M_{I} where M_{D-7 Days} is the mass of the dried block following immersion in solvent for 7 days. All values were calculated individually for each block and reported as average and standard deviation.

$$Normalized Sewlling = Degree of Swelling ( \% ) / Part SSA ( {mm}^{- 1} )$$

### Post-Loading Visualization

The post-loading absorption process was tested with both hydrophobic and hydrophilic dyes. A hydrophobic solution was prepared with rhodamine B (RhB, Sigma Aldrich) in methanol at a concentration of 0.08 mg/mL. This stock solution was serial diluted to obtained solutions at 0.04, 0.02, 0.01, and 0.005 mg/mL. A similar method was used to obtain a hydrophilic solution set containing Nile Blue A (NBA, Sigma Aldrich) in methanol. A stock solution of both dyes was prepared in methanol with 0.04 mg/mL RhB and 0.04 mg/mL NBA and serial diluted to obtain solutions at individual dye concentrations of 0.02, 0.01, 0.005, and 0.0025 mg/mL, with the total dye loading held constant.

### Swelling Analysis of Placebo Blocks in SVF

Block Preparation and Swelling in Simulated Vaginal Fluid (SVF). Blocks (n=3 of each type) were fabricated in SIL 30 as previously described and treated to a 24 hr post-loading mimic cycle in acetone. Placebo blocks were assessed for initial metrics and batch immersed in jar containing 400 mL simulated vaginal fluid (SVF) and placed in an incubator at 37° C. The SVF consisted of 25 mM sodium acetate buffer (pH 4.2) plus 2% Solutol (Kolliphor HS 15). Blocks were assessed for metrics at the following time points: day 1, 2, 3, 4, 7, 8, 9, 10, 11, 14, 21 and 28.

Calculations. Block metrics were used to calculate volume, surface area, density, mass increase (%), increase in the z-axis (%), volume increase (%), surface area increase (%) and specific surface area (SSA). Plots were generated as either a function of time or a function of block distance and values reported as an average and standard deviation. SSA plots were generated as a function of time for each block type, a log curve fitted and an equation and coefficient of variation determined.

Post-Loading with Model Drugs

Loading. Two small-molecule model drugs were selected to investigate the post-loading absorption method: β-estradiol (hydrophobic, log p=3.75) and 2-fluoro-2′-deoxyadenosine (FdA) (hydrophilic, log p=−0.57). Solutions of 5 mg/mL of drug in acetone were prepared and validated via HPLC. Blocks (n=8 of each type) were batch immersed for 24 hr, removed and air dried for approximately 48 hrs. Blocks were then divided into an extraction set and a release set.

Extraction. Drug-loaded blocks were extracted using methods previously described in Section 2.6 using acetone as the extraction solvent. Aliquots were collected and analyzed with HPLC. Weight percent loading was determined as the total drug extracted relative to the mass of the dried blocks.

Release in SVF. Post-loaded blocks were placed individually into sealed jars containing 60 mL of simulated vaginal fluid (SVF). The SVF consisted of 25 mM sodium acetate buffer (pH 4.2) plus 2% Solutol (Kolliphor HS 15). Jars were placed in incubator at 37° C. Aliquots of 1 mL were taken daily and replaced with fresh media. Sink conditions were maintained and monitored with media changes occurring when saturation had been reached. The saturation solubility of β-estradiol in SVF was determined to be 108 μg/mL The concentration of β-estradiol in the aliquots was determined using an Agilent 1260 HPLC with a Diode Array Detector, on an Inertsil ODS-3 column (4.6×150 mm, 5 um) maintained at 40° C., with a flow rate of 1.0 mL/min, 25 μl sample injection, and an acetonitrile/water mobile phase, each modified with 0.1% trifluoroacetic acid. A gradient method was utilized to achieve separation (0-20 min: 5%-100% acetonitrile; 20-22 min: 100% acetonitrile; 23-25 min: 5% acetonitrile). β-Estradiol was eluted at 13.8 min and measured at 280 nm. FdA was eluated at 5.3 min and measured at 265 nm. Area under the curve (AUC) was computed using Chemstation software, and concentrations were derived from a calibration curve generated using β-Estradiol or FdA standards prepared in 100% acetonitrile (250 ug/ml-61 ng/ml). Release was determined as complete when additional drug was no longer detected in the SVF medium. Blocks were then removed and placed in acetone for extraction of residual or trapped drug. Aliquots were analyzed via HPLC.

Calculations. Release of model drugs were reported as either cumulative amount (μg) or percent (%) over time. Burst release was determined as the cumulative amount (μg) or percent (%) of drug released within 24 hr. The release of drug over time was plotted for each block and linear curves fitted from Day 2 onward to obtain equations describing zero order release and associated coefficients of variation. Values were averaged for each block type and reported as average and standard deviation. To account for changes in block geometry associated with SVF swelling, the SSA equations determined in Section 2.7 were used to calculate specific surface areas (mm⁻¹) for each block using the initial dimensions of the release blocks. Calculated values were then used to normalize the release data by computing either μg/mm⁻¹ or %/mm⁻1 as a function of release duration. Burst release values were similarly normalized using the Day 1 SSA values obtained during placebo swelling analysis in SVF (Section 2.7).

### Post-Loading With Target Therapeutic API

Block types 6.0 and 7.6 mm (n=8 of each type) were fabricated in SIL 30. A solution containing 8.562 mg/mL of 4′-ethynyl-2-fluoro-2′-deoxyadenosine (EFdA) in acetone was prepared and validated via HPLC. Blocks were batch immersed in post-loading solution for up to 24 hr. Blocks were divided into extraction (n=4) and release (n=4) sets. EFdA was extracted from blocks in acetone as previously described and quantified with HPLC. Release blocks were individually immersed in 60 mL of SVF and placed in an incubator at 37° C. Aliquots and analysis were completed using methods previously described. EFdA was quantified with HPLC, eluting at 5.31 min at 265 nm. Concentrations were derived from a calibration curve generated using EFdA prepared in 100% acetonitrile (250 ug/ml-61 ng/ml) with the LOQ determined as 0.21 μg/mL. Values were reported as cumulative release amount (μg) or percent (%) as a function of time and normalized by SSA as previously described for the model drugs. All values reported represent average and standard deviation of n=4 samples.

### Post-Loading Geometrically Complex IVRs

Loading. Geometrically complex IVRs with varying unit cells but constant strut thickness were fabricated in SIL 30 and cleaned as described in Section 2.3 and 2.4, respectively (n=5 per ring design). Rings were assessed for metrics of mass, outer diameter (mm), and cross-sectional diameter (mm) and batch immersed in a solution of 22 mg/mL β-Estradiol in methanol for 24 hrs. Rings were removed and air dried for approximately 48 hrs.

Extraction. One ring of each design was designated for extraction using methods previously described. A representative weight percent loading for each design was determined.

Release in SVF. Rings were individually placed in jars with 60 mL SVF in an incubator at 37° C. Aliquots of 1 mL were taken and analyzed via HPLC as previously described. Rings were removed at Day 28 and underwent a full extraction to determine total loading. Values were reported as cumulative release amount (μg) or percent (%) as a function of time. Metrics associated with burst release and zero order release were determined. All values reported represent average and standard deviation of n=4 samples.

### Stereo Microscopy Imaging

DLS IVRs fabricated were imaged with a Zeiss Stemi 508 Stereo Microscope Labscope. Images were captured and dimensional analysis conducted with Zeiss Labscope software. Strut thickness and band thickness measurements were conducted using ImageJ. Measurements were n=4 for each ring for n=4 samples resulting in 16 total measurements per design. Average and standard deviations were reported.

### Example 2

### Fabrication and Characterization of Diffusion Blocks

Intravaginal rings (IVRs) can be fabricated with geometric complexity to leverage the design freedom associated with additive manufacturing [33]. However, these systems would be too complex to systematically investigate the underlying fundamental aspects of the post-loading process. Therefore, a simplified system defined as ‘diffusion blocks’ was utilized. An overview of the design and fabrication of the simplified system can be seen in FIG. 1. The design of the diffusion blocks can be seen in FIG. 1A where the distances in the Y and Z dimensions are held constant at 20 and 10 mm, respectively, and the distance in the X dimension is varied from 0.5 to 7.6 mm. This was done to assess the effect of diffusion distance on both drug uptake and release. The values were chosen to mimic both the dimensions previously reported in the geometrically complex IVRs as well as a mimic of the cross-sectional diameter of the commercially available IVRs (NuvaRing, 4.0 mm, macaque ring, 6.0 mm, and Estring, 7.6 mm). The dimensions of the blocks are significantly smaller relative to the overall size of the build platform and therefore, many replicates of various dimensions can be fabricated in a single print, as shown. This process was conducted in the user interface of the printer software, which then slices the three dimensional objects into two-dimensional representations that are then fed iteratively to the printer.

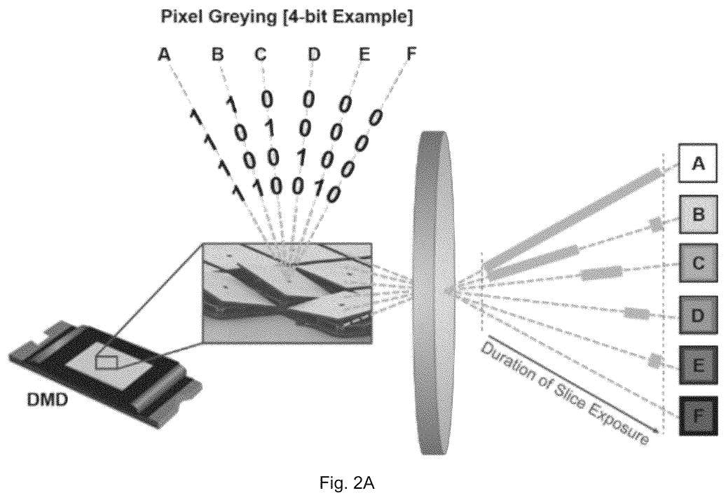

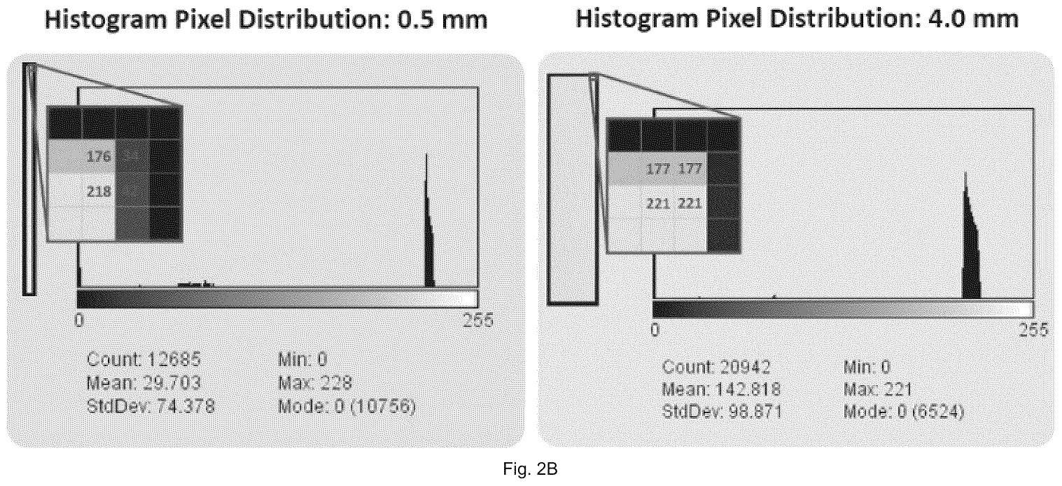

The blocks were fabricated using Digital Light Synthesis (DLS) or as previously published, Continuous Liquid Interface Production (CLIP). This stereolithography system utilizes the interplay of ultra-violet (UV) light and oxygen to selectively polymerize and solidify a photo-active resin. A schematic of this process is shown in FIG. 1B where the digital light projecting (DLP) chip controls the UV projection (λ=385 nm). This is done through a unit at the heart of the DLP called the Digital Micro-Mirror Device (DMD). The DMD takes the input of the two-dimensional slices, overlays the objects onto the micro-mirror grid, and assigns states to each micro-mirror, specifically if the mirror is on (projecting light toward the resin reservoir) or off (projecting light away from the resin). A schematic of this process can be seen in FIG. 2A. As shown in FIG. 1B, the blocks are fabricated with the varied dimension X controlled by the projection from the DMD, with thicker blocks receiving more incident UV light than thinner blocks. The incident light first passes through an oxygen permeable window before proceeding to the photo-active resin. The resin contains a photo-initiator that generates free radicals upon excitation from the incident UV light, which initiates the polymerization reaction. The incorporation of oxygen inhibits the reactivity of the resin by consuming the generated free radicals prior to polymerization, forming a region of uncured resin known as the dead zone (DZ). However, once the concentration of oxygen is sufficiently reduced, the polymerization step becomes favorable and solidification can occur, as shown. As the part is formed, it is pulled from the resin reservoir and the exposure process is repeated.

The photo-active SIL 30 resin used in these studies is a silicone-based resin. The resin consists of two parts, a UV-active component and a thermal-active component, described in FIG. 1C. During the DLS process, the UV active component initiates the polymerization of the (meth) acrylate functional groups within the resin, generating a ‘green’ solid. Following fabrication and residual resin removal, the part is treated to a thermal cure that activates the secondary component to yield the final, functional part.

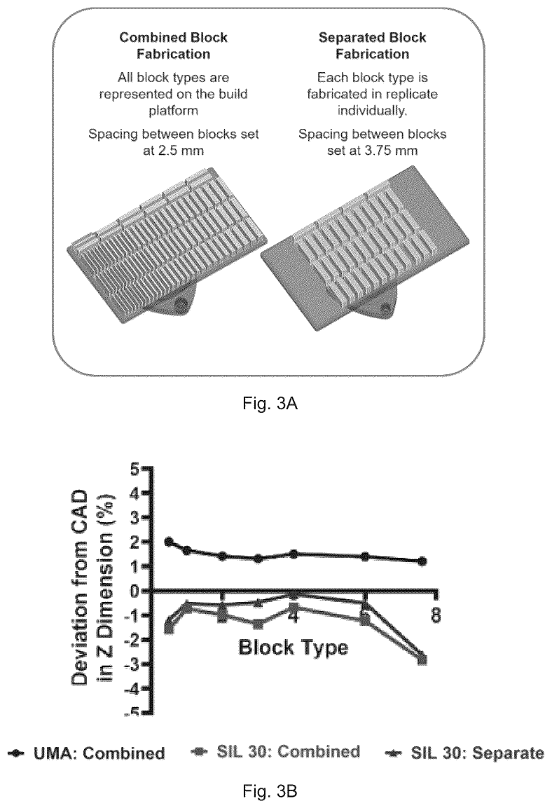

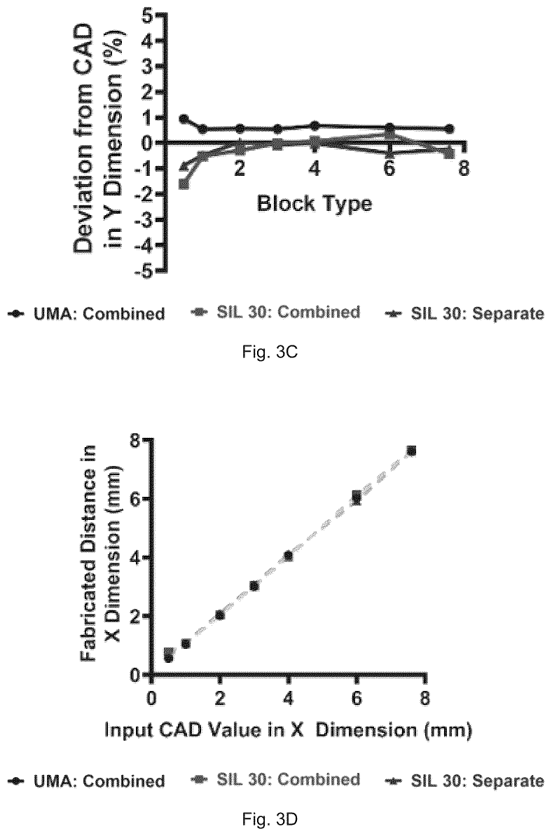

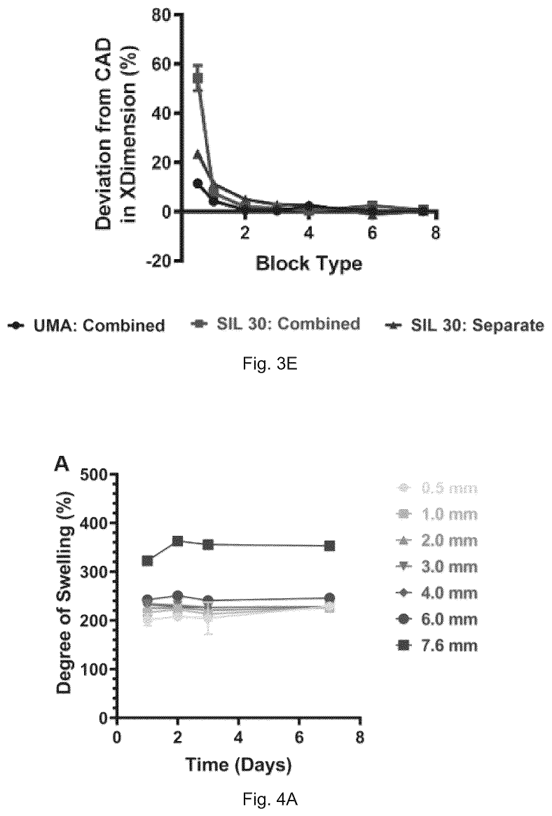

The fabrication of SIL 30 resin with the DLS system has been assessed for geometric fidelity and minimum thresholds established. This includes a minimum wall thickness of 1.5 mm that can be fabricated within acceptable tolerances. The projected pixel, or the area of light illuminated by a single micro-mirror and dictated by the distance between the DMD and the window, of the Ml system used is 75×75 μm. It is possible then to fabricate features below the 1.5 mm threshold however the dimensions of the part may not be as accurate or within acceptable tolerances. To assess the fidelity of the blocks produced using DLS, particularly those falling below the 1.5 threshold, blocks were fabricated both as a function of resin (prototyping and SIL 30) and as a function of placement (combined and separated). The blocks were fabricated in a fast-reacting urethane methacrylate prototyping resin (UMA) to assess the underlining effects of the interplay between the DMD and the fabricated dimension. Because the SIL 30 wall thickness threshold and the interplay between the projected pixel and the fabricated part, two placement orientations were investigated: combined and separated (shown in FIG. 3A). Combined orientation consisted of all block types placed on the build platform for a single print with 2.5 mm spacing (selected such that all blocks fit). Separated orientation consisted of a single block type with controlled spacing of 3.75 mm, an integer value of the projected pixel. High part fidelity to the input CAD was expected in the Y (20 mm) and Z (10 mm) dimensions because the incident light is along the XY-plane and the dimensions are well above any minimum wall thickness thresholds. Part fidelity in these dimensions was assessed as percent deviation from the input value in CAD, shown in FIG. 3B and 3C for the Y and Z dimensions, respectively. For blocks fabricated in UMA, a consistent and slight positive deviation of 2% was observed in both dimensions, regardless of block type. For blocks fabricated in SIL 30 a slight negative deviation was observed in the Y dimension, particularly with the 7.6 mm block, which may be attributed to shrinkage. This was not observed in the Z dimension. For all blocks fabricated in SIL 30, the combined versus separated placement did not have an effect of part fidelity in the Y or Z dimensions, which overall deviated ±3% from the original CAD input.

Blocks were assessed for part fidelity in the variable X dimension both as a function of resin and placement. Analysis of absolute distance in the X dimension is shown in FIG. 3D and was found to be linear for all fabrication conditions. Analysis of percent deviation from the CAD input, shown in FIG. 3E, yielded an inverse relationship between part deviation and block thickness for all fabrication conditions. A baseline over-printing of the part was observed in the UMA resin for the smaller distances, particularly the 0.5 mm blocks. The same placement translated into SIL 30 yielded a significant increase is over-printed, observed for all blocks at or below the 1.5 mm threshold. This over-printing in SIL 30 was found to be reduced once spacing was controlled for in the separated placement condition. The proceeding studies utilized blocks fabricated under separated placement to obtain the highest fidelity parts.

### Example 3

### Characterization of Placebo Blocks During Post-Loading Mimic

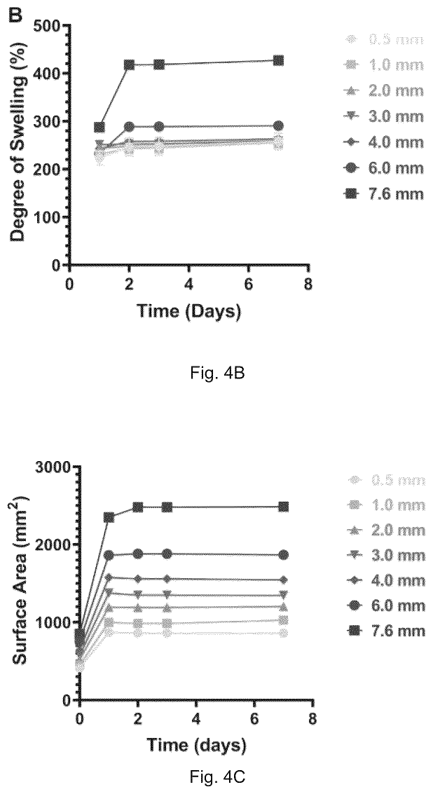

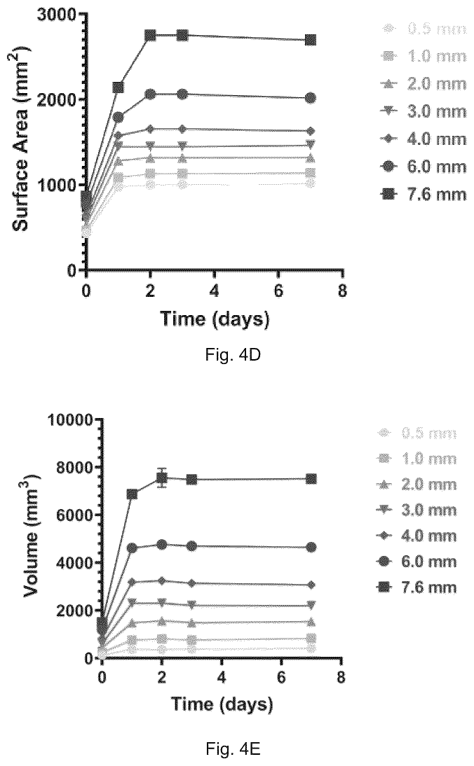

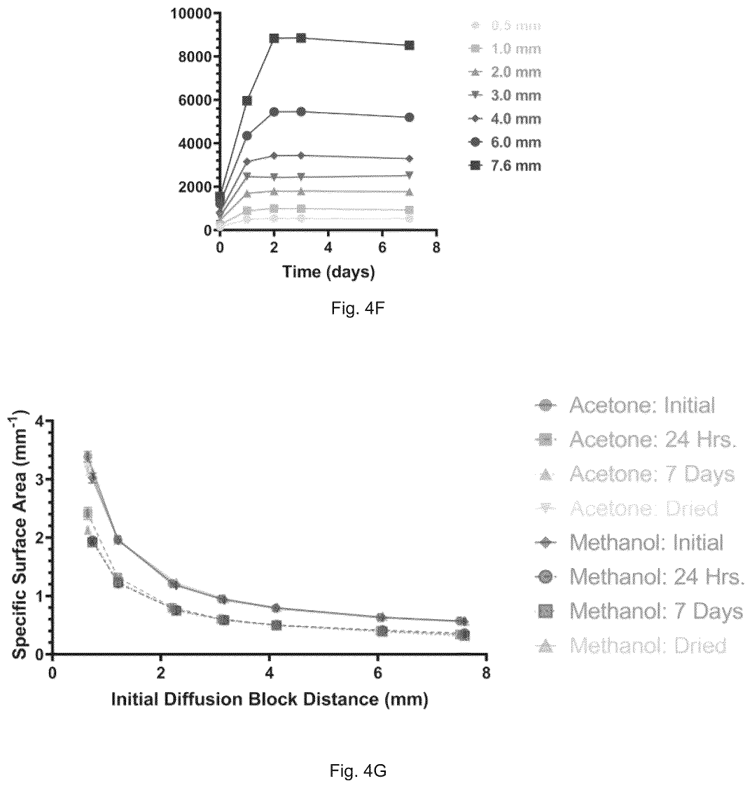

The proposed post-fabrication absorption process involves the exposure of the parts to solvent to swell the SIL 30 matrix and allow for drug intercalation. There are several experimental factors to determine including choice of solvent and exposure duration. Solvents were preliminarily screened and selected based on solvent class, logP, and boiling point, resulting in the selection of methanol (MeOH, Class II) and acetone (Ace, Class III). Exposure duration was determined by immersing all block types in either Ace or MeOH and assessing part metrics (mass and azimuthal distances) on Day 1, 2, 3, and 7. By tracking the degree of swelling as a function of time (FIG. 4A-B), it was found that a plateau of swelling was achieved for all blocks by day 7, indicating that the matrix was fully equilibrated with the surrounding solvent. For block types 0.5-6.0 mm, the maximum swelling was achieved within the first 24 hrs. in both Ace and MeOH. Additionally, the degree of swelling for these blocks was found to be similar at approximately 225±15% for Ace and 237±11% for MeOH. The largest block distance, 7.6 mm, while demonstrating a higher degree of swelling in 24 hrs. than the other blocks in the set, plateaued after 2 days (48 hrs.) at 362±3% for Ace and 418±4% for MeOH.

The collection of azimuthal dimensions enabled the calculations of surface area (SA, mm²) and volume (V, mm³) as a function of swelling time. This is shown for Ace, FIGS. 4C and E, and MeOH, FIG. 4D and F. A similar trend is observed in dimensional metrics as in the degree of swelling where a maximum plateau is reached for most of the block distances within 24 hrs. Unlike degree of swelling, each block type resulted in a unique swelling curve, which is to be expected given the intentional dimensional differences in the X-axis. The block SA and V can be ratioed, resulting in the calculation of specific surface area (SSA, mm⁻¹) for all block types at the initial, swollen (Day 1 and 7) and dried state, shown in FIG. 1G. The smaller block types have a higher relative SA, resulting in a higher SSA compared to the larger blocks. For all block types, as the part swells the volume increases overshadow the surface area increases, resulting in a lower SSA by Day 1, regardless of solvent type. This ratio is maintained during the swelling process, as indicated by the overlap of the Day 7 swollen trace. The parts were then removed from solvent, dried, and final metrics taken, enabling the calculation of a dried SSA. The resulting trace overlaps the initial SSA. These data indicate that all parts swell and maintain a uniform swelling in the presence of solvent in a manner specified by the part's ratio of SA to V. Additionally, once removed, these parts return to their original shape. Collectively, the solvent swelling analysis as a function of time suggests that for most block distances, 24 hrs. is an appropriate solvent exposure duration. Additionally, the cycle of swelling and shrinking the part does not distort the part.

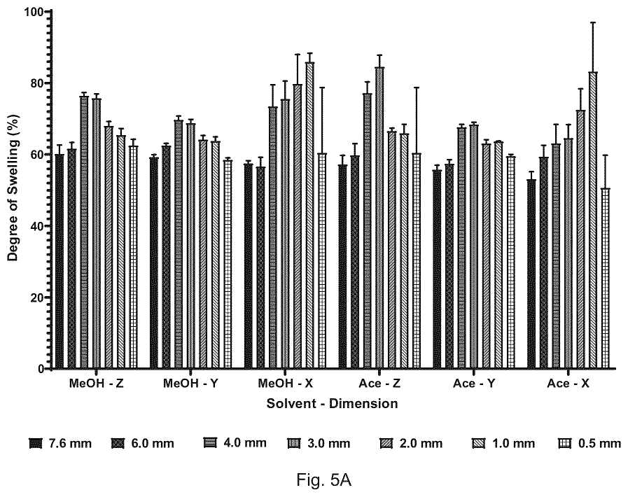

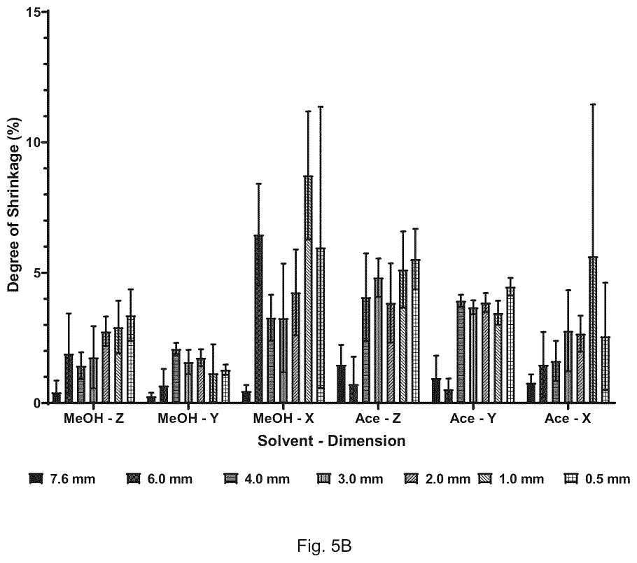

The solvent exposure duration analysis suggested that swelling and drying were uniform based on V and SA tracking however this can be further investigated by tracking the dimensions of each azimuth during the post-loading cycle. This was assessed by immersing all block types in either Ace or MeOH for 24 hr and tracking initial, swollen, and dried metrics in the X, Y and Z dimension. A degree of swelling can be computed for each dimension, shown in FIG. 5A, by block and solvent type. Blocks immersed in MeOH exhibited uniform swelling of 60% in all dimensions for all block types. Blocks immersed in Ace also exhibited uniform swelling in each dimension, but degree of swelling was specific to each block type. The 7.6 mm blocks swelled approximately 70% in each direction with the thinner blocks trending downward to the 0.5 mm blocks at 50% in each direction. For both solvents, uptake and swelling were observed to be uniform along each azimuth. This is likely a result of the combination of DLS fabrication, which has been previously demonstrated to be layerless [28], and the dual curing of the SIL 30 resin itself, resulting in a swellable polymer matrix.

The SSA tracking shown in FIG. 4G suggested that the swollen parts, once dried returned to their original dimensions. This can be assessed more thoroughly by computed percent shrinkage along each azimuth for blocks immersed in either Ace or MeOH. The resulting values are shown in FIG. 5B. Minimal shrinkage (<5%) was observed in the Z and Y dimensions for all block types in both solvents. Slightly higher shrinkage (approaching 10%) was observed in the X dimension for blocks immersed in MeOH. For blocks immersed in Ace, shrinkage was observed to be overall minimal and uniform, however a higher degree of variability was observed in the X dimension (relative to the Z and Y dimensions).

The motivation for the development of post-fabrication drug loading methods was two-fold. First, one goal was to develop an alternative loading route for active pharmaceutical ingredients (APIs) that may not be compatible with the fabrication process due to thermal or UV-light sensitivity. Second, was to streamline precision control over loading particularly as it relates to influencing changes in drug release rate.

At least two aspects of the invention that were assessed included elucidating the relationship between degree of swelling and diffusion distance, and establishing why it exists, and how it influences loading.

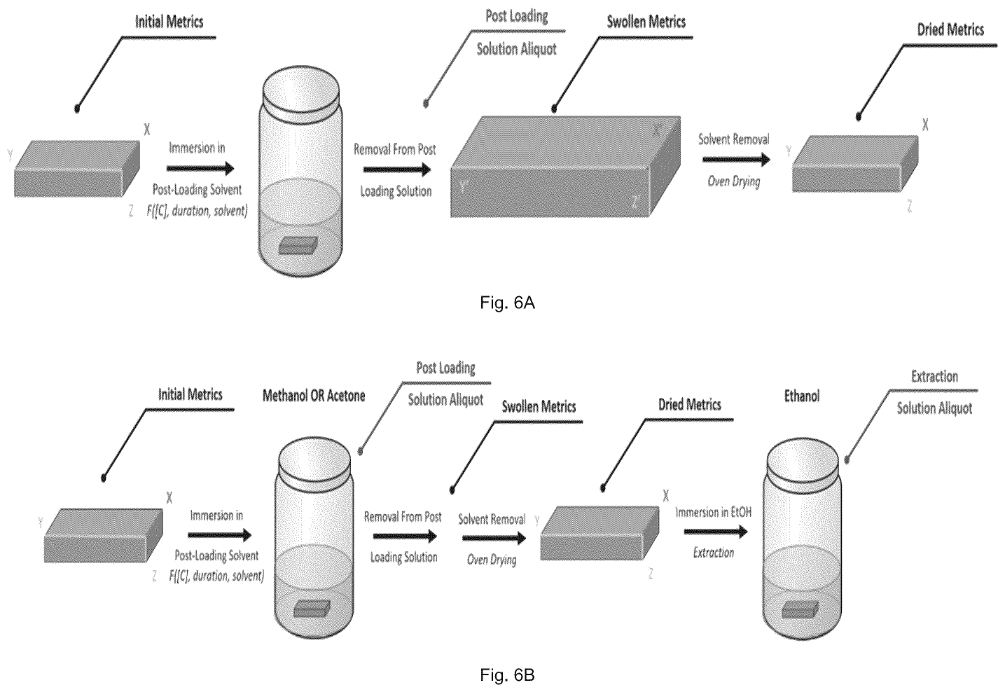

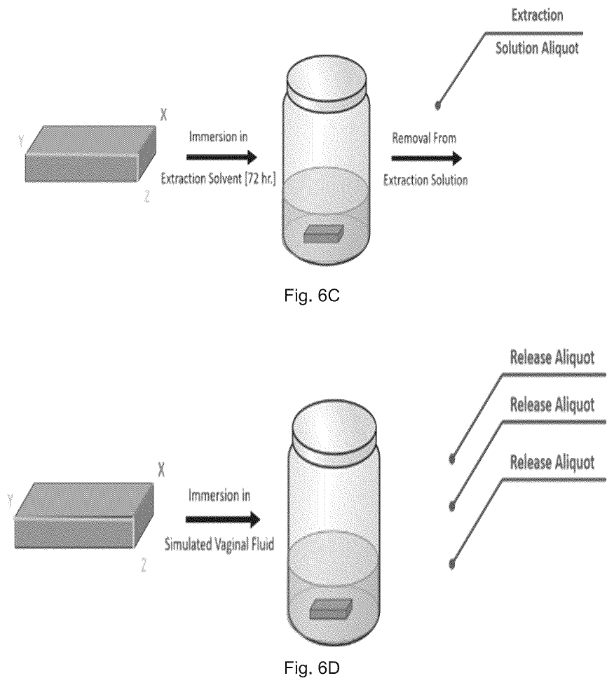

A test part described as a diffusion block with dimensions in X, Y, and Z of 10 mm, 20 mm, and 4 mm, respectively, was primarily used. The general flow of the post-loading process is outlined in FIG. 6A. The initial metrics of dimensional and mass measurements were taken for every block used prior to loading or testing. The block was then immersed in a post-loading solution containing a known concentration of the API for a specified duration. The part was removed and measured for swollen metrics. An aliquot of the post-loading solution following part removal was taken for HPLC analysis. The part was then dried in an oven to remove the post-loading solvent, after which the part was measured for dried metrics. These metrics serve as check points to precisely investigate part behavior throughout the post-loading process, specifically dimensional deformations. Once the part was post-loaded it can either be tested for total drug content via an extraction process, outlined in FIG. 6B, or tested for drug release in simulated vaginal fluid (SVF), outlined in FIG. 6C. Aliquots from both processes are taken for HPLC analysis.

Equation 1 Degree of swelling calculation based on mass. Conventional calculation using the swollen mass (Mₛ) and the dried mass (M_{D}).

$$Degree of Swelling ( \% ) = 100 \times ( {M}_{S} - {M}_{D} / {M}_{D} )$$

Equation 2 Degree of swelling calculation based on dimension. Modified calculation where A represents a measurement along a given axis (X, Y, or Z) in the swollen state (A_{S}) and the dried state (A_{D}).

$$Degree of Swelling ( \% ) = 100 \times ( {A}_{S} - {A}_{D} / {A}_{D} )$$

Following the post-loading procedure with drug, blocks were either treated to EtOH for extraction of β-estradiol or to SVF for release of β-estradiol as a function of time, shown in FIGS. 6C and D, respectively. For these samples, metrics were taken and aliquots were analyzed via HPLC on a C₁₈ column with an optimized method. Extraction aliquots and post-loading solution aliquots were diluted in acetonitrile (ACN) at 1:100 while release samples were analyzed as is. Extracted material includes both the incorporated drug (β-estradiol) as well as leachables/extractables. It was found that β-estradiol was best quantified at 280 nm and additional material, such as resin components, at 265 nm.

There are several potential advantages to post-fabrication loading. First, the swelling of the 3D printed parts in an organic solvent can serve as a removal route of unincorporated extractables and leachables as a step in the device preparation process. Second, it was hypothesized that there exists a region of linearity in which post-loading solvent API concentration correlates with total incorporated amount within the device. This region would enable precision and bespoke loading, adding a second degree of customization to the device. Finally, several drugs can be simultaneously solubilized within a single loading solution, enabling co-formulation of drugs and the preparation of a multipurpose device.

### Example 3

### Tuning and Optimization

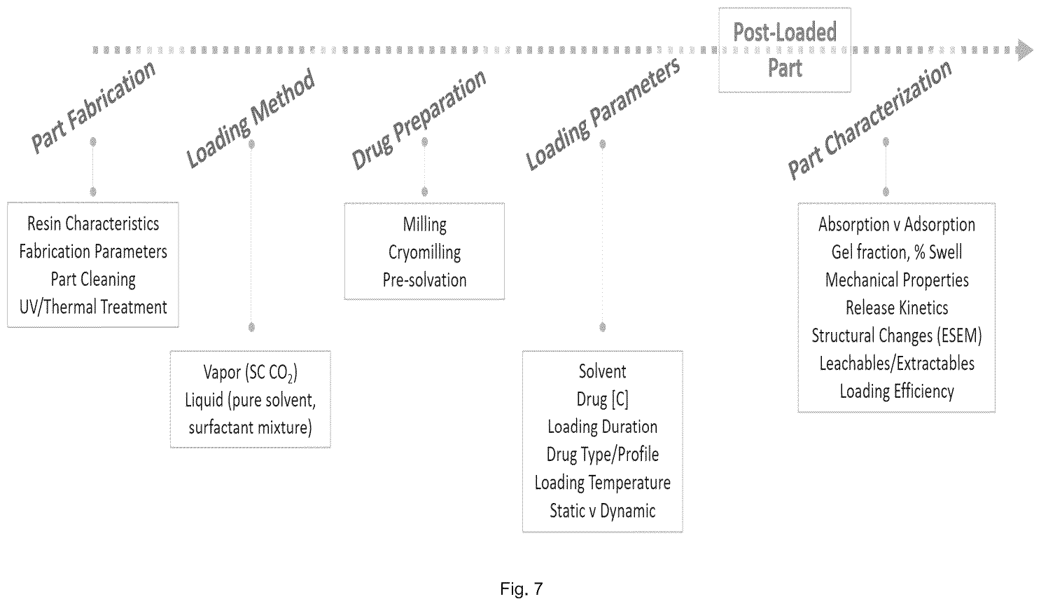

A more detailed breakdown of the potential steps to achieve targeted post-loading amounts and release profiles is shown in FIG. 7. While the part fabrication process was technically outside of the post-loading procedure, variables such as resin type and post-fabrication treatment can dictate factors down the line such as solvent type and post-loading efficiency and was therefore included in the process flow. The process flow depicted in FIG. 7 illustrates the many potential variables that must be considered during the designing of the post-loading process. Therefore, a certain degree of process optimization was necessary in order to correctly identify parameters.

The process flow depicted in FIG. 7 illustrates the many potential variables that must be considered during the designing of the post-loading process. Therefore, a certain degree of process optimization was necessary in order to correctly identify parameters. This example and those following discuss how a few of the process steps were optimized, shedding light on the motivation for the selected parameters in succeeding studies.

### Example 4

### Solvent Selection

The effectiveness of liquid post-loading method (as opposed to supercritical fluids) lies with its ability to penetrate and swell the polymer network of the fabricated part, an aspect largely dictated by solvent-polymer interactions. The resin utilized in IVR fabrication was SIL 30, a highly hydrophobic resin. Therefore, it was likely that solvents with large positive logP values would penetrate and expand the polymer network, quantified via degree of swelling. To investigate this, an exploratory panel of solvents was selected based on FDA solvent classification system (with class III defined as least toxic in residual amounts), logP, and boiling point (° C.), described in Table 1.

The polymerization process is known to result in structures that contain components covalently bonded to the matrix and components trapped during the solidification process, or the soluble fraction. The post-loading process swells the matrix in a manner that allows for the soluble fraction to be removed. In the presence of a drug-solvent solution, the part swelling would represent a two-way street in which the soluble fraction was leached out and the drug was absorbed in. Therefore, it was necessary to understand what fraction remained following post-loading mimic exposure (24 hr.) and full soluble fraction removal (7-day exposure). The calculation of the ratio of the dried mass to the initial mass to determine the matrix fraction (24 hr. exposure) and the gel fraction (7-day exposure) as a function of block type and solvent are shown in Table 1. Both methanol and acetone were observed to have a similar capacity for soluble fraction removal with resulting gel fractions near 0.90. For all blocks except the 7.6 mm type, the 24 hr. exposure appears to be sufficient in removing the bulk of the soluble fraction as evidenced by the similarities between the matrix and gel fractions. This correlates with what was observed during the solvent swelling analysis as a function of time, where swelling equilibration was achieved within the first 24 hrs. The 7.6 mm block types, requiring an additional day to achieve equilibration, exhibited the largest difference between matrix fraction (0.90±0.03) and gel fraction (0.83±0.01), particularly in the case of methanol, which was shown to swell parts upwards of 400%. Collectively these data indicate that 24 hrs. is a sufficient exposure duration for most distances to remove a significant portion of the unincorporated material within the device, which would be left unremoved, would serve as a potential leachable and extractable source.

TABLE 1
Residual resin removal for all block types by solvent in 24 hrs. and 7 days. All block
types (n = 3 per condition) were immersed in MeOh or Ace for either 24 hrs. or
7 days, removed and dried. Fractional amounts were calculated using equations cited
in Section 2.5. Matrix fraction is defined as the amount of soluble, unincorporated
material remaining in the matrix following solvent exposure whereas gel fraction is
defined as the remaining matrix following the complete removal of the soluble fraction.
All values represent average and standard deviations of n = 3 samples per condition.

| | Solvent: Methanol | | Solvent: Acetone | |
|---|---|---|---|---|
| Block Type by X | Matrix Fraction | Gel Fraction | Matrix Fraction | Gel Fraction |
| Dimension (mm) | (24 hr Exposure) | (7 Day Exposure) | (24 hr Exposure) | (7 Day Exposure) |
| 7.64 ± 0.03 | 0.90 ± 0.03 | 0.83 ± 0.01 | 0.92 ± 0.03 | 0.87 ± 0.01 |
| 6.02 ± 0.10 | 0.91 ± 0.01 | 0.89 ± 0.01 | 0.91 ± 0.01 | 0.92 ± 0.01 |
| 4.01 ± 0.38 | 0.93 ± 0.01 | 0.91 ± 0.01 | 0.94 ± 0.01 | 0.93 ± 0.01 |
| 3.00 ± 0.38 | 0.93 ± 0.01 | 0.91 ± 0.01 | 0.94 ± 0.01 | 0.93 ± 0.01 |
| 1.99 ± 0.33 | 0.94 ± 0.01 | 0.92 ± 0.01 | 0.94 ± 0.01 | 0.93 ± 0.01 |
| 1.17 ± 0.16 | 0.94 ± 0.02 | 0.91 ± 0.02 | 0.94 ± 0.01 | 0.93 ± 0.01 |
| 0.75 ± 0.05 | 0.93 ± 0.03 | 0.90 ± 0.05 | 0.94 ± 0.01 | 0.93 ± 0.02 |

The exploratory solvent study with whole rings indicates that there was a tradeoff between degree of swelling and imparted defects on the ring that must be considered when moving forward. It should be noted that this panel was specific to the SIL 30 resin utilized in fabrication. The solvents carried forward for the pilot post-loading study were methanol, acetone, isopropyl acetate and chloroform. Acetone and methanol both yielded rings of comparable gel fractions, comparable swelling, and did not severely damage the ring in the process. Isopropyl acetate is a known class III solvent and damaged the ring after 4 days of immersion, a duration exceeding the targeted loading time. Chloroform was observed to be the most aggressive solvent in terms of degree of swelling and therefore will be carried forward as a ‘best case scenario’ solvent. The solvents selected from this panel have varying degrees of swelling, log P as well as drug solubility and therefore, performance in terms of total loading and loading efficiency can only be partly attributed to solvent uptake.

### Example 4

### Post-Loading Soak Duration

The exploratory solvent panel yielded four solvents of potential interest to investigate further in terms of soak duration of a solvent loaded with a model drug, β-Estradiol. To further simplify the system and enable quantitative check points (as opposed to previous qualitative observations), whole IVRs were replaced with unloaded diffusion blocks, with dimensions outlined in Table 2. Blocks utilized in soak duration study were fabricated from a single batch file and the initial metrics of dimensions in each direction and mass were taken. The average and standard deviations as well as the percent relative standard deviations (% RSD) are shown for each metric in Table 2. Collectively, the initial metrics suggest there is minimal variability in the sample set prior to solvent treatments.

TABLE 2
Combined dimensional and mass metric analysis for all test blocks utilized in pilot post-loading study.
The dimensions are described in the schematic with the direction of the print denoted. The average
and standard deviations for specified metrics were calculated and represent n = 16 samples.

| | X (mm) | | Y (mm) | | Z (mm) | | Mass (mg) | |
|---|---|---|---|---|---|---|---|---|
| | Avg ± Std | % RSD | Avg ± Std | % RSD | Avg ± Std | % RSD | Avg ± Std | % RSD |
| All | 9.94 ± 0.05 | 0.55 | 19.94 ± 0.08 | 0.40 | 4.01 ± 0.03 | 0.78 | 883.2 ± 9.6 | 1.1 |
| Samples | | | | | | | | |

Diffusion blocks were incubated in respective solvents super saturated with β-Estradiol (20 mL per block). Soak durations were set at 8, 24 and 48 hr. A control block was soaked in a drug-free solvent for 48 hr. A sectioned NuvaRing (EVA) segment of comparable mass was concurrently tested to serve as a known benchmark. Blocks were immersed for indicated durations and swollen metrics taken. Blocks were dried, metrics assessed and placed in EtOH for three days to facilitate extraction of the loaded β-Estradiol. Ethanol was selected as an extraction solvent given previous observation of degree of swelling as well as high solubility of β-Estradiol.

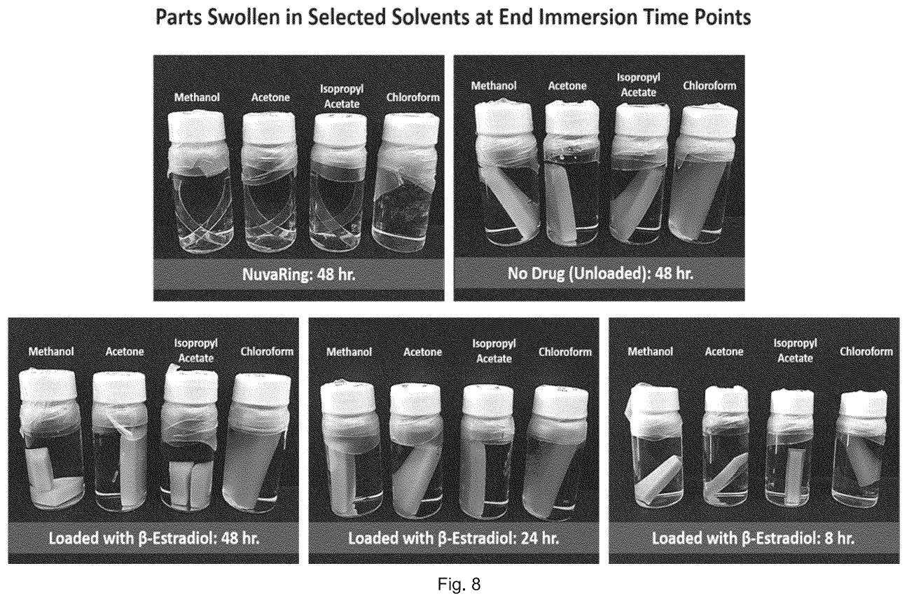

Parts (diffusion block and NuvaRing sections) were incubated in respective solvents for the prescribed time. Images of the swollen parts at the conclusion of the immersion are shown in FIG. 8. Overall, the swelling in different solvents did not appear to distort the diffusion blocks with the exclusion of the 48 hr loaded condition in which methanol and chloroform contorted the blocks such that they broke in half. The differences in the degrees of swelling as a function of soak duration is visible between the 48 and 8 hr time points. Finally, the chloroform completely disintegrated the NuvaRing section, preventing further analysis.

Diffusion blocks were quantitatively assessed throughout the post-loading process by documenting dimensions via calipers and mass. The dried masses of the blocks were evaluated relative to the initial masses as a function of solvent type and sample condition, shown in Table 3. This is a similar assessment as gel fraction however does not completely describe the samples when the solvents are loaded with β-estradiol. The true gel fraction of the parts at 48 hr is highlighted in green with the unloaded sample condition. These values are similar and slightly elevated relative to the values obtained previously with rings following a 6-day incubation, as to be expected. Values above 1 indicate mass was gained following the post-loading processes. It should be noted that all solvents are capable of removing the soluble fraction (to varying degrees) and therefore mass can be acquired via drug uptake without achieving a fraction above 1. The NuvaRing sections that were recovered had a gel fraction of 1.

TABLE 3
Analysis of the ‘gel fraction' comparing the dried mass of the parts to the initial
mass as a function of solvent type. Because the solutions were loaded, the only true gel
fraction of the diffusion blocks was observed with the unloaded control, highlighted in green.

| Sample Condition | Methanol | Acetone | Isopropyl Acetate | Chloroform |
|---|---|---|---|---|
| NuvaRing Section: | 1.00 | 1.00 | — | — |
| 48 hr. | | | | |
| Unloaded | 0.90 | 0.89 | 0.91 | 0.90 |
| Solvent: 48 hr. | | | | |
| β-Estradiol: 48 hr. | 0.97 | 1.00 | 0.99 | 0.90 |
| β-Estradiol: 24 hr. | 0.98 | 1.02 | 1.00 | 0.95 |
| β-Estradiol: 8 hr. | 1.01 | 1.04 | 1.02 | 0.95 |

The unloaded 48 hr sample was compared to the loaded 48 hr sample for all solvent types at all stages of the post-loading process. The masses are shown in Table 4 and compared. Percent increase was calculated of the loaded sample relative to the unloaded. Samples were initially similar in masses prior to treatment. Additionally, samples exhibited similar swelling behavior for all solvents, with the potential exception of isopropyl acetate. Methanol, acetone, and isopropyl acetate yielded samples with an increased mass of the dried loaded part relative to the unloaded control. Chloroform yielded a comparative mass loss.

TABLE 4
Combined table comparing the unloaded 48 hr control to the loaded
48 hr sample as a function of solvent type at different stages of
the post-loading process. The percent increase is of the loaded sample
relative to the unloaded sample. This analysis was a pilot study
and therefore represents n = 1 measurement for each condition.

| | | |
|---|---|---|
| Unloaded | Loaded | Increase (%) |

Methanol

| | | | |
|---|---|---|---|
| Initial Mass (mg) | 867.7 | 888.2 | 2.36 |
| Swollen Mass (mg) | 3228.8 | 3222.1 | −0.21 |
| Dry (mg) | 779.5 | 859.6 | 10.28 |

Acetone

| | | | |
|---|---|---|---|
| Initial Mass (mg) | 868.7 | 887.3 | 2.14 |
| Swollen Mass (mg) | 2974.1 | 3239.1 | 8.91 |
| Dry (mg) | 775.0 | 891.5 | 15.03 |

Isopropyl Acetate

| | | | |
|---|---|---|---|
| Initial Mass (mg) | 870.4 | 872.0 | 0.18 |
| Swollen Mass (mg) | 3639.3 | 3752.5 | 3.11 |
| Dry (mg) | 793.1 | 859.1 | 8.32 |

Chloroform

| | | | |
|---|---|---|---|
| Initial Mass (mg) | 892.2 | 884.3 | −0.89 |
| Swollen Mass (mg) | 9961.3 | 9908.6 | −0.53 |
| Dry (mg) | 799.7 | 796.8 | −0.36 |

The solutions used for post-loading were evaluated via HPLC to determine concentration of β-Estradiol. The values averaged were samples collected following the post-loading process which were then compared to the original (unused) post-loading solutions. The total loading concentrations vary depending on the solubility of β-Estradiol within each solvent therefore this analysis is best served by calculating the % RSD within each sample set, shown in Table 5. These values are all acceptably low expect within the case of chloroform. It should be noted where other solvents were clear after β-Estradiol incorporation, chloroform was cloudy. This could be a factor in the variability of the loading concentration.

TABLE 5
HPLC analysis of post-loading solutions. Solutions as a function
of post-loading solvent concentration values are compiled as
a function of sample condition following the loading procedure.
Values are compared to initial post-loading solution values.

| Post-Loading | | [β-Estradiol] | |
|---|---|---|---|
| Solvent | Sample Condition | μg/mL | % RSD |
| Methanol | Original Solution | 5467 | |
| | Post-Loading Solution | 5121 ± 178 | 3.48 |
| Acetone | Original Solution | 5691 | |
| | Post-Loading Solution | 5512 ± 120 | 2.18 |
| Isopropyl | Original Solution | 4651 | |
| Acetate | Post-Loading Solution | 4404 ± 116 | 2.64 |
| Chloroform | Original Solution | 2365 | |
| | Post-Loading Solution | 1679 ± 308 | 18.37 |

### Example 6

### Quantification of Metrics Throughout Post-Loading Process

To demonstrate reproducibility of the process, diffusion blocks were treated to the optimized post-loading procedure using methanol, acetone, and isopropyl acetate. The initial metrics obtained for each solvent condition are compiled in Table 6. It can be seen that as with the pilot study, little sample variation exists between the blocks, represented by the low % RSD values.

TABLE 6
Initial metrics obtained in reproducibility study as
a function of solvent condition. Each solvent represents
average and standard deviation of n = 7 samples
with the percent relative standard deviation shown below.

| | X (mm) | Y (mm) | Z (mm) | Mass (mg) |
|---|---|---|---|---|
| Methanol | 9.92 ± 0.13 | 19.97 ± 0.08 | 4.13 ± 0.05 | 882.3 ± 11.6 |
| | (1.34%) | (0.42%) | (1.21%) | (1.3%) |
| Acetone | 9.95 ± 0.10 | 20.02 ± 0.05 | 4.09 ± 0.03 | 880.0 ± 14.3 |
| | (0.96%) | (0.26%) | (0.68%) | (1.6%) |
| Isopropyl | 9.96 ± 0.06 | 20.00 ± 0.09 | 4.11 ± 0.04 | 884.5 ± 17.8 |
| Acetate | (0.57%) | (0.44%) | (0.91%) | (2.0%) |

The post-loading process was conducted utilizing the 24 hr time point and the swollen metrics of the blocks taken following removal from solvent treatment. The obtained values are shown in Table 7. Given the different logP values associated with each solvent and therefore the different propensities to swell the SIL 30 matrix, similar values between solvent conditions was not expected. Rather, to assess the reproducibility of the solvent, the variation observed within each sample condition was determined to be of interest. As shown in Table 7, there exists little sample variation within each solvent condition as denoted by the low % RSD values. While these values are slightly elevated relative to the initial, they are still acceptably low. This suggests that the blocks are behaving similarly during the solvent swelling and drug uptake step of the process and can be confirmed through quantification of total extracted drug.

TABLE 7
Swollen metrics obtained in reproducibility study as
a function of solvent condition. Each solvent represents
average and standard deviation of n = 7 samples
with the percent relative standard deviation shown below.

| | X (mm) | Y (mm) | Z (mm) | Mass (mg) |
|---|---|---|---|---|
| Methanol | 16.09 ± 0.17 | 32.28 ± 0.42 | 6.68 ± 0.17 | 3106.9 ± 98.8 |
| | (1.04%) | (1.31%) | (2.58%) | (3.2%) |
| Acetone | 16.16 ± 0.19 | 32.36 ± 0.36 | 6.71 ± 0.18 | 3121.7 ± 72.6 |
| | (1.18%) | (1.10%) | (2.69%) | (2.3%) |
| Isopropyl | 16.45 ± 0.45 | 33.11 ± 0.77 | 7.18 ± 0.49 | 3487.8 ± 308.5 |
| Acetate | (2.72%) | (2.33%) | (6.85%) | (8.8%) |

The slightly more controlled method of drying was utilized following removal from loading solvent, enabling the quantification of metrics after the drying step was completed due to lack of distortions observed in the samples. The obtained values are compiled in Table 8. As with the previous checkpoints, little sample variation was observed within each solvent condition. Furthermore, the blocks appeared to have returned to remarkably similar values in terms of dimension and mass, regardless of sample treatment.

TABLE 8
Dried metrics obtained in reproducibility study as a
function of solvent condition. Each solvent represents
average and standard deviation of n = 7 samples
with the percent relative standard deviation shown below.

| | X (mm) | Y (mm) | Z (mm) | Mass (mg) |
|---|---|---|---|---|
| Methanol | 9.86 ± 0.15 | 19.98 ± 0.20 | 4.11 ± 0.03 | 875.4 ± 11.9 |
| | (1.52%) | (1.02%) | (0.78%) | (1.4%) |
| Acetone | 9.96 ± 0.08 | 20.09 ± 0.12 | 4.14 ± 0.04 | 898.9 ± 17.2 |
| | (0.83%) | (0.62%) | (0.91%) | (1.9%) |
| Isopropyl | 9.94 ± 0.08 | 19.97 ± 0.16 | 4.13 ± 0.11 | 882.8 ± 24.3 |
| Acetate | (0.81%) | (0.80%) | (2.66%) | (2.8%) |

Results of total drug loaded, weight percent loading and loading efficiency were quantified and calculated, shown in Table 9. The total drug loaded and weight percent loaded (determined by normalizing the extracted drug to the mass of the dried block) yielded values in line with those observed during the pilot study. The pre-loaded maximum for β-Estradiol was determined to be 10 wt. % and the below values indicate that the post-loading method was roughly on par. Importantly, the loading procedure yielded minimal sample variability, particularly with methanol and acetone, suggesting the developed method to be robust and reproducible. Finally, the loading efficiencies, calculated utilizing the [β-Estradiol] of the loading solution following immersion, indicated similar uptake between solvents. The post-loading solution was super saturated with β-Estradiol; therefore, it is possible that these efficiencies could improve with an optimized concentration of loaded drug.

TABLE 9
Total amount of β-Estradiol extracted from post-loaded diffusion
blocks (n = 3) as a function of loading solvent. Values were
obtained via HPLC analysis of a 10 μL aliquot, extrapolated
to determine total loading. Weight percent was calculated by utilizing
the dried mass of the block. Loading efficiency was calculated
utilizing the [β-Estradiol] determined in the used post-
loading solution for each sample. Values represent average ±
standard deviation (% RSD) for n = 3 samples per solvent condition.

| | β-Estradiol | Wt. % | Loading |
|---|---|---|---|
| | Post Loaded (mg) | by Part Mass (%) | Efficiency (%) |
| Methanol | 42.9 ± 1.0 | 4.9 ± 0.1 | 17.3 ± 0.8 |
| | (2.3%) | (1.8%) | (4.9%) |
| Acetone | 70.4 ± 2.0 | 7.7 ± 0.2 | 19.1 ± 1.3 |
| | (2.8%) | (2.4%) | (6.7%) |
| Isopropyl | 41.0 ± 1.1 | 4.7 ± 0.3 | 20.6 ± 1.2 |
| Acetate | (2.6%) | (7.2%) | (5.7%) |

### Example 7

### Release in Simulated Vaginal Fluid

The release properties of parts both pre-and post-loaded with β-Estradiol were investigated in simulated vaginal fluid (SVF). All parts were between 800-900 mg and therefore the volume of SVF used for all release studies was 60 mL. Aliquots of 1 mL were removed for HPLC analysis and the medium replenished. Analysis was conducted against β-Estradiol standards prepared in ACN and peaks quantified at 280 nm.

### Release Profiles of Pre-Loaded Diffusion Blocks

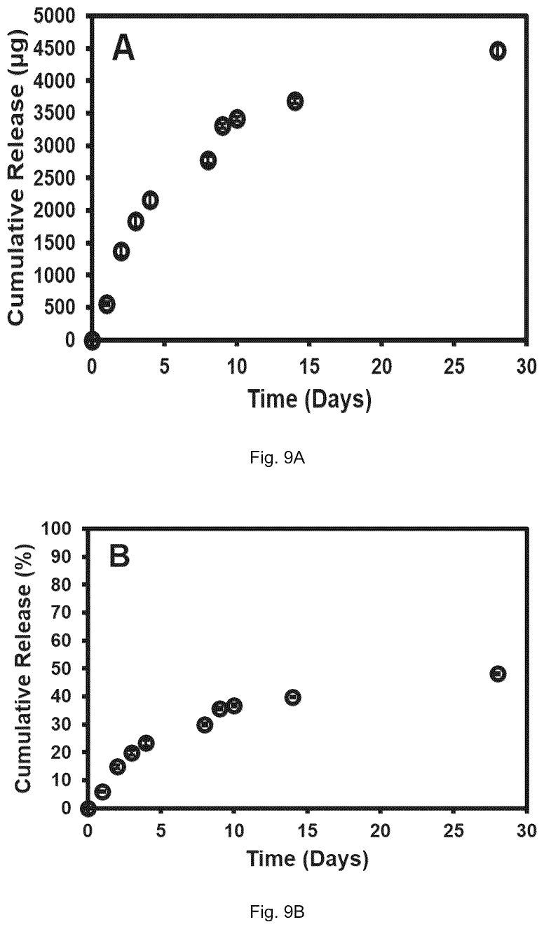

Diffusion blocks fabricated with a pre-loaded SIL 30 resin containing 1 wt. % β-Estradiol. Blocks were placed in SVF and aliquots analyzed via HPLC. The resulting profiles are shown in FIG. 9A-B as cumulative release by μg and by percent, respectively. It should be noted that the blocks are 4 mm thick, a much larger diffusion distance when compared to the wall thicknesses of the IVRs. Therefore, it was not surprising that the blocks released only 48% of the total over the course of 28 days. The relative standard deviations associated with these measurements are all below 5%, suggesting the pre-loaded blocks have minimal release kinetic variability.

### Release Profiles of Post-Loaded Diffusion Blocks

The release kinetics of diffusion blocks post-loaded as a function of solvent type were quantified similar to the pre-loaded blocks. The resulting analyses are shown in FIG. 10A-B as cumulative release by μg and cumulative percent release, respectively. The relative standard deviations associated with these measurements are 6-14% for methanol, 17-28% for acetone, and 4-6% for isopropyl acetate. This degree of variability is elevated relative to their pre-loaded counterparts. For methanol and isopropyl acetate, the same variation decreases with time. Contrastingly, acetone increases in variability over the duration of the release study. The diffusion blocks appear to release similarly within error independent of solvent loading type, however true deviations could become more apparent over the duration of the release study.

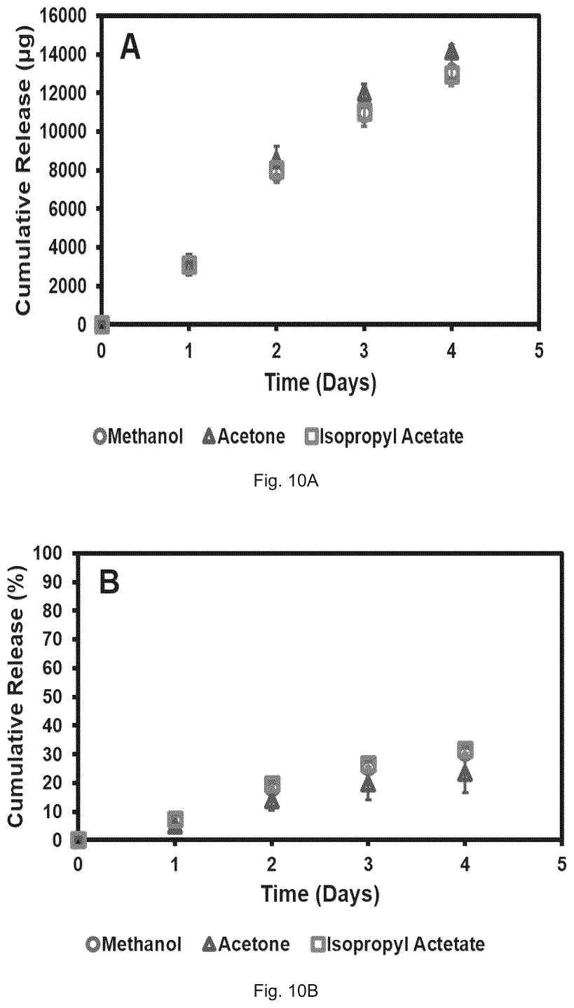

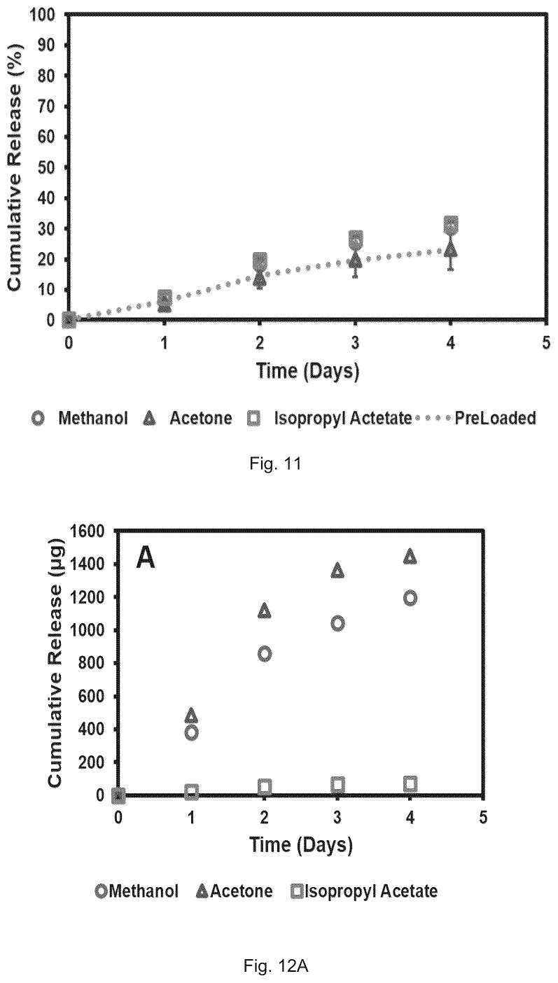

The percent cumulative release of the post-loaded blocks was plotted against the pre-loaded average for the first four days, shown in FIG. 11. The cumulative percent release is similar between the two loading methods. It should be noted that the pre-loaded weight percent is 1 wt. % compared to the 4-7 wt. % of the post-loaded blocks.

### Release Profiles of Post-Loaded Estring Sections

In a similar post-loading study was conducted on Estring sections of comparable mass to the diffusion blocks (n=1). The cumulative release by μg and cumulative percent release is shown in FIG. 12A-B, respectively. Contrasting to the post-loaded blocks, a noticeable difference was observed between the solvent types in terms of cumulative release by μg. This difference is less noticeable by cumulative percent release. Furthermore, the Estring sections appear to rapidly release their payload, relative to CLIP-fabricated parts, shown in FIG. 11. The Estring was chosen as a benchmark given the similar PDMS composition.

### Example 8

### Successive Loading

The ability to successively load a series of blocks utilizing the same loading solution was investigated. The post-loading solvent procedure utilizes 20 mL of solution per block, which depending on the solvent, will uptake between 1-2.5 mL of solvent. Therefore, the 20 mL, while reduced, is not fully used up in a single loading cycle. Given the potential monetary savings, it would be ideal to recycle or reuse the post-loading solvent for successive loading.

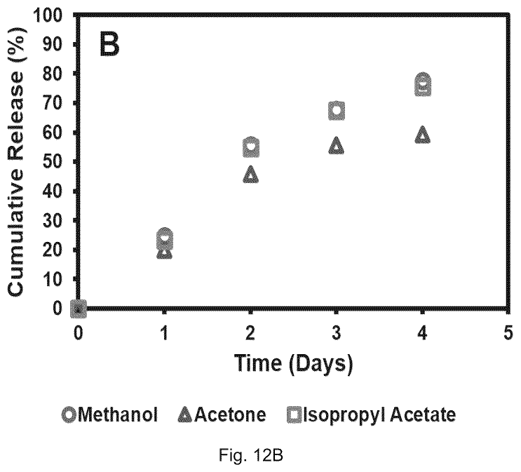

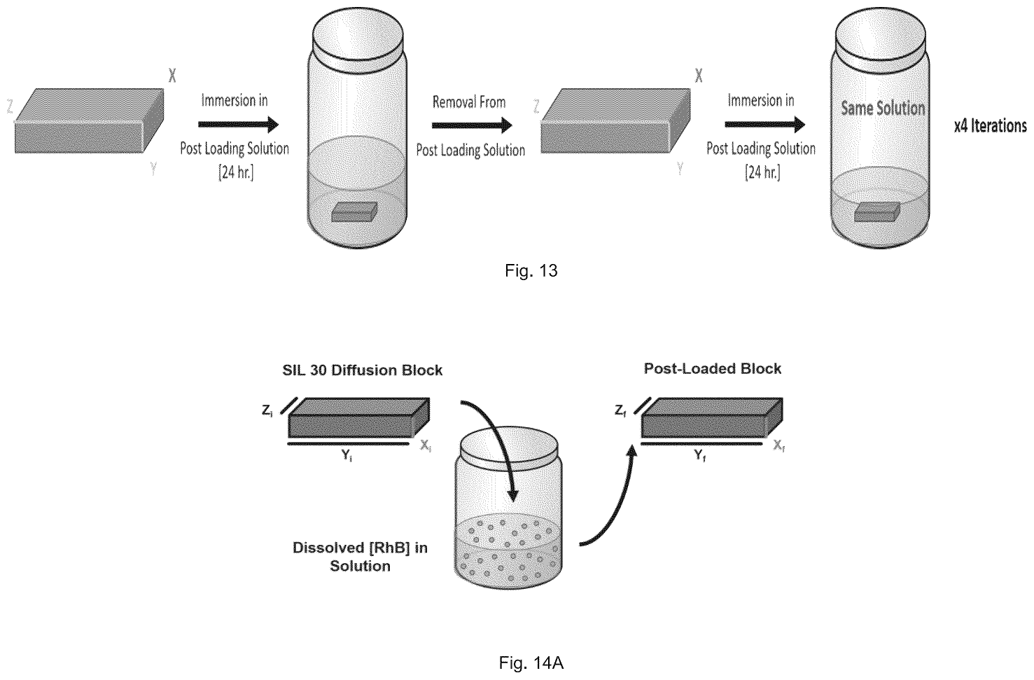

The procedure of successive loading is outlined in FIG. 13. In this procedure, a single jar containing 20 mL of methanol super saturated with β-Estradiol was iteratively loaded every 24 hr. with a fresh diffusion block. This was done for four cycles to yield four blocks, denoted by loading order.

### Metric Quantification During Successive Loading Process

Throughout the post-loading process, blocks were assessed utilizing methods established in Section 2 and 3. To identify differences in loading during the iterative process, blocks were evaluated based on uptake and extraction.

Solvent uptake was calculated following removal from the post-loading solution and values compiled as degree of swelling in Table 10. No true trend in terms of decreased degree of swelling was observed for the dimensions of the blocks as a function of loading order. However, there was a notable decrease in percent mass increase as loading order progresses. It should be noted, however, that the variability is percent mass uptake is fairly small at 3.74%. Overall, there was minimal indication that the behavior of the blocks in the loading solution varies significantly as a function of loading order.

TABLE 10
Degree of swelling for blocks successively loaded in MeOH for 24 hrs. each.
Loading order is denoted by number. The average, standard deviation, and
percent relative standard deviation values represent n = 4 samples.

| Sample Name | X (%) | Y (%) | Z (%) | Mass (%) |
|---|---|---|---|---|
| Loading Round 1 | 53.71 | 54.09 | 42.05 | 190.32 |
| Loading Round 2 | 47.47 | 47.04 | 46.15 | 180.08 |
| Loading Round 3 | 45.84 | 51.89 | 46.73 | 177.60 |
| Loading Round 4 | 48.75 | 46.60 | 42.93 | 174.82 |
| Average ± | 48.94 ± 3.39 | 49.91 ± 3.68 | 44.47 ± 2.32 | 180.71 ± 6.76 |
| Standard Deviation | | | | |
| % RSD | 6.93 | 7.37 | 5.22 | 3.74 |

Blocks were further assessed by quantifying the extracted drug via HPLC. Values obtained are compiled in Table 11. The total amount of drug extracted, weight percent loading and loading efficiency were calculated and averaged, with variation described by % RSD. Overall, a slight decrease in loading metrics was observed as a function of loading order. However, once averaged out, these values yielded minimal variability. Additionally, values shown are in-line with those obtained during the reproducibility study (Section 3.2) for methanol.

TABLE 11
Quantification of β-Estradiol extracted from successively loaded
blocks via HPLC. Block order is denoted numerically. Total amount
extracted was determined from 10 μL aliquot of the extraction
solution. Weight percent loading was calculated using dried block
mass. Loading efficiency was calculated from an aliquot of the
post-loading solution taken after each loading cycle. Average,
standard deviation and % RSD values represent n = 4 samples.

| | Amount | Wt. % | Loading |
|---|---|---|---|
| Sample Name | (mg) | Loading (%) | Efficiency (%) |
| Loading Round 1 | 44.3 | 5.0 | 17.9 |
| Loading Round 2 | 42.3 | 4.9 | 17.1 |
| Loading Round 3 | 42.2 | 4.8 | 17.1 |
| Loading Round 4 | 41.8 | 4.7 | 16.9 |
| Average ± | 44.3 ± 1.1 | 4.8 ± 0.1 | 17.3 ± 0.4 |
| Standard Deviation | | | |
| % RSD | 2.6 | 2.7 | 2.7 |

Within error, minimal differences were observed in drug loading of successively loaded blocks as a function loading order were observed. This study was conducted using a single solution of 20 mL for four cycles. This represents approximately 8.5-10 mL of solvent uptake leaving 10 mL necessary to fully coat the bottom of the sample container. Given the residual volume, more iterations could be possible however is constrained by the sample container. Thus, it could be possible to recycle the post-loading solution for several cycles rather than reloading the same sample container.

The successive loading study iteratively loaded four diffusion blocks with a single loading solution and represents a pilot concept to reuse loading solution. Samples yielded similar uptake and total loading suggesting that the successive loading is feasible. Additionally, the values obtained are in-line with the reproducibility study for methanol. A likely application would be toward the recycling of post-loading solutions given the consistent loading between cycles.

### Example 9

### Visualizing the Loading Process

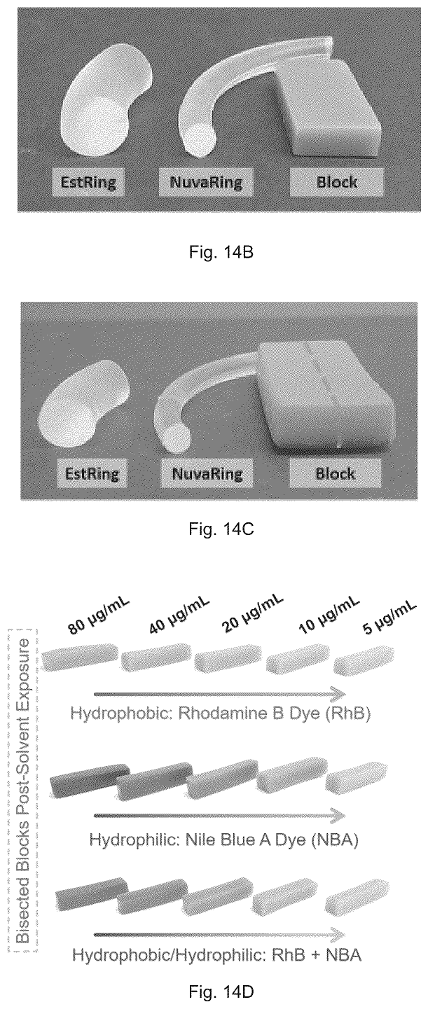

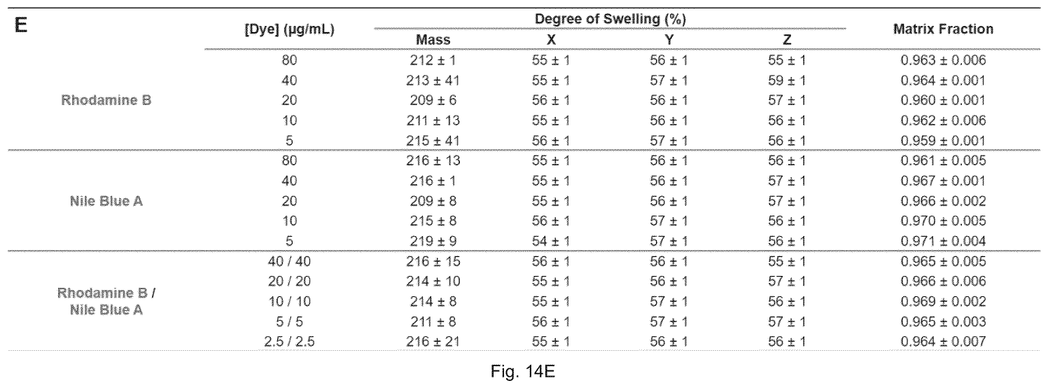

Solvent swelling analysis indicated the uniform swelling and shrinking of the matrix but it was necessary to understand if API uptake would be homogenous therefore blocks were exposed to dyes to allow for visualization of the loading process. Blocks (Xᵢ=4.0 mm) were exposed to a rhodamine B (RhB) methanol solution for 24 hrs as shown in schematic FIG. 14A. For comparison, sections of placebo commercially available injected molded Estring (silicone) and Nuvaring (ethylene-vinyl acetate, EVA) of approximately similar mass (0.8 g) were also exposed to the RhB/MeOH solution. Images of the pre-solvent and post-solvent exposure can be seen in FIGS. 14B and 14C, respectively. For the injected materials, minimal swelling was observed and consequently RhB was not detected following extraction. Conversely, the 4.0 mm block type swelled approximately 200%, resulting in observable uptake. This suggests that DLS SIL 30 materials are unique suited for the post-loading process in methanol. It could be possible to swell injected molded materials in other organic solvents [34], however these solvents may not be Class II/III, and therefore could complicate the transition into clinic.

To further investigate the visualization of the post-loading process, three exposure conditions were generated in methanol: hydrophobic dye (rhodamine B, RhB), hydrophilic dye (nile blue A, NBA) and hydrophobic/hydrophilic combination (RhB/NBA). These solutions, starting at 80 μg/mL were serial diluted down to 5 μg/mL, resulting in 5 solutions per dye condition. It should be noted that the combination solution held total dye amount constant resulting in half of the initial individual dye concentration. Blocks of the 4.0 mm type (n=3 condition) were exposed to these solutions for 24 hrs. and removed. Upon removal, blocks were bisected, as indicated by the dashed line in FIG. 14C, and imaged according to condition, as shown in FIG. 14D. It can be visually observed that dye uptake correlates with decreasing dye concentration. Swelling analysis was conducted in the presence of dye and tabulated in FIG. 14E by loading condition. The degree of swelling by mass and azimuth are independent of both dye type (hydrophobic vs. hydrophilic) and dye concentration. Additionally, as shown in FIG. 14A, the 4.0 mm block types swell uniformly in the X, Y, and Z dimensions. The matrix fractions observed are similar across all block loading conditions at 0.96, a higher value than observed under post-loading mimic conditions in Table 1 (0.93) indicating that there was dye uptake. The authors do not recommend back calculating uptake by subtracting dye-loaded matrix fraction from place matrix fraction. These data demonstrate both the complete intercalation of the dye within the SIL 30 matrix as well as the ability to predictably tune total loading by controlling of the initial post-loading solvent concentration.

Initial tests investigated post-loading parts as a function of material (EstRing, NuvaRing, SIL 30 block) in a Rhodamine B/Methanol solution. The analysis was further expanded to an investigation as a function of RhB concentration in methanol for SIL 30 blocks. Swelling metrics and extracted RhB were quantified as a function of loading concentration.

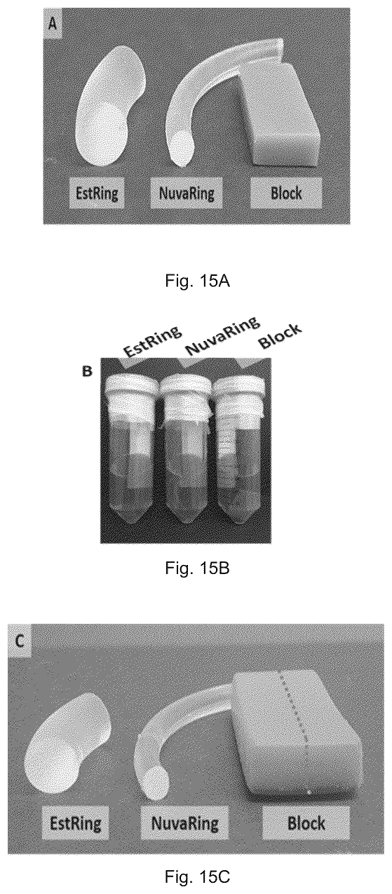

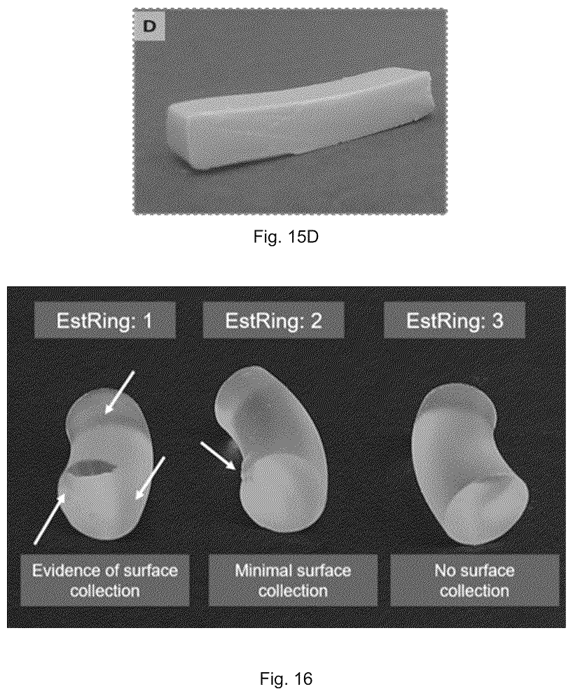

To aid in the understanding and explanation of post-loading, it was necessary to take a step back and utilize a dye to visually track the penetration of the drug. Therefore, rhodamine B (RhB) was used in place of the model drug in the post-loading process using previously optimized parameters. As shown in FIG. 15, the expected results for loading with RhB was a complete color change from the grey SIL 30 to a pinkish hue. Release profiles of post-loaded parts indicate absorption rather than adsorption therefore it was expected that cross-sections of the RhB-loaded parts will contain evenly distributed dye.

### Example 10

### Visualizing Post-Loading as a Function of Material

The SIL 30 resin, due to its unique crosslinking process, was hypothesized to react differently to the post-loading process compared to standard IVR materials such as silicone and EVA. Therefore, as a proof of concept, post-loading was conducted as a function of material in an 80 μg/mL RhB/MeOH solution using previously optimized loading parameters. Materials included placebo EstRing sections, NuvaRing sections, and SIL 30 4 mm blocks. All samples were sectioned to have similar masses with initial materials shown in FIG. 15A. A representative image of the samples during the post-loading process is shown in FIG. 10B. Images of the samples immediately following removal from the post-loading solution are shown in FIG. 15C. It was observed that the NuvaRing had negligible incorporation of RhB. The Estring sections were observed to have a thin layer of surface adsorption that easily wiped away with a ChemWipe upon removal from the post-loading solution. Cross-sections of these samples were indiscernible from whole sections. Conversely, the sectioned SIL 30 block, shown in FIG. 15D, yielded homogenous absorption of RhB throughout the structure.

Parts were quantified for gel fraction and degree of swelling following the post-loading process in RhB, with results shown in Table 12 as a function of material type. It should be noted that in this case gel fraction represents the ratio of the dry mass to initial mass as the post-loading process is not a true soluble fraction removal. The degree of swelling was calculated using swollen and dry masses of the parts. Both the NuvaRing section and Estring section yielded minimal gel fractions and minimal swelling. The post-loading process relies on swelling the structure to incorporate API, and therefore these results concur with the lack of visually observable RhB on the parts. Conversely, the SIL 30 block yielded a lower gel fraction on par with previous results and a high degree of swelling, corroborating the intensity of RhB observed in the swollen part.

TABLE 12
Post-Loading metric analysis for parts post-loading in RhB/MeOH as a
function of material type. Gel fraction is denoted with an asterisk as it
does not represent a true removal of the soluble fraction but rather
represents the ratio between the dry mass and the initial mass. Degree
$of swelling was calculated as ( \frac{Swollen Mass - Dry Mass}{Dry Mass} ) \times 100 \% .$
Averages and standard deviations represent n=3 samples per material.
Percent relative standard deviation is shown in italics below.

| Sample Type | Gel Fraction* | Degree of Swelling |
|---|---|---|
| Estring Section | 1.01 ± 0.27 | 5.12 ± 28.31 |
| | 26.87% | 553.46% |
| NuvaRing Section | 1.01 ± 0.03 | 3.94 ± 0.31 |
| | 2.70% | 7.82% |
| Diffusion Block | 0.91 ± 0.02 | 292.84 ± 6.97 |
| (4 mm) | 2.35% | 2.38% |

The variability in the Estring sample was concerning. It was previously observed that the RhB, following removal from the post-loading solution, was easily wiped away, suggesting an adsorption process. Previous experiments with EstRing sections in MeOH found a gel fraction of 0.97±0.00 and a degree of swelling of 2.89±0.28%. These previous observations of low variability clash with the present experiment. All three Estring sections following post-loading are shown in FIG. 16 where the differences in surface incorporation of RhB can be easily seen. This adsorption of API onto the Estring surface visually confirms an effect previously noted with EstRing sections. Previous experiments in post-loading Estring sections with β-estradiol yielded a powdery surface that was easily wiped away. Release studies found a burst release effect resulting in 90% cumulative release in 4 days. RhB-loaded samples are currently undergoing extraction and RhB quantification will be done via fluorescence detection to confirm observations.

Experiments with post-loading as a function of material type indicate that RhB in MeOH serves as a useful tool to visualize the post-loading process. Commercially available materials such as silicone and EVA were found to be incompatible with the post-loading process. The Estring sections at best exhibit surface adsorption of API that was unpredictable and wildly variable. Conversely, the SIL 30 blocks exhibited complete and homogenous incorporation of RhB, as shown in the cross-section. Collectively, these findings corroborate what was previously observed in post-loaded release studies. These data suggest that SIL 30, likely due to its crosslinking process, is uniquely amenable to the post-loading process developed.

### Example 11

### Visualizing Post-Loading as a Function of Loading Concentration

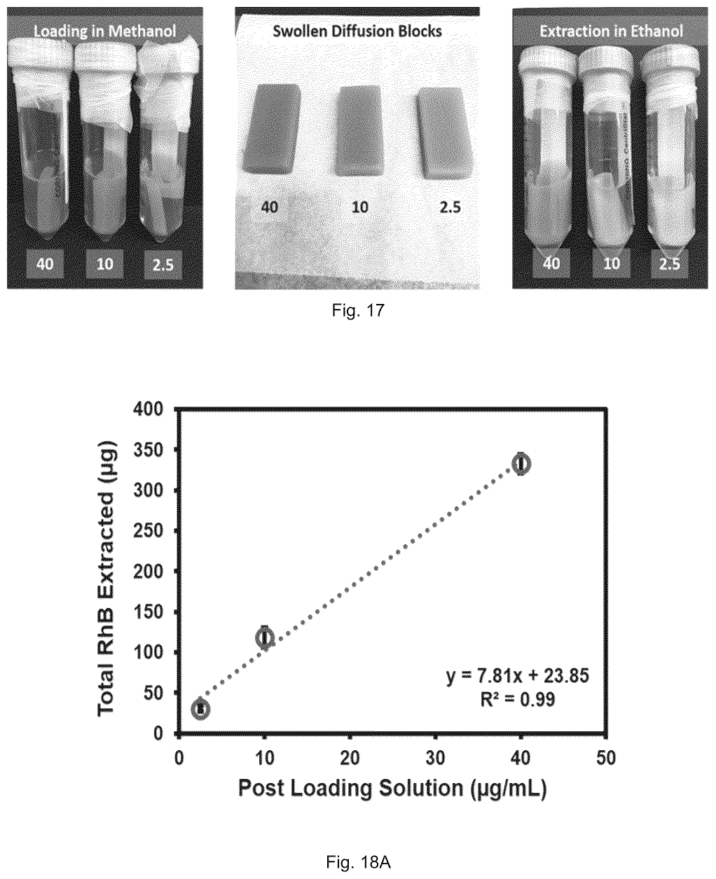

To further investigate the visualization of post-loading, SIL 30 blocks were post-loaded as a function of RhB concentration in MeOH. The process is shown in FIG. 17, where blocks were treated with 20 mL of a four-fold dilution series of RhB in MeOH. Concentrations used were 40, 10, and 2.5 μg/mL. Blocks are shown during the loading process, immediately following removal from solution and during the extraction process. The RhB enables the visual tracking of the concentration of the solutions. It can be observed that both the intensity of the post-loaded structures and extraction solutions track with the dilution series, as expected.

It should be noted that the initial intention of these samples was to image using fluorescence microscopy. Therefore, initial RhB concentration values were conservative due to concerns of saturating the detector. However, the uptake of RhB was so clearly visual and worked so well, that fluorescence detection would not have been compatible. Therefore, samples were assessed during the post-loading process and the RhB extracted for quantification.

Metrics of the blocks treated as a function of post-loading solution concentration were tracked enabling the calculation of gel fraction* and degree of swelling. As previously mentioned, the gel fraction in this case merely represents the ratio of dry mass to initial mass. Given that both dimensional and mass metrics are tracked during the loading process, degree of swelling can be calculated both on the primary azimuthal axis's as well as traditionally with mass. These values are shown in Table 13 for each loading concentration as well as pooled. The homogenous absorption of RhB was observed in the nearly uniform gel fractions*. The minimally variable degree of dimensional and mass swelling suggests that increasing RhB concentration does not affect the post-loading mechanism of SIL 30. That is, the blocks behave similarly regardless of loading concentration.

TABLE 13
Post-Loading swelling metrics for SIL 30 blocks loading with RhB in MeOH
as a function of loading concentration. Gel fraction is denoted with an
asterisk as it does not represent a true removal of the soluble fraction
but rather represents the ratio between the dry mass and the initial mass.
Degree of swelling was calculated in terms of directional swelling (X, Y,
and Z axis) as well as total mass swelling. Average and standard deviations
for each concentration represent n = 3 samples. All samples were pooled
and a combined average calculated, representing n = 9 samples.

| RhB Loading [C] | Residual | Degree of Swelling (%) | | | |
|---|---|---|---|---|---|
| μg/mL | Fraction* | Z | Y | X | Mass |
| 40 | 0.92 ± 0.00 | 68.8 ± 4.5 | 64.8 ± 1.8 | 72.0 ± 2.9 | 272.3 ± 11.7 |
| 10 | 0.92 ± 0.01 | 66.8 ± 3.3 | 63.7 ± 3.0 | 67.5 ± 3.0 | 268.6 ± 24.1 |
| 2.5 | 0.92 ± 0.00 | 70.9 ± 1.5 | 65.0 ± 1.3 | 67.3 ± 2.4 | 273.2 ± 5.6 |
| Combined | 0.92 ± 0.00 | 68.9 ± 3.4 | 64.5 ± 1.9 | 68.9 ± 3.3 | 271.4 ± 13.8 |

Finally, blocks loaded as a function of RhB concentration were extracted in EtOH and 200 μL aliquots were analyzed for fluorescence via plate reader. A standard curve of RhB was prepared in EtOH. Each standard and sample were run in triplicate. The quantification of RhB was done using the averaged standard curve (R²=0.992, EtOH evaporation contributed to error). The results are shown in FIG. 18 for total RhB extraction (A) and weight percent RhB extraction (B). Each point represents triplicate run of n=3 samples and therefore is the average of n=9 results. Standard deviation was calculated accordingly. As suspected, there was a linear relationship between loading concentration and RhB incorporation, FIG. 18A. A larger dilution series would further explore the relationship between loading concentration and uptake however the purpose of this experiment was to visualize loading. The same can be said for FIG. 18B where the weight percent loading is linearly dependent on the post-loading solution concentration but the total values are unimpressive compared to results using β-estradiol and progesterone. Recall, the original intention was to utilize fluorescence detection and therefore conservative solutions were prepared (they were still ridiculously pink).

### Example 12

### Loading as a Function of Diffusion Distance

A simplified system of diffusion blocks was investigated for properties during the post-loading process. Three types of block preparation were investigated: those treated to a simulated loading process in the absence of drug, those pre-loaded with β-estradiol, and those post-loaded with β-estradiol. Swelling properties were compared between simulated and actual post-loaded blocks. Quantification metrics of pre-loaded and post-loaded blocks were evaluated and release profiles in SVF were obtained.

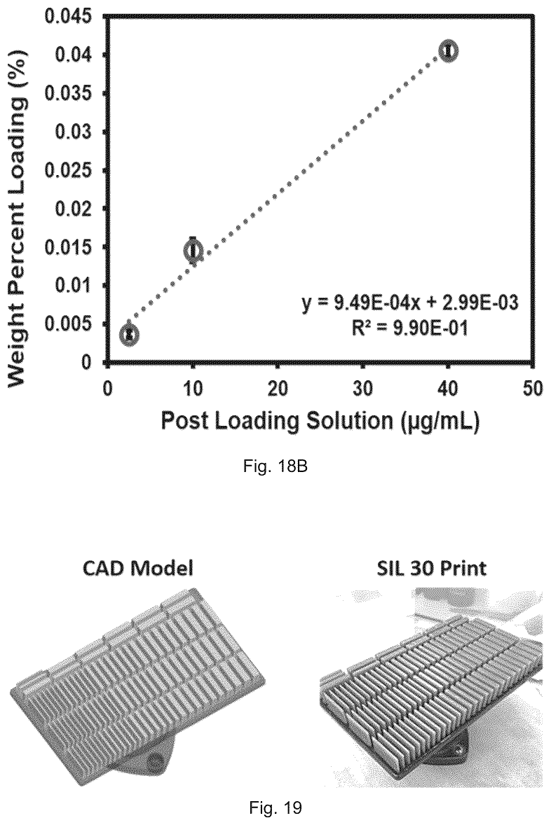

The diffusion distance within the unit cell of the IVR may dictate in part the rate of release. The overarching goal of this research was to obtain a better understanding of the contribution of diffusion distance on release rate to enabled more targeted designs. Therefore, a simplified system involving diffusion blocks fabricated as a function of distance was developed. Shown in FIG. 19 is the CAD model in the Carbon UI alongside the fabricated structures in SIL 30. Distances were specified at 4.0, 3.0, 2.0, 1.0, and 0.5 mm. These structures were fabricated both unloaded and pre-loaded with 4.0 wt. % β-estradiol.

### Example 13

### Swelling as a Function of Distance and Solvent

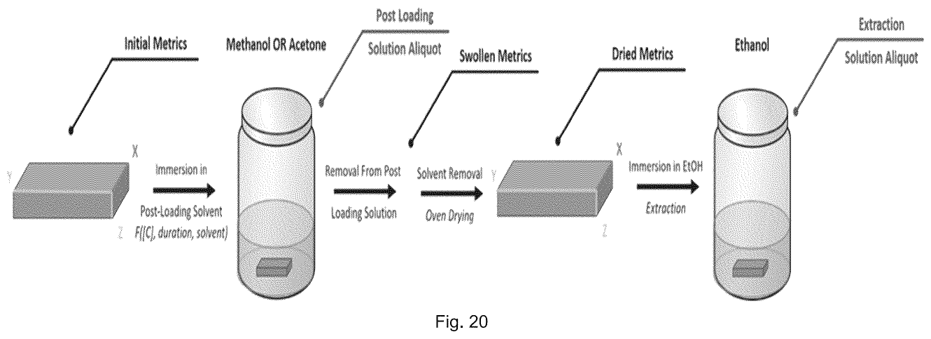

It was necessary to establish the swelling characteristics of blocks fabricated as a function of distance in the absence of drug to determine baseline behavior. Outlined in FIG. 20 is the experimental flow designed to mimic the post-loading process. In short, blocks are treated to solvent for 24 hr and dried using optimized parameters. Blocks are then treated to a 72 hr. extraction process. Marked at various points along the experimental flow are where metrics and aliquots were taken. As with previous results involving blocks, the known dimensions of the structure enable check point measurements throughout the process. Two solvents were used, methanol and acetone, given their utility in the post-loading process. Each solvent type and block distance was run in triplicate. The azimuthal axis' measured are described in FIG. 20 where the Z axis is the changing ‘diffusion distance’.

The quantification metrics collected throughout the experiment including dimensional and mass information are compiled in Tables 14-16 for initial, swollen, and dried states, respectively. Average and standard deviation values were calculated within each condition. For the X and Y dimensions, which were not varied during fabrication, the entire series for each solvent was compiled, resulting in a ‘combined’ value, shown in italics. The initial values in Table 14 include a percent deviation in Z from CAD calculation. This value describes the difference in the Z axis of the fabricated structure from the specified CAD dimension. It can be seen that as diffusion distance decreases, both deviation and variability increase. Because these blocks were taken from the same batch and then divided, the variability and deviation are expected to be similar for each solvent group. Accounting for error finds this to be true. Therefore, it can be concluded that the sample sets, while deviating and variable, do so similarly and with a predictable trend.

TABLE 14
Initial metrics of blocks as a function of diffusion distance. Dimension measurements
in mm are based on directions defined in FIG. 2. Percent deviation in the
Z axis from the specified dimensions in CAD. Dimension values in X and Y
are combined per solvent condition. Average and standard deviation values
represent n = 3 per distance and n = 15 per combined solvent.

| | Block | | | | | Z Deviation |
|---|---|---|---|---|---|---|
| Solvent | Type | X (mm) | Y (mm) | Z (mm) | Mass (mg) | From CAD (%) |
| Methanol | 4.0 | 9.80 ± 0.08 | 20.07 ± 0.06 | 4.17 ± 0.04 | 902.97 ± 5.93 | 4.33 ± 0.95 |
| | 3.0 | 9.91 ± 0.05 | 19.99 ± 0.02 | 3.16 ± 0.03 | 686.70 ± 12.58 | 5.33 ± 0.88 |
| | 2.0 | 9.95 ± 0.05 | 19.89 ± 0.04 | 2.11 ± 0.04 | 458.87 ± 4.14 | 5.67 ± 1.76 |
| | 1.0 | 9.96 ± 0.11 | 19.71 ± 0.17 | 1.22 ± 0.03 | 264.93 ± 22.57 | 21.67 ± 3.21 |
| | 0.5 | 9.87 ± 0.10 | 19.71 ± 0.10 | 0.74 ± 0.07 | 145.67 ± 12.48 | 48.00 ± 14.00 |
| Combined Methanol | | 9.90 ± 0.09 | 19.87 ± 0.17 | — | — | — |
| Acetone | 4.0 | 9.84 ± 0.07 | 20.07 ± 0.01 | 4.13 ± 0.03 | 894.23 ± 7.11 | 3.25 ± 0.66 |
| | 3.0 | 9.84 ± 0.05 | 19.96 ± 0.03 | 3.12 ± 0.03 | 683.80 ± 15.37 | 4.00 ± 0.88 |
| | 2.0 | 9.96 ± 0.06 | 19.93 ± 0.05 | 2.13 ± 0.03 | 467.87 ± 13.32 | 6.33 ± 1.26 |
| | 1.0 | 9.95 ± 0.08 | 19.77 ± 0.06 | 1.24 ± 0.02 | 268.47 ± 9.26 | 24.33 ± 1.53 |
| | 0.5 | 9.84 ± 0.06 | 19.67 ± 0.14 | 0.76 ± 0.05 | 145.83 ± 8.85 | 51.33 ± 9.24 |

| | | | | | |
|---|---|---|---|---|---|
| Combined Acetone | 9.89 ± 0.08 | 19.88 ± 0.16 | — | — | — |

The swollen metrics were combined and calculated in a similar manner in Table 15. Again, values for the X and Y dimensions were combined within each solvent type. The blocks appear to swell slightly more in methanol than acetone however this could be a productive of part geometry. The swelling dimensions in X and Y appear to trend with the decreasing diffusion distance. Interestingly, when combined, the values indicate that the blocks, regardless of Z distance, swell similarly in the X and Y dimensions. This was encouraging as it suggests a device could potentially have multiple distances (i.e. band distances and unit cell distances) and swelling behavior would not be lopsided or uneven. It should be noted that the degree of swelling by percent decreases with decreasing diffusion distance. It is likely that this is a product of the decreasing surface area to volume ratio. Calculations are currently being conducted to test this theory.

TABLE 15
Swollen metrics of blocks as a function of diffusion distance. Dimension
measurements in mm are based on directions defined in FIG. 20. Degree of
swelling calculated from swollen and dried masses. Dimension values in
X and Y are combined per solvent condition. Average and standard deviation
values represent n = 3 per distance and n = 15 per combined solvent.

| | Block | | | | | Degree of |
|---|---|---|---|---|---|---|
| Solvent | Type | X (mm) | Y (mm) | Z (mm) | Mass (mg) | Swelling (%) |
| Methanol | 4.0 | 17.14 ± 0.03 | 33.36 ± 0.19 | 7.00 ± 0.20 | 3532.6 ± 90.7 | 347.1 ± 36.9 |
| | 3.0 | 17.11 ± 0.05 | 33.21 ± 0.30 | 5.36 ± 0.16 | 3032.4 ± 287.5 | 418.3 ± 53.7 |
| | 2.0 | 16.26 ± 0.05 | 32.08 ± 0.18 | 3.64 ± 0.19 | 1593.4 ± 26.5 | 290.8 ± 3.8 |
| | 1.0 | 15.99 ± 0.19 | 31.90 ± 0.21 | 2.06 ± 0.02 | 873.7 ± 86.9 | 275.3 ± 5.3 |
| | 0.5 | 15.50 ± 0.23 | 30.82 ± 0.12 | 1.44 ± 0.04 | 461.6 ± 35.2 | 263.3 ± 28.1 |
| Combined Methanol | | 16.40 ± 0.67 | 32.27 ± 0.98 | — | — | — |
| Acetone | 4.0 | 16.72 ± 0.14 | 32.32 ± 0.17 | 6.63 ± 0.19 | 3479.2 ± 254.3 | 356.3 ± 49.3 |
| | 3.0 | 17.28 ± 0.31 | 32.37 ± 0.16 | 4.99 ± 0.15 | 2702.5 ± 99.2 | 366.6 ± 15.8 |
| | 2.0 | 15.96 ± 0.08 | 31.25 ± 0.23 | 3.57 ± 0.11 | 1536.3 ± 83.9 | 271.1 ± 11.5 |
| | 1.0 | 15.66 ± 0.04 | 31.23 ± 0.11 | 2.14 ± 0.07 | 828.7 ± 29.9 | 249.2 ± 2.2 |
| | 0.5 | 14.37 ± 0.10 | 29.97 ± 0.13 | 1.17 ± 0.04 | 424.5 ± 19.0 | 231.1 ± 7.3 |

| | | | | | |
|---|---|---|---|---|---|
| Combined Acetone | 16.00 ± 1.04 | 31.43 ± 0.92 | — | — | — |

Finally, the dried metrics are shown in Table 16 as a function of diffusion distance. Here, percent deviation from CAD represents the degree of deviation of the dried structures from the original CAD dimensions. For methanol, the final dimensions in X are similar to the initial dimensions and the Y dimensions are collectively smaller. For acetone, both the X and Y dimensions are collectively smaller. Within error, the deviation from CAD was largely the same for both methanol and acetone.

TABLE 16
Dried metrics of blocks as a function of diffusion distance. Dimension measurements
in mm are based on directions defined in FIG. 2. Percent deviation in the
Z axis from the specified dimensions in CAD. Dimension values in X and Y
are combined per solvent condition. Average and standard deviation values
represent n = 3 per distance and n = 15 per combined solvent.

| | Block | | | | | Deviation |
|---|---|---|---|---|---|---|
| Solvent | Type | X (mm) | Y (mm) | Z (mm) | Mass (mg) | From CAD (%) |
| Methanol | 4.0 | 9.72 ± 0.06 | 19.66 ± 0.04 | 4.04 ± 0.03 | 7.92.5 ± 43.2 | 0.9 ± 0.7 |
| | 3.0 | 9.74 ± 0.09 | 19.68 ± 0.08 | 3.06 ± 0.05 | 585.6 ± 14.6 | 1.9 ± .15 |
| | 2.0 | 9.68 ± 0.05 | 19.54 ± 0.03 | 2.02 ± 0.01 | 407.7 ± 4.1 | 1.2 ± 0.6 |
| | 1.0 | 9.67 ± 0.2 | 19.48 ± 0.07 | 1.11 ± 0.01 | 232.7 ± 20.6 | 11.0 ± 1.0 |
| | 0.5 | 9.54 ± 0.11 | 19.45 ± 0.07 | 0.71 ± 0.03 | 127.4 ± 11.3 | 41.3 ± 6.4 |
| Combined Methanol | | 9.97 ± 0.10 | 19.56 ± 0.11 | — | — | — |
| Acetone | 4.0 | 9.44 ± 0.10 | 19.28 ± 0.04 | 4.06 ± 0.02 | 764.6 ± 32.2 | 1.6 ± 0.5 |
| | 3.0 | 9.36 ± 0.03 | 19.22 ± 0.03 | 3.03 ± 0.02 | 579.2 ± 6.7 | 1.1 ± 0.8 |
| | 2.0 | 9.58 ± 0.10 | 19.16 ± 0.03 | 2.07 ± 0.01 | 413.8 ± 10.3 | 3.5 ± 0.5 |
| | 1.0 | 9.44 ± 0.12 | 19.08 ± 0.05 | 1.17 ± 0.07 | 237.3 ± 7.5 | 17.3 ± 7.2 |
| | 0.5 | 9.30 ± 0.06 | 18.79 ± 0.13 | 0.78 ± 0.06 | 128.3 ± 8.2 | 55.3 ± 12.7 |

| | | | | | |
|---|---|---|---|---|---|
| Combined Acetone | 9.55 ± 0.16 | 19.34 ± 0.24 | — | — | — |

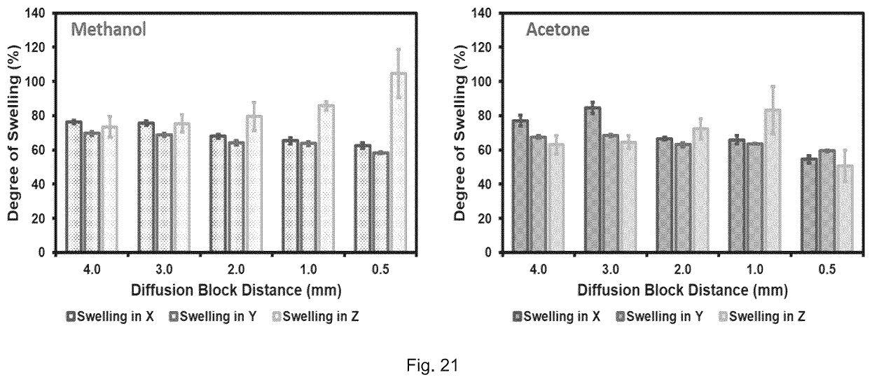

The above data can be used to calculate degrees of swelling for the X, Y and Z dimension using the following equation:

$$Degree of Swelling ( \% ) = 100 \times ( {A}_{S} - {A}_{D} / {A}_{D} )$$

where A represents values in a given dimension. These values were plotted as a function of diffusion distance shown in FIG. 21A-B for methanol and acetone, respectively. For both solvents, the larger distances appear to swell more evenly in terms of collective part expansion. For methanol, the Z dimension uptake increases with decreasing diffusion distance. For acetone, the Z dimension appears relatively constant. Interestingly, for both solvents, the variability, as shown by the error bars, spikes for the 0.5 mm blocks. As previously mentioned, these blocks both deviate from initial CAD dimensions and had the highest initial variability. This suggests that variability in the initial sample set was compounded as the post-loading process progresses.

The purpose of this study was to gage gel fraction as a function of diffusion distance with methanol as a swelling solvent. This does not represent a true soluble fraction removal as the soak time was limited to 24 hours as opposed to the previously determined 4-6 days. The calculation of gel fraction instead allows the inference of how much potential extractables can be removed from the part during the post-loading process. This is a critical metric to understand given that the parts do contain a sizable percentage of unreacted/unincorporated monomer/oligomers. Minimizing leachables remains a primary concern. Therefore, understanding what ‘comes out’ during the post-loading process can be helpful. These values are shown in FIG. 22A as a function of distance and solvent. Interestingly, the highest variable samples are the 4.0 mm blocks. This makes sense as they have the potential to have the largest presence of unreacted components that were not washed away. Excluding this set, the gel fractions are largely constant with minimal variability. It is perhaps something to note that if nothing is really gained by the 4 mm distance in terms of release kinetics or loading, then smaller distances would be preferable given the ability to more readily remove potential leachables. The degree of swelling, calculated using mass as described in Table 2 was plotted as a function of distance for each solvent and a linear fit applied, as shown in FIG. 22B. Both solvents exhibited a slight but predictable linear trend of degree of swelling as a function of diffusion distance, where the smaller distances swelled less than the larger distances. Again, this could be a product of specific surface area changes and calculations are currently being conducted to investigate further.

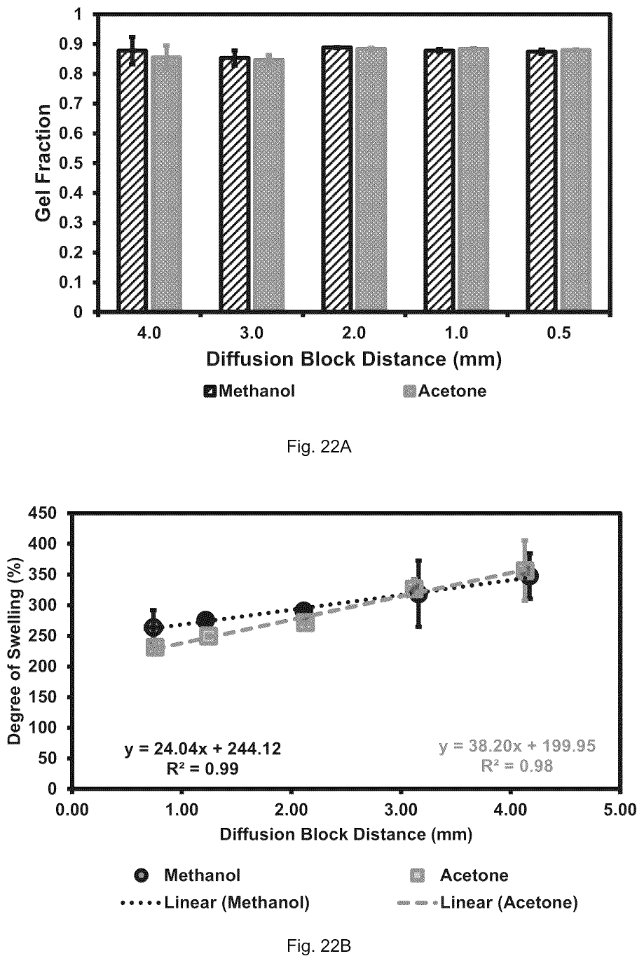

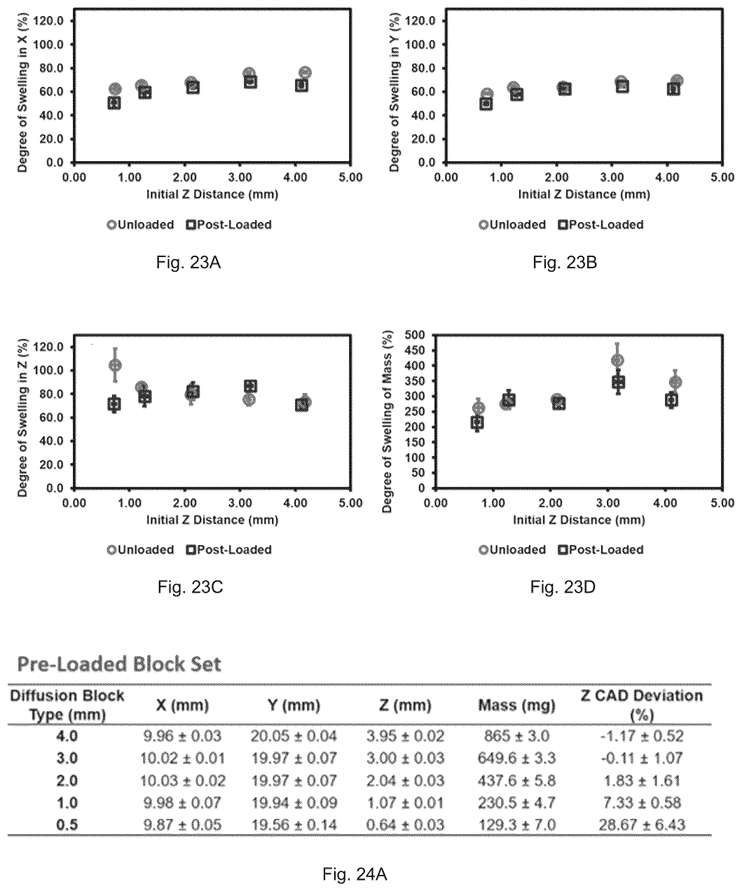

It was important to understand how the metrics obtained during the simulated loading process compared to metrics obtained during the actual post-loading of blocks as a function of diffusion distance. Shown in FIG. 23A-D are degrees of swelling in methanol for X, Y, Z and mass, respectively, comparing unloaded to post-loaded. The overarching trend suggests that there was minimal difference in the swelling behavior of these blocks in the presence or absence of drug. There was a slightly significant increase in swelling in the Z dimension for unloaded blocks over loaded blocks. Additionally, the linear trend observed for the degree of swelling in the unloaded blocks appears more obscure in the loaded blocks. Collectively, however, there appears to be minimal changes in the behavior of the blocks in the presence of a super saturated drug (16 mg/mL β-estradiol in methanol).

Finally, the gel fractions of unloaded and loaded samples were compared as a function of diffusion distance and compiled in Table 17. The percent increase in gel fraction from the presence of drug was also calculated. The gel fraction values for the loaded series are higher, which was to be expected given the calculation does not represent a true soluble fraction removal. It was interesting that the percent increase, while positive for all distances, was not constant nor does a trend emerge. Rather, the values range from 2-10%.

TABLE 17
Gel fraction by loading condition per block type in methanol.
Unloaded gel fractions refer to soluble fraction removal in
methanol in 24 hrs. Loaded gel fraction* represents soluble
fraction removal and drug incorporation in methanol in 24
hrs. Fraction increase from loading is calculated as percent
increase from unloaded to loaded. Average and standard deviation
values represent n = 4 per sample condition.

| Diffusion Block | | | Fraction Increase From |
|---|---|---|---|
| Type (mm) | Unloaded | Loaded | Loading (%) |
| 4.0 | 0.88 ± 0.05 | 0.97 ± 0.00 | 10.32 |
| 3.0 | 0.85 ± 0.02 | 0.94 ± 0.01 | 10.70 |
| 2.0 | 0.89 ± 0.00 | 0.94 ± 0.03 | 5.94 |
| 1.0 | 0.86 ± 0.00 | 0.88 ± 0.04 | 2.33 |
| 0.5 | 0.87 ± 0.01 | 0.93 ± 0.05 | 6.67 |

### Example 14

### Pre vs. Post Loading

### Dimensions and Loading

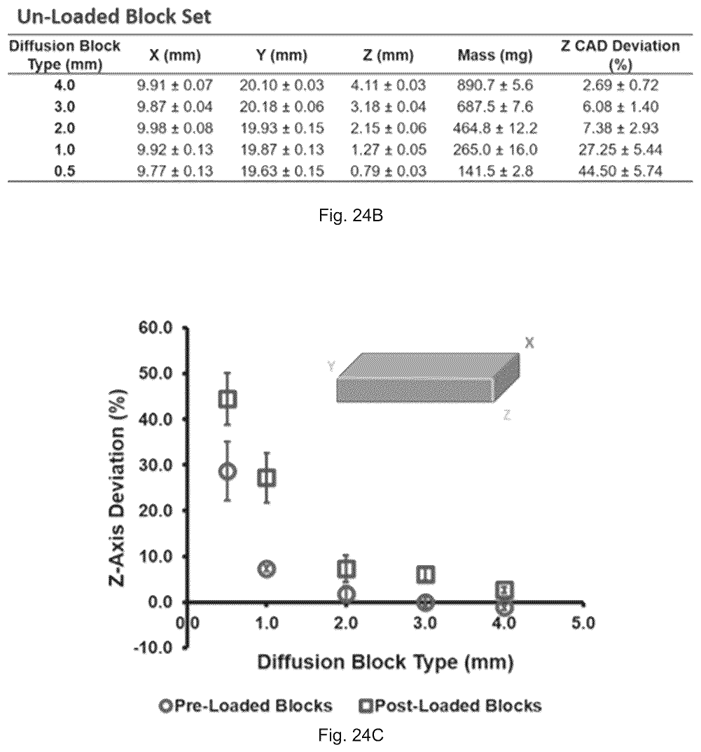

Blocks as a function of diffusion distance were fabricated both pre-loaded and unloaded (for post-processing loading). Pre-loaded blocks were fabricated with 4.0 wt. % β-estradiol. Previous post-loading experiments found 4 mm blocks could be loaded between 4-6 wt. % however β-estradiol solubility was limited in SIL 30, therefore, to be conservative, the lower loading limit was selected. Initial metric values for pre-and un-loaded blocks are shown in FIG. 24A-B. The percent deviation from CAD was plotted as a function of distance in FIG. 24C. For both series, the deviation and variability both increase with decreasing diffusion distance. The pre-loaded samples deviated less from the original CAD dimensions than the post-loaded samples. It is possible that the β-estradiol was not fully soluble in the SIL 30 and therefore remained in particulate form. Particles in resin have been shown to refract the projected light, in turn altering the dimensions of the polymerized part. This could explain the differences in dimensions from the pre-and post-loaded structures. Additionally, the partially soluble β-estradiol in the pre-loaded parts could alter the loading and release characteristics.

Unloaded diffusion blocks were post-loaded with a super saturated solution of β-estradiol in three individual batches, designated by loading round. The differences in the loading rounds are identified in Table 18. The original post-loading method utilized glass jars to prepare the samples. However, for the large number of samples used in the diffusion distance set, 50 mL falcon tubes were used in Round I. It was observed that the tubes inhibited the swelling of the blocks. Therefore, Round II and Round III were prepared according to the original protocol. Round I and Round II were used for extraction and Round III used in a release study. The gel fraction and degree of swelling metrics were pooled for each block dimension and compiled in Table 18. The compiled values represent a total of 11 samples. Gel fraction values simply represent the ratio of dried to initial mass. The degree of swelling was calculated using the previously mentioned equation. Collectively there was a decreasing trend in gel fraction and degree of swelling with decreasing diffusion distance. This was previously observed with the unloaded samples. It was interesting to see the variability, as shown by the percent relative standard deviation, increases in the Z dimension as the diffusion distance decreases. The variability was previously noted as being initially significant and therefore it can be seen that the error compounds during the post-loading process.

TABLE 18
Collective gel fraction* and swelling metrics from three rounds of post-loading as
a function of diffusion distance. Gel fraction and swelling metrics collected during
the process of post-loading by round type with specifications of preparation methods
and sample purpose described. Average and standard deviations represent n =
11 per block type. Percent relative standard deviation is shown in italics.

| | | Round II: | | Round III: | |
|---|---|---|---|---|---|
| Round I: | | Extraction | | Release | |
| Extraction | | Prepared in 60 mL Glass | | Prepared in 60 mL Glass | |
| Prepared in 50 mL Falcon | | Jars | | Jars | |
| Tubes | | (n = 4) | | (n = 4) | |
| (n = 3) | | Degree of Swelling (%) | | | |
| Block Type (mm) | Gel Fraction* | X | Y | Z | Mass |
| 4.0 | 0.97 ± 0.01 | 68.6 ± 4.4 | 64.2 ± 3.0 | 72.6 ± 6.7 | 302.0 ± 23.8 |
| | 0.69% | 6.43% | 4.66% | 9.29% | 7.88% |
| 3.0 | 0.95 ± 0.03 | 69.6 ± 2.5 | 65.4 ± 1.9 | 77.9 ± 8.6 | 338.8 ± 32.7 |
| | 2.78% | 3.56% | 2.94% | 10.99% | 9.65% |
| 2.0 | 0.94 ± 0.03 | 64.4 ± 2.1 | 63.8 ± 2.0 | 71.8 ± 10.7 | 285.1 ± 16.8 |
| | 2.71% | 3.31% | 3.16% | 14.92% | 5.90% |
| 1.0 | 0.91 ± 0.06 | 59.3 ± 2.5 | 57.8 ± 1.9 | 74.5 ± 15.4 | 272.0 ± 30.6 |
| | 6.39% | 4.27% | 3.22% | 20.65% | 11.24% |
| 0.5 | 0.91 ± 0.07 | 54.1 ± 3.6 | 52.1 ± 2.9 | 65.4 ± 20.2 | 264.3 ± 52.6 |
| | 8.07% | 6.59% | 5.58% | 30.84% | 19.91% |

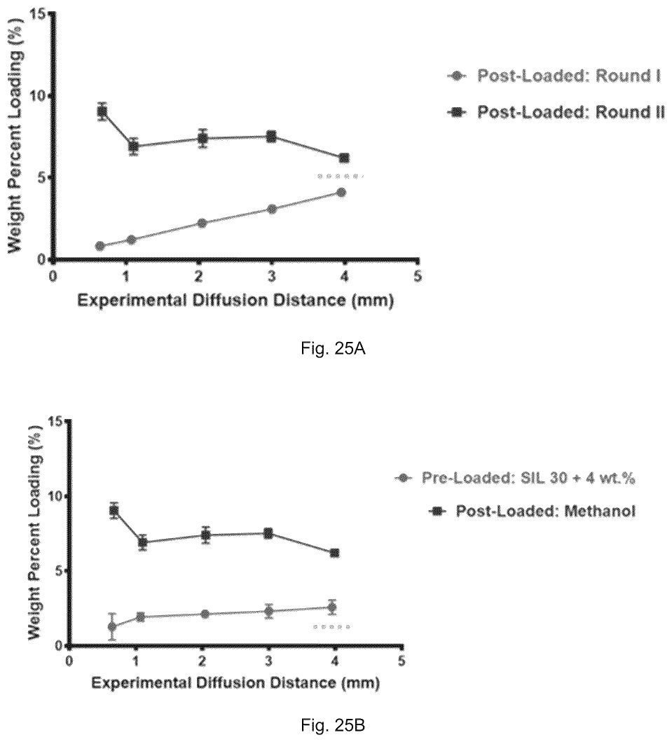

Both pre-loaded and post-loaded blocks were extracted in 20 mL of EtOH for 72 hours. Aliquots were taken and analyzed via HPLC. The weight percent loadings are shown in FIG. 25. The post-loading rounds described in Table 18 with loading Round I and II shown in FIG. 25A. The effect of the loading vessels can be seen in the incorporation of β-estradiol. The inhibition of the swelling behavior in the 50 mL falcon tubes resulted in an overall weight percent loading. Therefore, for the sake of consistency between previous results and subsequently prepared samples, loading values obtained in Round II were used for all calculations.

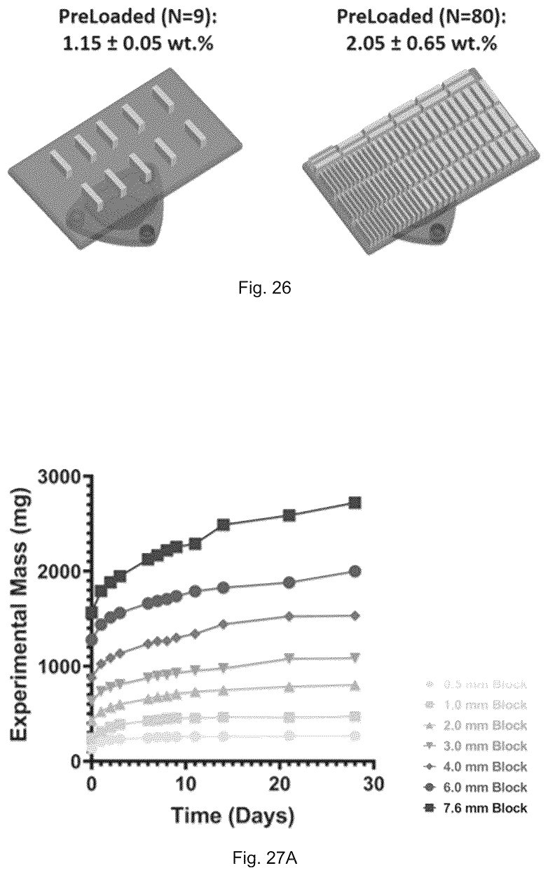

Weight percent loadings between pre-and post-loaded blocks are compared in FIG. 25B. The weight percent loadings of the pre-loaded blocks were found to be both variable and far lower than the specified 4 wt. % loading. As previously suggested, the β-estradiol was not fully dissolved in the SIL 30, resulting in deviations from the CAD specified dimensions. This partial solubility was further compounded by the ‘crowded’ fabrication environment of the blocks, as illustrated in FIG. 26. A previous pre-loaded set was fabricated with 1.0 wt. % β-estradiol loading in the configuration shown on the left. The resulting extracted amount was very close to the specified amount. Contrastingly, the diffusion distance set, shown on the right, placed parts close together. The crowded environment resulted in uneven consumption of the two parts of the resin. It was hypothesized that the β-estradiol was preferentially soluble in one of the components and therefore, any change in the resin makeup will result in a change in solubility. This was previously observed with IVRs fabricated in a sequential manner where the IVRs printed earlier in the series differed greatly in loading than those printed later on, again suggesting a drug solubility change. This reasons for this are further elaborated in Progress Summary Part III. However, it should be noted that this crowded environment resulted in both a deviation from the originally specified loading and an increase in variability that was not previously seen in an ‘uncrowded’ environment. The SIL 30 resin aliquot collected during the pre-loaded diffusion distance is expected to contain a much higher concentration of β-estradiol than the parts.

### Example 15

### Swelling Analysis of Acetone-Treated Blocks in Simulated Vaginal Fluid (SVF)

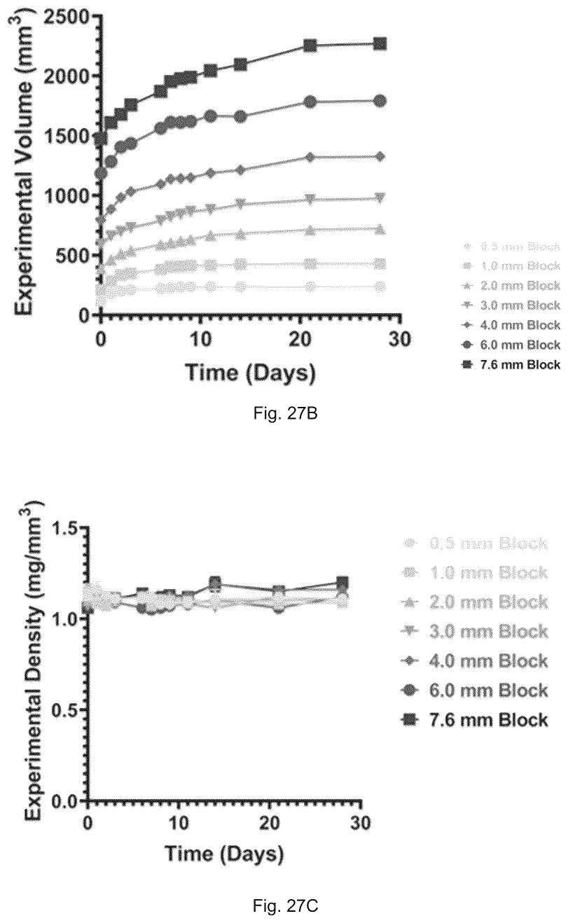

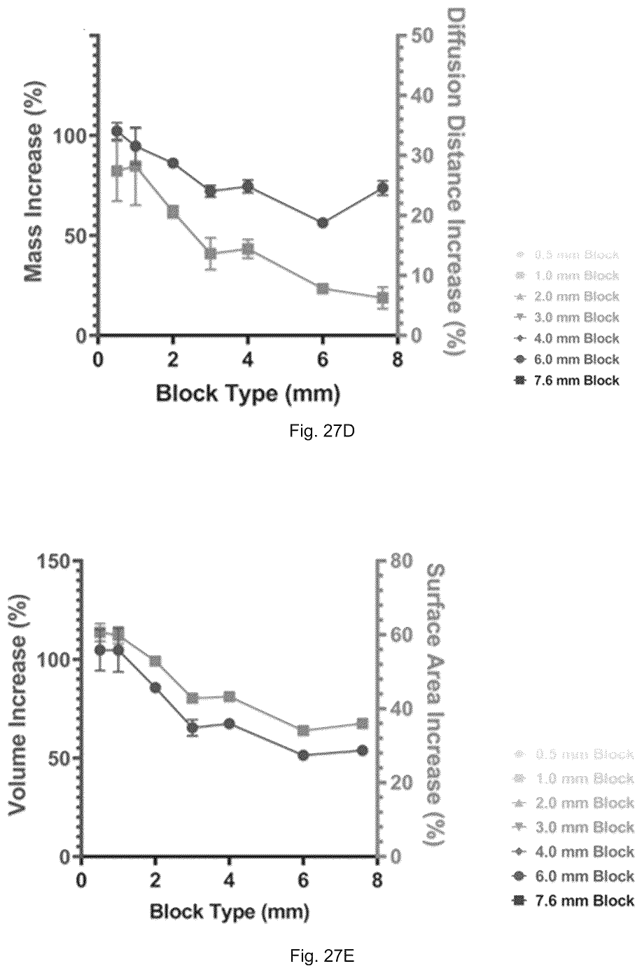

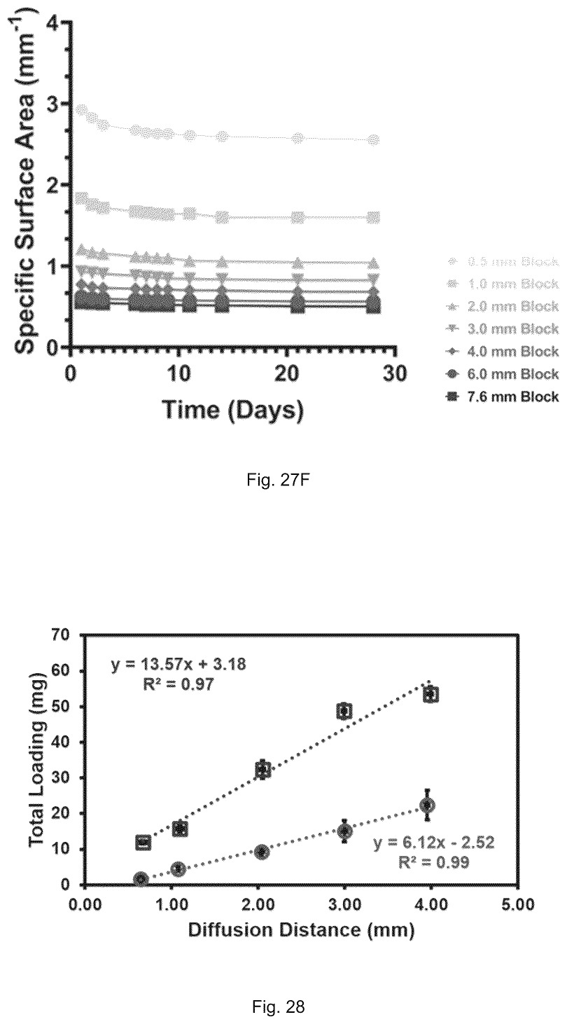

A proposed utility of the post-loading method is as a drug incorporation process for 3D-printed intravaginal rings (IVRs). Given the matrix can be purposely swollen to allow for drug uptake, it was necessary to investigate the swelling behavior of the SIL 30 material under relevant in vitro release conditions. All block types (0.5-7.6 mm, n=3 of each) were immersed in acetone for 24 hrs, removed and allowed to air dry, mimicking the proposed post-loading process in the absence of API. Once dried, metrics (mass and dimensions) were taken, and blocks were immersed in simulated vaginal fluid (SVF) at 37° C. Blocks were tracked daily for the first two weeks of exposure and weekly following for 28 days. Given the aqueous nature of the SVF, comparable swelling to acetone or methanol was not anticipated, however because of the unique curing conditions of the resin and the elevated temperature, some swelling was expected. The experimental mass and calculated volume of the blocks as function of exposure time in SVF are shown in FIGS. 27A and B, respectively. For all block types, mass increased with increasing exposure time however for the smaller diffusion distances (<4.0 mm), there appears to be a maximum threshold that was reach by Day 14, resulting in a plateau for the remainder of the experiment. This was also observed in the volume calculation. Conversely, the larger 6.0 and 7.6 mm blocks appear to continually, if minimally, expand in the presence of SVF. The collection of mass and calculation of volume enabled the calculation of block density as a function of time (FIG. 27C), which was observed to be stable at 1.11±0.03 mg/mm³ regardless of block type or immersion duration. This suggests that the uptake of SVF is uniform, as was observed with the swelling in organic solvents.

To assess the extent of the swelling in SVF, percent increases were computed for each block type from day 0 (initial) to day 28. The percent mass increase and diffusion distance (X dimension) are shown in FIG. 27D and demonstrate a roughly linear relationship between increase and block type. The smaller distances (0.5 and 1.0 mm) swelled the most both in terms of relative mass and distance which can be expected given the high relative surface area. Conversely, the larger 6.0 and 7.6 mm blocks exhibited much lower mass increase (<10%) as they have much lower SSA values. A similar trend was observed looking at the percent increase in volume and surface area (FIG. 27E). It should be noted that because of the differences in volume and surface area, these trends cannot be directly extended to the behavior of solid toroidal SIL 30 rings in SVF. The calculation of volume and surface area allowed for the computation of SSA as a function of block type and immersion duration, shown in FIG. 27G. These data were fitted with logarithmic curves by block type with associated coefficients of determination shown, all greater than 0.94, indicating a good fit. The smaller block distances were observed to have the largest SSA and subsequent SSA decrease in the presence of SVF. Interestingly, these fitted curves appear to overlap with increasing block distance, as evidence by the m and b parameters in the associated equations. This suggests that as the block increases, minimal relative dimensional changes are observed in the presence of SVF. Additionally, that these dimensional changes can be predicted by diffusion distance and starting SSA.

Finally, total loading values of β-estradiol were determined as a function of diffusion distance for both pre-and post-loaded blocks. These values are compiled in FIG. 28. A linear fit was applied to both sets with the R² values shown. Given the higher weight percent loading of the post-loaded blocks, it was not surprising to observe an overall larger incorporation of β-estradiol over the pre-loaded blocks. Both sets appear to be reasonably predictive in the total loading as a function of distance. This was to be expected as the part mass increases with distance.

### Example 16

### Release and Leachables

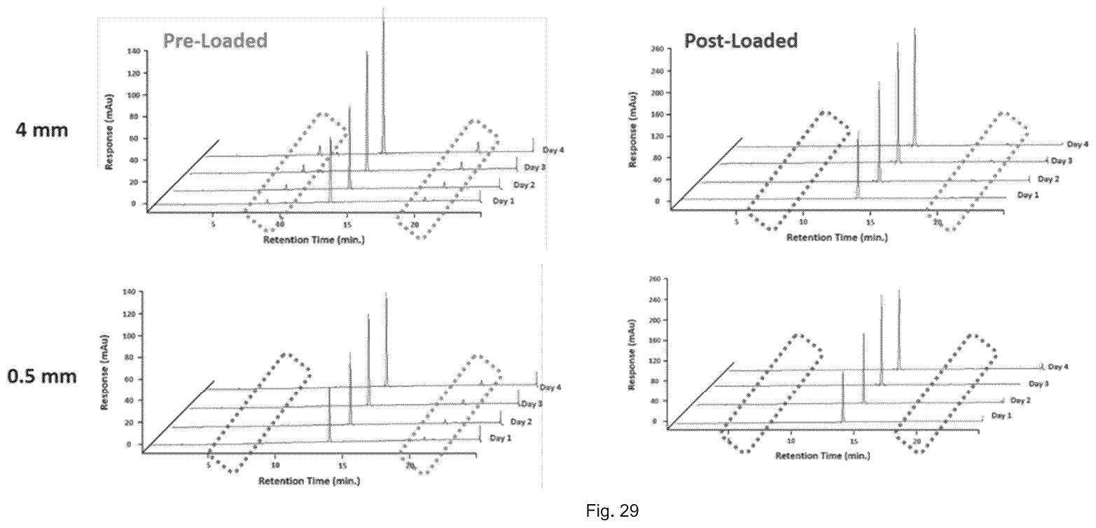

Blocks prepared both by pre-and post-loading methods were evaluated for release characteristics in simulated vaginal fluid S(VF). Blocks were placed in 60 mL of SVF and 1 mL aliquots taken as a function of time. Aliquots were analyzed for β-estradiol via HPLC. Representative chromatograms obtained are shown for both pre-and post-loaded samples in FIG. 29. Two block types are represented, 4.0 mm and 0.5 mm. Previous release studies have identified two known leachables at 8.7 min. and 20.6 min, denoted as leachable #1 and #2, respectively. The presence of the leachables were outlined in a dashed green line and absence in a dashed red line. Previous results found that for the pre-loaded samples, leachable #1 decreased with time and leachable #2 remained constant, or on par with the diffusion of the drug. For the pre-loaded samples, both leachables are present for the 4.0 mm sample, concurring with previously obtained results. Interestingly, leachable #1 was absent entirely from the 0.5 mm series. For the post-loaded samples, leachable #1 was found to be absent from the release profile, as with the pre-loaded 0.5 mm series. For the post-loaded 0.5 mm series, neither leachable was detected about the 5 mAu cutoff for noise. It has been previously suggested and supported by release data that the post-loading process removes leachables. It has been shown that larger distances have lower gel fractions. The remaining soluble fraction was the likely source of the leachables. Therefore, smaller diffusion distances are more amenable to reducing leachables because of the higher gel fractions. Additionally, this may suggest that leachable #1 was more related to unincorporated resin and leachable #2 to an unreacted resin component. Current investigation is being conducted to quantify leachables against Part A and to quantify potential resin components, specifically the photoinitiator (L-TPO).

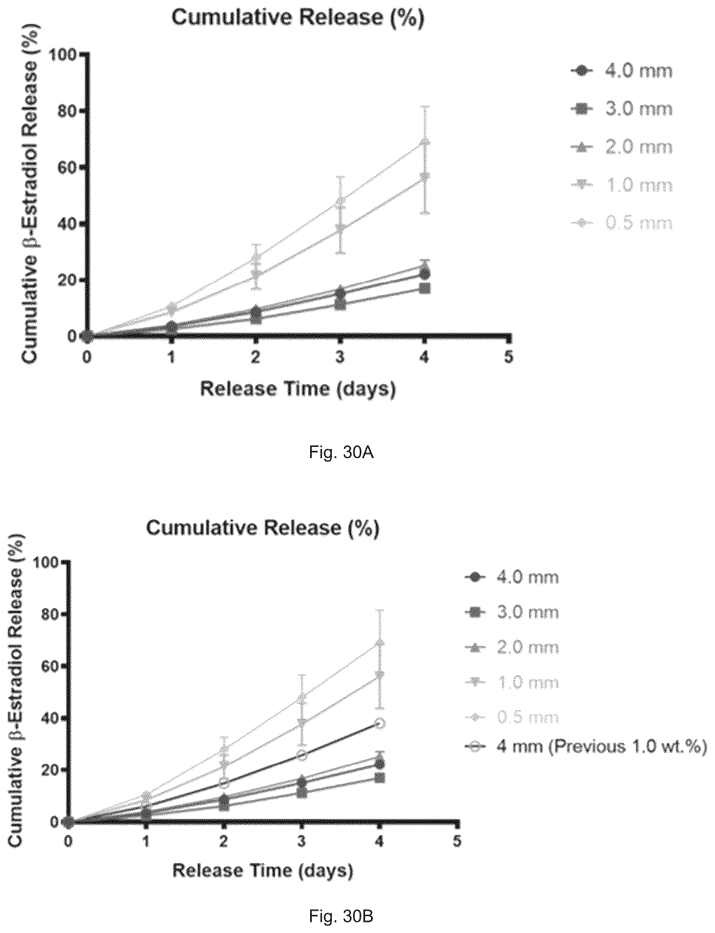

The release profile for the first four days was obtained for pre-loaded samples, shown in FIG. 30A. It can be seen that the high variability in the sample loading resulted in compounded variability in the release. Very rapid release additionally supports the theory of the presence of particulate β-estradiol, which would preferentially collect on the surface of the part resulting in a burst release. Statistical analysis was conducted on the Day 4 values and it was found that the 4.0, 3.0, and 2.0 values were statistically different from the 1.0 and 0.5 mm values, but that there were not distinguishable beyond that. Comparing the release characteristics to a previously obtained 1.0 wt. % of a 4.0 mm block shows a difference in release behavior, particularly in the variability. Differences in loading have been previously observed to affect release however, it is difficult to say whether the discrepancy is cause by loading amount, β-estradiol insolubility, or both. The remaining pre-loaded sample aliquots are currently being analyzed to determine the complete release profile and calculate corresponding zero order release metrics.

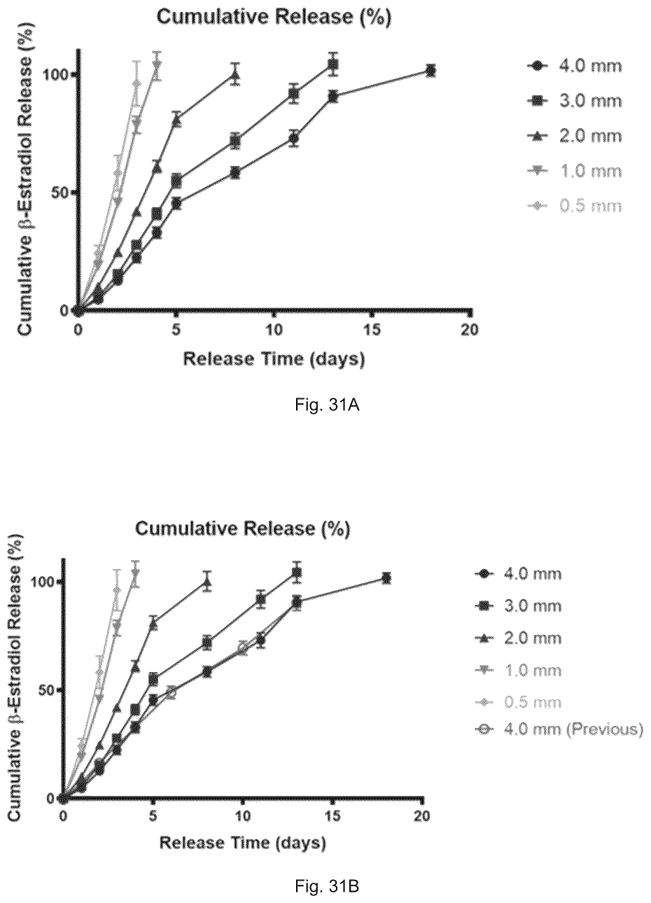

The release profiles were obtained for post-loaded samples, shown in FIG. 31A and Table 19. Percent cumulative release was shown as a function of diffusion distance and each sample set was tracked until complete release was achieved. The post-loaded samples exhibited release kinetic dependence on diffusion distance, which was extremely promising. As hypothesized, the smaller distances released faster than the larger distances. Release was compared to a previously collected sample of a 4.0 mm prepared in MeOH, shown in red in FIG. 31B. It can be seen that the values line up fairly nicely. This suggests the effect is reproducible, the selection of Round II loading values to be correct, and that complete release can be achieved. The zero order table as a function of distance is currently being prepared. As with the pre-loaded series, statistical analysis was conducted comparing the completion points of each diffusion distance. Each distance was found to be unique and statistically significant. Comparison of day to day release found some overlap, particularly between the 3.0 mm and 4.0 mm series and the 0.5 mm and 1.0 mm series in the initial portion of the release profile. Current investigation is being conducted on a more thorough statistical testing method (waiting to get SAS JMP software, free through UNC). Collectively, these data suggest that release duration and rate can be tuned by diffusion distance.

TABLE 19
In vitro release kinetics of β-estradiol as a function of diffusion distance.

| Block | | | Total | | | Drug release | Drug release |
|---|---|---|---|---|---|---|---|
| Type | Experimental | | Drug per | Burst at | Burst at | at zero order | at zero order |
| (mm) | Distance (mm) | Wt. % of Drug | Block (mg) | 24 hr. (%) | 24 hr. (mg) | (%/week) | (mg/week) |
| 4.0 | 3.99 ± 0.02 | 6.21 ± 0.23 | 53.53 ± 2.11 | 4.68 ± 0.86 | 2.51 ± 0.46 | 6.14/2.6 | 3.29/2.6 |
| 3.0 | 2.99 ± 0.02 | 7.52 ± 0.33 | 48.79 ± 2.09 | 5.64 ± 0.44 | 2.75 ± 0.21 | 7.47/1.9 | 3.64/1.9 |
| 2.0 | 2.05 ± 0.02 | 7.40 ± 0.55 | 32.38 ± 2.47 | 10.09 ± 0.68 | 3.27 ± 0.22 | 13.42/1.2 | 4.34/1.2 |
| 1.0 | 1.10 ± 0.04 | 6.91 ± 0.50 | 15.74 ± 1.28 | 18.96 ± 0.83 | 2.98 ± 0.13 | 31.69/0.7 | 5.0/0.7 |
| 0.5 | 0.67 ± 0.04 | 9.05 ± 0.52 | 11.92 ± 0.47 | 24.28 ± 3.41 | 2.89 ± 0.41 | 35.97/0.6 | 4.3/0.6 |

### Example 17

### Post-Loading with Model Drugs

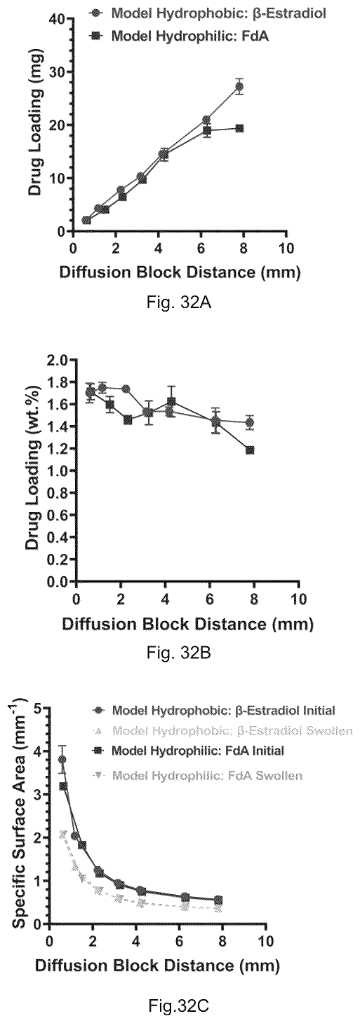

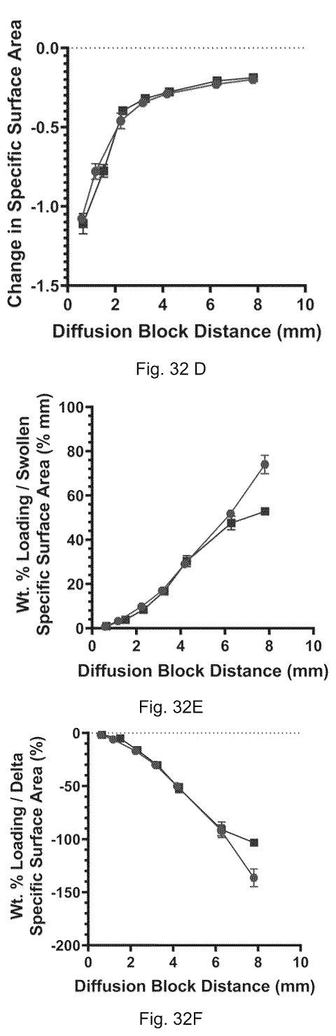

The post-loading process was investigated by incorporating small molecule model drugs or drug surrogates into the SIL 30 matrix. Two model drugs were selected: β-Estradiol (β-Est) a hydrophobic (log p of 3.75) hormone used in the treatment of menopause and commercially available in an IVR (EstRing) and 2′fluoro-2′-deoxyadenosine (FdA), a hydrophilic (log p of −0.57) analog to the nucleoside reverse transcriptase inhibitor (NRTTI) Islatravir. Blocks of all types were immersed into acetone solutions containing 5 mg/mL of either β-Est or FdA for 24 hrs. Blocks were then extracted using two cycles of acetone to determine total drug loading, shown in FIG. 32A as a function of experimental block distance. Both model drugs load similar amounts roughly linearly proportional to block distance. There is, however, a clear plateauing effect with the FdA-loaded 7.6 mm blocks. While it has been demonstrated that these blocks may not fully swell in 24 hrs, that does not explain the difference between hydrophobic and hydrophilic uptake. It should be noted that the saturation solubilities of the model drugs in acetone are 22 mg/mL and 9.5 mg/mL for β-Est and FdA, respectively. With a post-loading solution concentration of 5 mg/mL, the hydrophobic β-Est was loaded at a concentration much lower relative to the maximum possible than the hydrophilic FdA. Additionally, the SIL 30 matrix contains a hydrophobic backbone, which is why organic solvents swell the polymer and remove the soluble fraction so effectively. Therefore, the acetone for the FdA loading is required to both solubilize a relatively large amount of FdA as well as swell the SIL 30 matrix. It is possible we are observing maximum loading effects for the model hydrophilic drug.

To investigate model drug loading further, the weight percent loading per block type was investigated. In FIG. 32B, loading normalized by mass (%) is shown as a function of block type and displays a general liner correlation with decreasing wt. % loading for increasing block distance. Using a similar analysis as shown in FIG. 32G, the SSA values were computed for both the initial and swollen states of the blocks, shown in FIG. 32C. The swelling behavior of the blocks does not appear to be altered by the presence of either hydrophobic or hydrophilic model drug, as alluded to in FIG. 14E, and the exponential relationship of the swollen specific surface area (S-SSA) is maintained. From these data, it is possible to compute the change in SSA or Delta SSA, shown in FIG. 32D, where an exponential relationship is also observed. The weight percent loading was then normalized by the computed S-SSA, shown in FIG. 32E. The linear correlation between block distance and loading for β-Est and the plateauing effect for FdA reemerged with this analysis. This was also observed when the weight percent loading was normalized by the Delta SSA, FIG. 32F. This suggests that normalization purely by mass without accounting for geometry or changes in geometry can be misleading, particularly when the system is extended to more complex geometries such as IVRs.

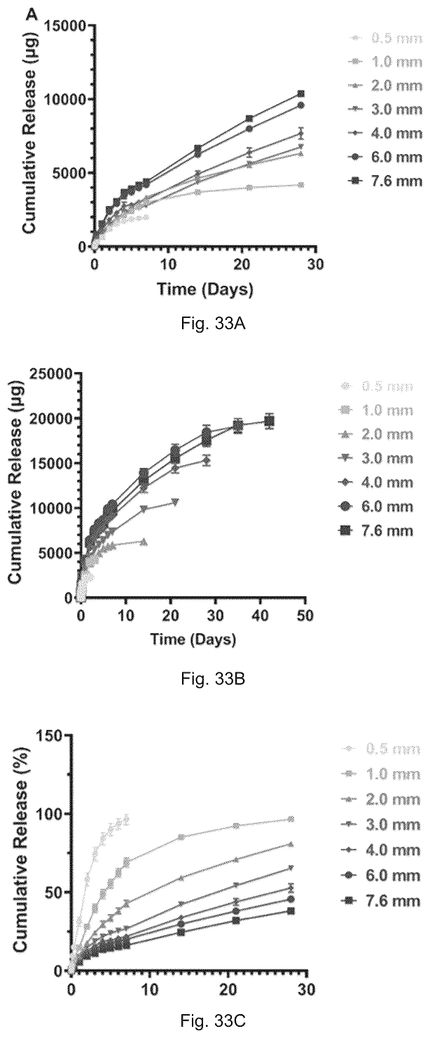

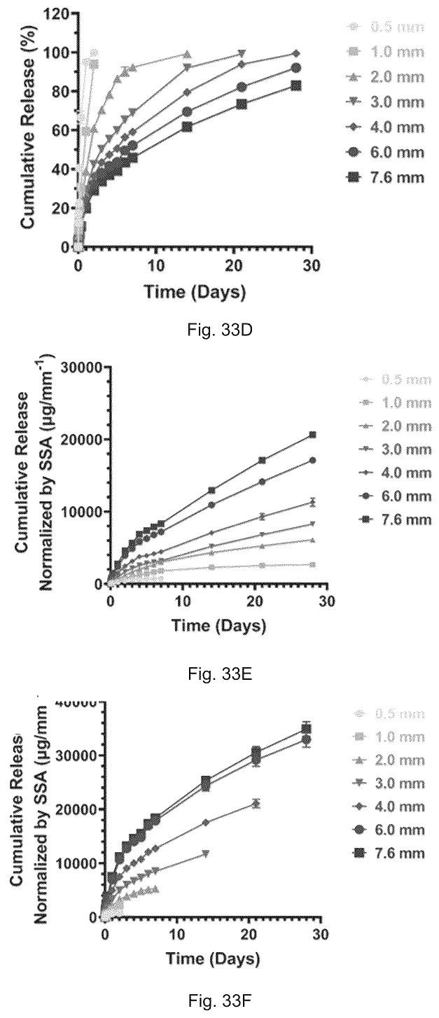

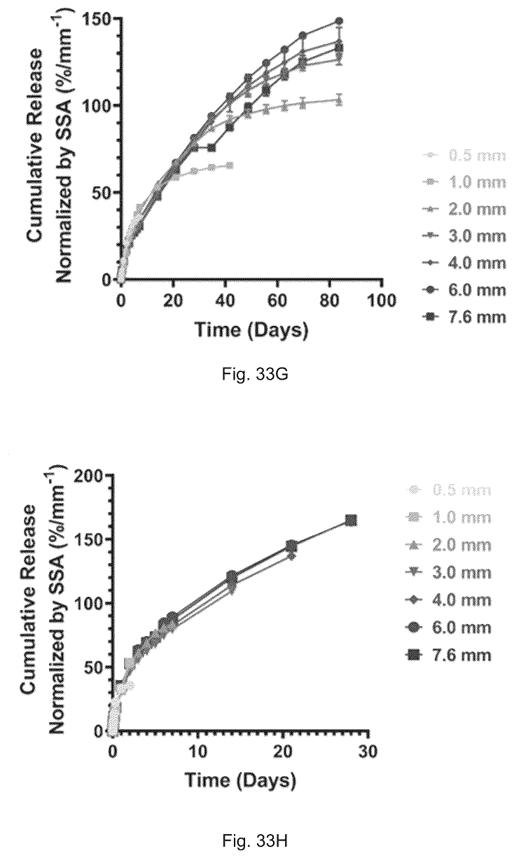

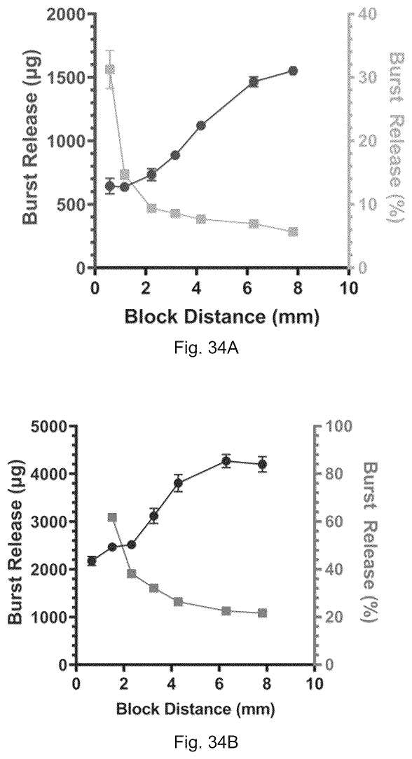

It has been demonstrated that post-loading results in a generally homogenous distribution within the SIL 30 polymer matrix, however as with all absorption methods, the possibility that API will release in one initial burst rather than a sustained delivery. In vitro release testing in simulated vaginal fluid (SVF) was conducted to assess the capacity of blocks loaded with a model drugs for sustained release. Cumulative release profiles in micrograms are shown for β-Est (FIG. 33A) and FdA (FIG. 33B) as a function of time and block distance. While β-Est demonstrates a longer release duration than FdA for all block types, this is to be expected given the hydrophobic nature of the model drug. All block types loaded with either β-Est or FdA demonstrate a linear and sustained release following an initial burst, which is consistent with the performance of other long-acting devices. These data support the observations made during the visualization that the drug has completely intercalated the matrix via absorption rather than remained on the surface. Had the uptake mechanism been largely driven by adsorption, then all blocks would have demonstrated a significant burst release within the first 24-48 hrs. Cumulative release profiles in relative percent are shown for β-Est (FIG. 33C) and FdA (FIG. 33D) as a function of time and block distance. Blocks were immersed in SVF until a plateau in release was observed. Blocks were then removed and fully extracted to capture any residual drug trapped in the polymer matrix. Cumulative percent releases therefore represent the total loaded amount in the block both from SVF and extracted. All blocks achieved near complete release. This can likely be attributed to the swelling behavior of the SIL 30 material in SVF, which allows for the moving front to solubilize the drug and diffuse out of the polymer matrix [35].

Initial analysis indicates the release rates appear to be driven by block distance however these blocks are not infinite planes and therefore additional geometric factors must be accounted for. For a drug eluting toroidal device, release is often normalized by the cross-sectional diameter of the torus, particularly when utilizing the mathematical relationships between diffusion coefficient (D_{f}) and drug solubility (Cₛ) established in the Higuchi Model [36, 37]. Therefore, as with analysis of total drug uptake, it was necessary to account for the geometry of the block given that they are not and were not intended to mimic infinite planes as seen in theoretical modeling [37]. Cumulative release was normalized per block type using the equations derived from FIG. 27G and the initial SSA values of the releasing blocks. The resulting normalized microgram release profiles can be seen in FIGS. 32E and F for β-Est and FdA, respectively. For β-Est, these profiles demonstrate a clear delineation between block distances and the normalized amount released per day appears to be driven by block distance. For the more hydrophilic FdA, the larger blocks of 6.0 and 7.6 mm appear to reach a threshold of release, while the smaller block distances delineate in a similar pattern as seen with β-Est release. While these blocks were immersed in the same concentration of post-loading solution, the drug uptake relative to the mass of the block was not consistent between blocks. To account for this, weight percent of drug was normalized by the SSA of the placebo SVF block swelling for β-Est (FIG. 32G) and FdA (FIG. 32H). For both model drugs, the cumulative profiles collapse into a single curve independent of block distance. This analysis reduces release to considering the competing interaction of the drug with the SIL 30 matrix and the surrounding SVF. Once geometry is accounted for, this value should be only dependent on the hydrophobicity of the drug, not the block distance. This is evidenced by the resulting slopes of the normalized curves. β-Est is significantly lower than FdA, as would be expected for comparison of a hydrophobic drug in a hydrophobic matrix release into an aqueous environment.

Cumulative release profiles were quantitatively assessed for β-Est (Table 20) and FdA (Table 21) by experimental diffusion block distance (as opposed to theoretical block type distance). Drug loading and weight percent loading from FIGS. 32A and B were tabulated for each model drug. Burst release was calculated as the cumulative release, both microgram and percent, within the first 24 hrs. Zero-order linear regressions were fitted to the release profiles and the respective coefficients of determination calculated. Calculation of release per day correlates with observations made from the cumulative release profiles, namely that the release per day is dependent on block distance (unnormalized).

TABLE 20
Release metrics of β-Est into SVF by experimental block distance. Drug loading amount and weight
percent tabulated as shown in FIG. 7A and B, respectively. Burst release quantified as the cumulative
release in the first 24 hrs. Zero order release determined from linear regression beginning at
Day 2. All values represent average and standard deviation of n = 4 samples per block type.

| | | | Burst Release | | | |
|---|---|---|---|---|---|---|
| Experimental | Drug | | Average | Average | Drug Release at Zero Order | |
| Distance | Loading | Wt. % | Per Block | Per Block | Mass (μg)/Day | Percent/Day |
| (mm) | (mg) | Loading | (μg) | (%) | (R²) | (R²) |
| 0.59 ± 0.03 | 2.06 ± 0.19 | 1.70 ± 0.09 | 644.4 ± 61.9 | 31.35 ± 2.12 | 126.68 ± 10.25 | 6.17 ± 0.52 |
| | | | | | (0.864) | (0.864) |
| 1.17 ± 0.05 | 4.33 ± 0.15 | 1.75 ± 0.05 | 636.9 ± 33.4 | 14.70 ± 1.13 | 192.13 ± 8.10 | 4.45 ± 0.09 |
| | | | | | (0.874) | (0.864) |
| 2.23 ± 0.07 | 7.80 ± 0.15 | 1.74 ± 0.03 | 733.3 ± 47.9 | 9.24 ± 0.84 | 194.66 ± 6.63 | 2.18 ± 0.03 |
| | | | | | (0.942) | (0.942) |
| 3.16 ± 0.06 | 10.34 ± 0.19 | 1.53 ± 0.03 | 888.0 ± 36.4 | 8.59 ± 0.35 | 197.57 ± 3.57 | 1.91 ± 0.03 |
| | | | | | (0.993) | (0.993) |
| 4.18 ± 0.04 | 14.55 ± 0.38 | 1.54 ± 0.04 | 1120.4 ± 18.0 | 7.70 ± 0.12 | 219.83 ± 13.62 | 1.51 ± 0.09 |
| | | | | | (0.994) | (0.994) |
| 6.25 ± 0.08 | 21.03 ± 0.68 | 1.45 ± 0.11 | 1466.1 ± 39.7 | 6.97 ± 0.19 | 269.93 ± 7.99 | 1.28 ± 0.04 |
| | | | | | (0.993) | (0.993) |
| 7.80 ± 0.09 | 27.22 ± 1.48 | 1.43 ± 0.06 | 1552.1 ± 25.3 | 5.70 ± 0.09 | 296.76 ± 7.04 | 1.09 ± 0.03 |
| | | | | | (0.994) | (0.994) |

TABLE 21
Release metrics of FdA into SVF by experimental block distance. Drug loading amount and weight
percent tabulated as shown in FIG. 7A and B, respectively. Burst release quantified as the cumulative
release in the first 24 hrs. Zero order release determined from linear regression beginning at
Day 2. All values represent average and standard deviation of n = 4 samples per block type.

| | | | Burst Release | | | |
|---|---|---|---|---|---|---|
| Experimental | Drug | | Average | Average | Drug Release at Zero Order | |
| Distance | Loading | Wt. % | Per Block | Per Block | Mass (μg)/Day | Percent/Day |
| (mm) | (mg) | Loading | (μg) | (%) | (R²) | (R²) |
| 0.65 ± 0.01 | 2.07 ± 0.06 | 1.71 ± 0.07 | 2174.03 ± 95.51 | 95.28 ± 0.45 | — | — |
| 1.51 ± 0.01 | 4.07 ± 0.19 | 1.60 ± 0.07 | 2515.91 ± 24.25 | 61.81 ± 0.60 | — | — |
| 2.32 ± 0.02 | 6.46 ± 0.17 | 1.46 ± 0.04 | 2465.01 ± 31.59 | 38.86 ± 1.02 | 406.25 ± 19.80 | 6.40 ± 0.20 |
| | | | | | (0.947) | (0.947) |
| 3.26 ± 0.03 | 9.68 ± 0.70 | 1.52 ± 0.11 | 3116.23 ± 158.70 | 32.18 ± 1.64 | 423.43 ± 17.17 | 3.94 ± 0.06 |
| | | | | | (0.974) | (0.974) |
| 4.28 ± 0.04 | 14.42 ± 1.21 | 1.62 ± 0.14 | 3804.36 ± 180.22 | 26.39 ± 1.25 | 450.51 ± 16.45 | 2.93 ± 0.03 |
| | | | | | (0.975) | (0.975) |
| 6.29 ± 0.04 | 18.95 ± 1.28 | 1.43 ± 0.10 | 4267.89 ± 138.39 | 22.52 ± 0.73 | 453.56 ± 20.13 | 2.26 ± 0.01 |
| | | | | | (0.972) | (0.972) |
| 7.81 ± 0.08 | 19.38 ± 0.54 | 1.19 ± 0.03 | 4200.47 ± 162.07 | 21.68 ± 0.84 | 454.52 ± 20.34 | 1.86 ± 0.04 |
| | | | | | (0.975) | (0.975) |

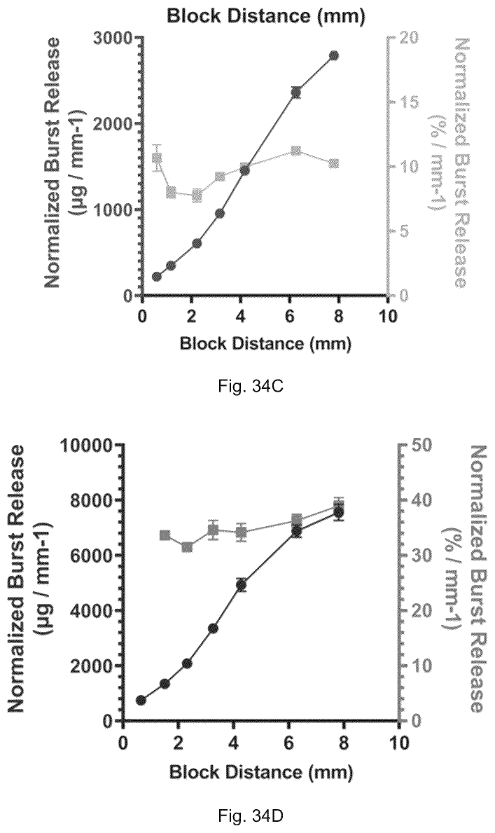

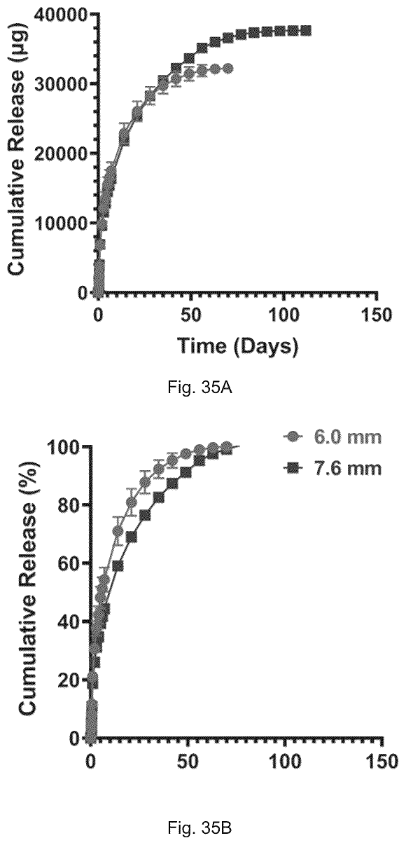

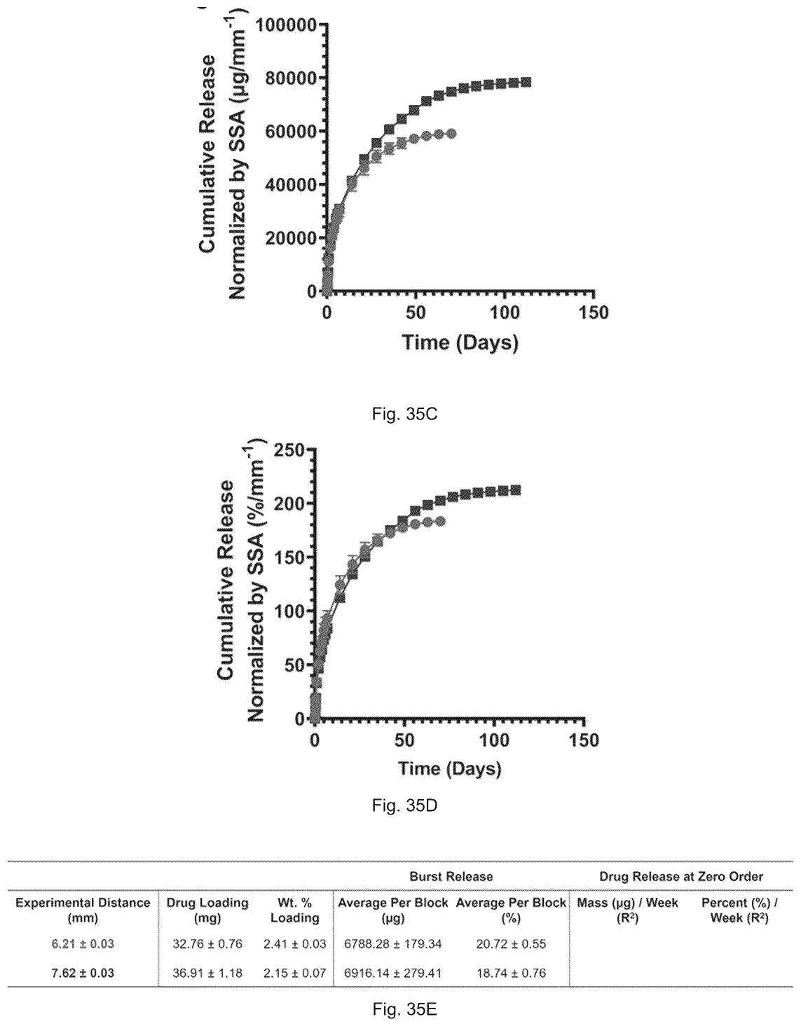

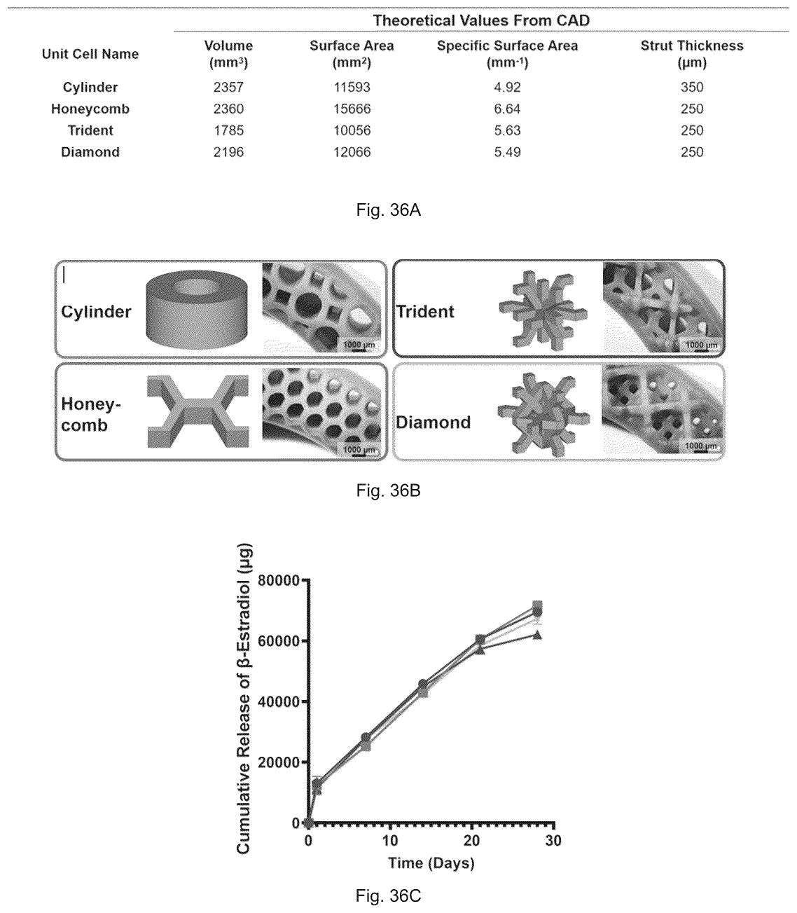

A defining feature of any drug-delivery device is the percent burst release and for long-acting devices, it is necessary that this value is low to sustain delivery performance. Blocks loaded with model drugs were assessed for burst release, defined as amount eluted from the device in the first 24 hrs. Microgram and percent burst for β-Est and FdA are shown in FIGS. 34A and B, respectively. For both model drugs, there is an S-curve relationship between amount burst release and experimental block distance. When accounting for total loaded amounts, percent burst release, conversely shows an exponential relationship where the smaller block distances release a greater percent of drug compared to larger block distances. For the hydrophobic β-Est, the exponential plateaus around 6% whereas the hydrophilic FdA plateaus at 22%. This makes intuitive sense as an object with a large specific surface area has a larger area exposed to release media and therefore should experience a larger burst release when compared to an object with a low SSA.

The role of specific surface area in the behavior of the blocks has been previously demonstrated as important, and therefore must be accounted for in the swollen SVF state. This was done for β-Est (FIG. 34C) and FdA (FIG. 34D) for microgram and percent release. For both model drugs, the original S-curve in microgram burst release because more linear once the geometry of the part was accounted for, as seen with the cumulative release rates. For percent burst, normalization by SSA resulted in relatively stable across all block distances. For β-Est, normalized percent stabilized around 10% and FdA around 40%.

TABLE 22
Drug loading metrics of EFdA and FdA in 6.0 and 7.6 mm SIL 30 block types. All
values represent average and standard deviation of n = 4 samples per block type.

| | Distance | | | | Normalized | | |
|---|---|---|---|---|---|---|---|
| | Diffusion | | Loading | Swollen SSA | Wt. % by | | Normalized |
| Drug | (mm) | Drug (mg) | (Wt. %) | (S-SSA) | S-SSA | Delta SSA | Delta SSA |
| EFdA | 6.21 ± 0.03 | 32.76 ± 0.76 | 2.41 ± 0.06 | 0.40 ± 0.003 | 81.4 ± 2.26 | −0.22 ± 0.003 | −150.63 ± 4.00 |
| | 7.62 ± 0.03 | 36.91 ± 1.18 | 2.15 ± 0.07 | 0.37 ± 0.002 | 99.91 ± 3.67 | −0.19 ± 0.003 | −195.95 ± 4.41 |
| FdA | 6.29 ± 0.04 | 18.95 ± 1.28 | 1.43 ± 0.10 | 0.40 ± 0.003 | 47.53 ± 3.10 | −0.21 ± 0.003 | −91.19 ± 7.35 |
| | 7.81 ± 0.08 | 19.38 ± 0.54 | 1.19 ± 0.03 | 0.37 ± 0.001 | 52.82 ± 1.47 | −0.19 ± 0.002 | −103.29 ± 2.04 |

### Example 18

### Translation into Geometrically Complex IVRs

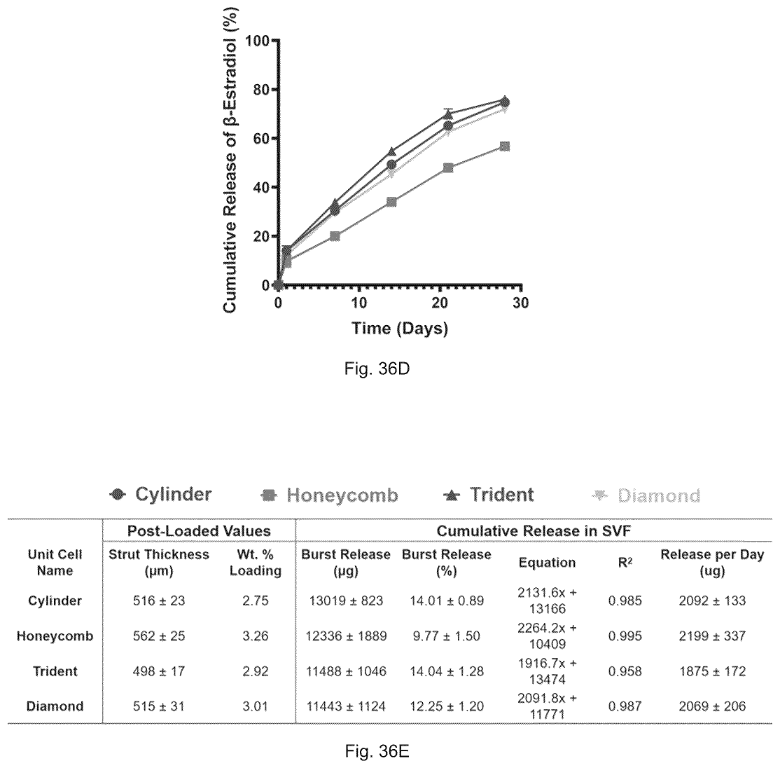

To demonstrate the utility of post-fabrication drug absorption, and by way of example only and not limitation, the post-loading method was translated into a medical device using the model drug β-Estradiol (β-Est). As previously described, geometrically complex IVRs represent an attractive next-generation medical device for sustained drug delivery [33]. The incorporation of design complexity via CAD has been proposed to enable the control of release characteristics such as release rate and duration. Therefore, to assess the value of controlling for diffusion distance via geometric complexity, four unit cells were developed and explored. The theoretical values associated with the resulting geometrically complex IVRs are shown in FIG. 36A and range volume, surface area, and SSA. The purpose of this ring series was to explore release of a model drug from different designs that result in a similar strut thickness. To that end, it should be noted that the input CAD strut thickness was specifically not held constant as it has been previously demonstrated that fabrication in SIL 30 over-prints the wall thickness in a way that is dependent both on input geometry and part placement [33]. Therefore, the input strut thickness was selected such that the final fabricated distance would be approximately 500 μm. Images of the CAD unit cell architectures and of the resulting rings fabricated in SIL 30 are shown in FIG. 36B. It should be noted that these designs are not intended for clinical use but are rather an exercise in design parameter control.

Rings were loaded with a model drug, β-Est, at 22 mg/mL in methanol for 24 hrs. Methanol was selected for ring exposure given the higher boiling point relative to acetone and thus, a slower evaporation from the SIL 30 matrix. There is a risk of interior architecture disruption if the swelling and drying of the polymer matrix is too rapid. Therefore, the selection of MeOH was a precautionary step and as with previous results, the post-loading method should be investigated for extension to other Class II/III solvents such as ethanol. IVRs were tested for in vitro release in SVF medium. The cumulative release profiles are shown in FIGS. 36C and D. As with the blocks, the cumulative microgram release per day profiles collapsed into a similar release per day. The cumulative percent release profiles are similar and trend according to the experimentally determined strut thickness, tabulated in FIG. 36E. The microgram release per day was found to be approximately 2 mg/day independent of unit cell design. These data suggest that release rate, as shown in the analysis of the blocks, is driven by diffusion distance. This could be attributed to the differences in release from complex interstitial void volumes compared to release from flat planar surfaces.

### Example 19

### Pre-Loading vs. Post-Loading of β-Estradiol in 3D Printed Intravaginal Rings

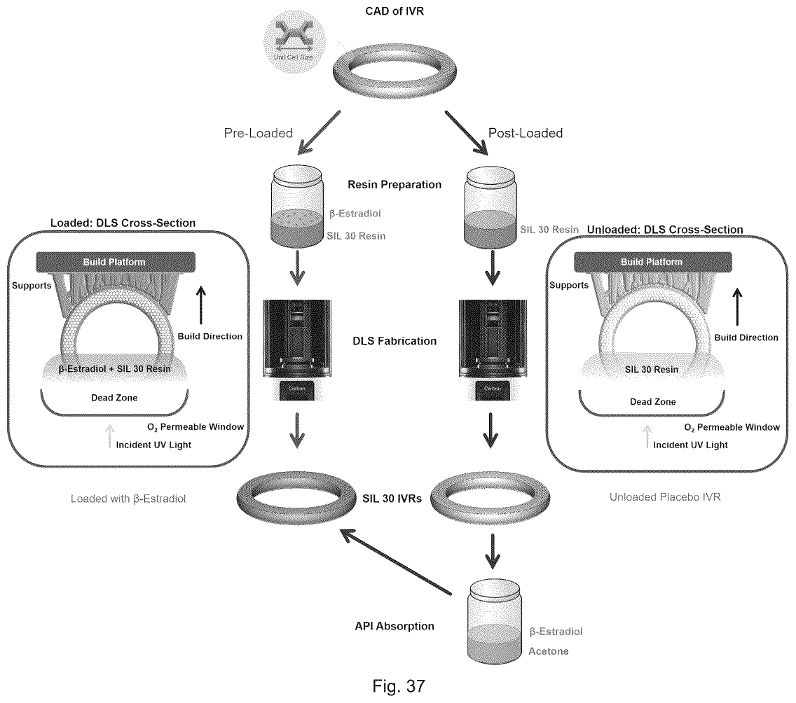

Rings were fabricated on an M1 DLS (Carbon, Inc.) 3D-printer. Rings were uploaded to the Carbon UI, orientated vertically and supported with approximately 40 supports per ring. Rings were fabricated in a silicone-based polyurethane resin (SIL 30). This is a two-part resin that was dispensed immediately prior to printing using the Carbon-supplied static mixer. Each print consisted of 16 rings, vertically oriented, fabricated using approximately 150 mL of SIL 30. Carbon's pre-optimized and standardized exposure parameters for SIL 30 were used and yielded a fabrication time of 3 hr. 9 min. Rings were removed with a razor following fabrication and the supports separated. Rings were exposed to two 30 s cycles of approximately 200 mL of IPA per ring using a previously optimized cleaning process. SIL 30 rings were allowed to air dry for 45 min and then placed in a programmable oven for 8 hr. at 120° C., as prescribed by Carbon (FIG. 37).

In the preloaded IVRs, a pre-determined amount of β-estradiol was mixed in with the resin prior to the printing process. An aliquot of the resin was used to determine analytical drug concentration in the resin. Placebo IVRs were printed using identical parameters as the drug loaded IVRs. Masses and dimensions were recorded before drug incorporation using absorption. A pre-determined amount of β-estradiol was dissolved in acetone and the placebo IVRs were placed in this solution for 24 hours. These were later removed and dried in a fume hood for 24 hours. Once the mass has plateaued, the dimensions and final masses were recorded.

The concentration of β-estradiol+acetone solution was determined by the use of the loading equation. There is a linear relationship between the API-Solvent concentration and the amount of API incorporated in the SIL30 3D printed IVR. This linear relationship has been used to derive the loading equation. The maximum loading achieved is 13.6 mg β-estradiol/1000 mg of SIL30 3D CLIP printed IVR, using acetone as the loading solvent, and the loading duration of 24 hours. The IVRs were removed after 24 hours and dried in a fume hood until IVR mass has plateaued; the dimensions and final masses were recorded.

### Example 20

### In Vitro Release of β-Estradiol From the 3D Printed IVRs

Studies to measure in vitro release of β-estradiol into a simulated vaginal fluid (SVF) were carried out on 3D-IVRs (n=4). The SVF consisted of 25 mM sodium acetate buffer (pH 4.2) plus 2% Solutol (Kolliphor HS 15). The IVRs were placed in straight-sided glass jars containing 200 mL SVF at 37±2° C. 1 ml aliquots of the release medium were removed at specified time intervals and complete media changes were carried out to maintain sink conditions. The concentration of β-Estradiol in the aliquots was determined using an Agilent 1260 HPLC with a Diode Array Detector, on an Inertsil ODS-3 column (4.6×150 mm, 5 μm) maintained at 40° C., with a flow rate of 1.0 mL/min, 25 ul sample injection, and an acetonitrile/water mobile phase, each modified with 0.1% trifluoroacetic acid. A gradient method was utilized to achieve separation (0-20 min: 5%-100% acetonitrile; 20-22 min: 100% acetonitrile; 23-25 min: 5% acetonitrile). β-Estradiol was eluted at 13.8 min. Data collected at 280 nm was computed using Chemstation software, and concentrations were derived from a calibration curve generated using β-Estradiol standards prepared in 100% acetonitrile (250 ug/ml-61 ng/ml).

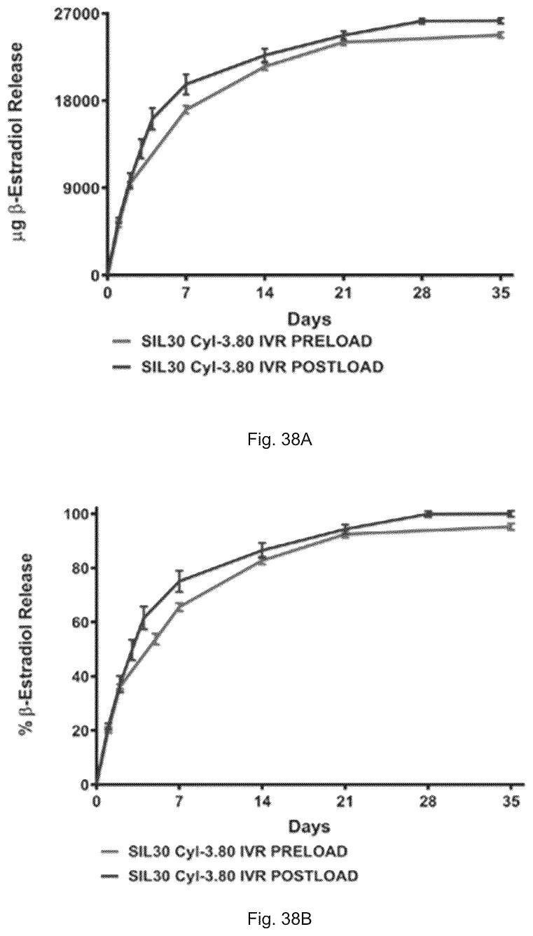

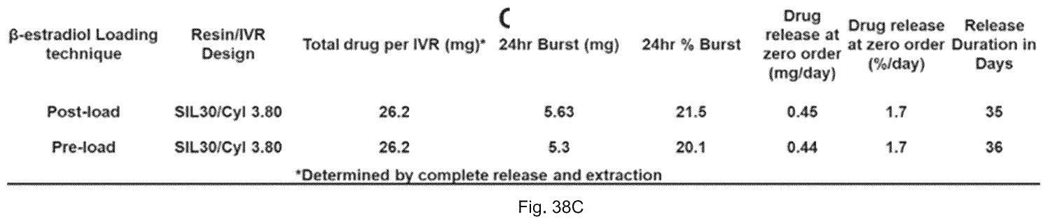

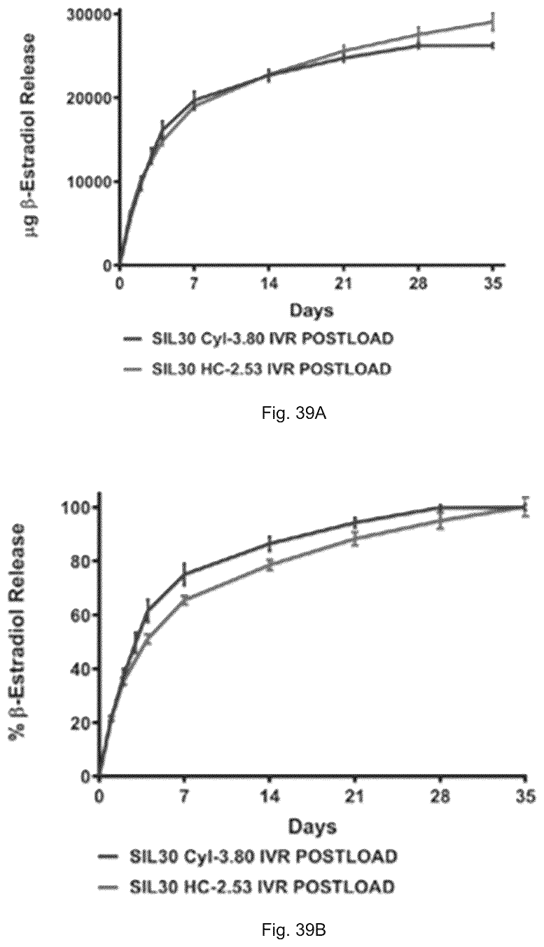

The preload process most closely resembles the matrix IVR design used in many marketed IVRs. Keeping the API loading the same in preload and postload IVRs, we did a head-to-head comparison of the two different loading techniques. A zero-order release kinetics pattern was observed in both groups, as can be observed in FIG. 38. burst and per day mg release of estradiol are almost identical. Also important to note was the 100% release of API from both these groups with no API trapped in the device.

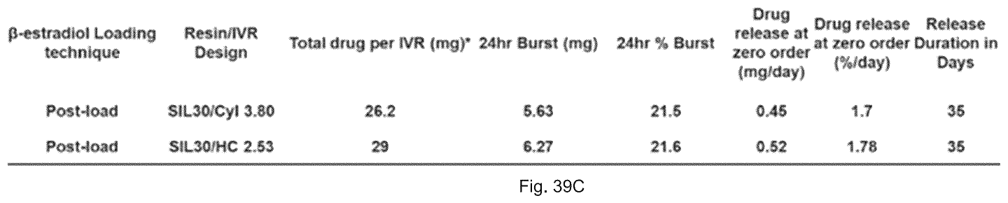

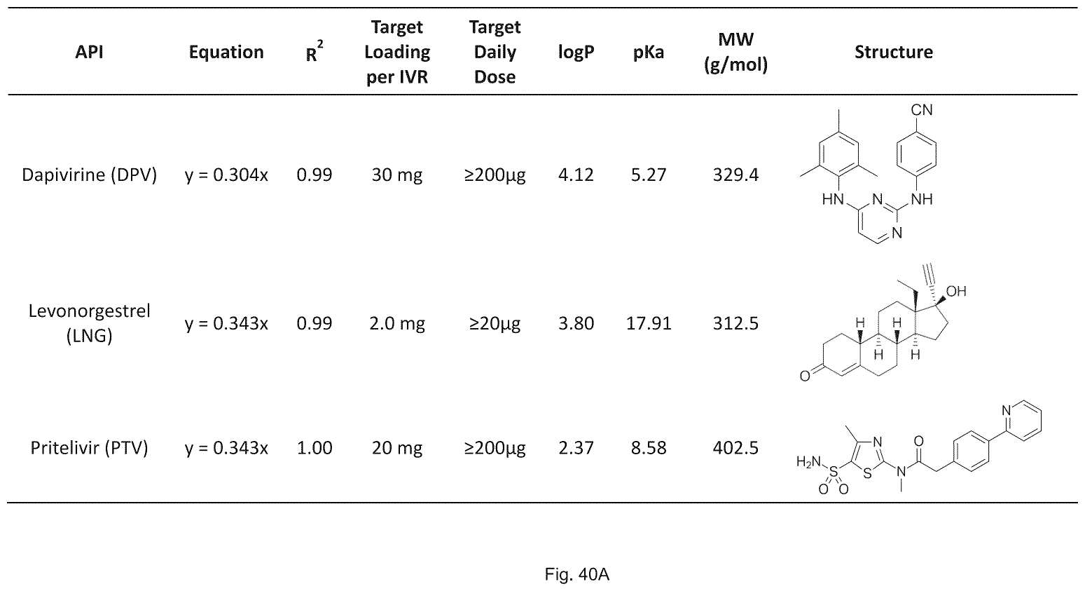

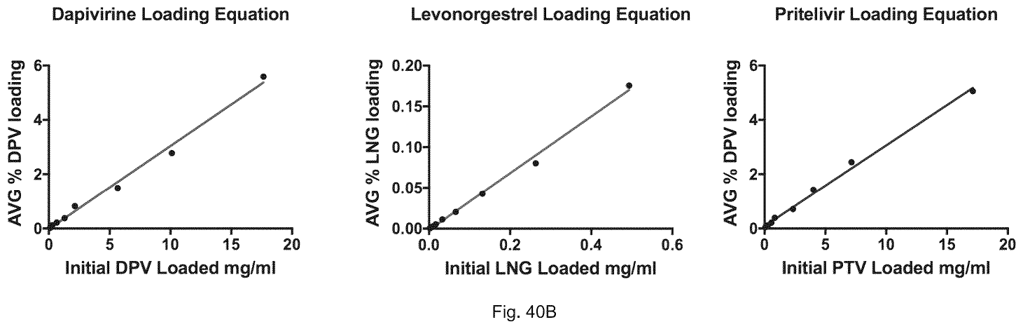

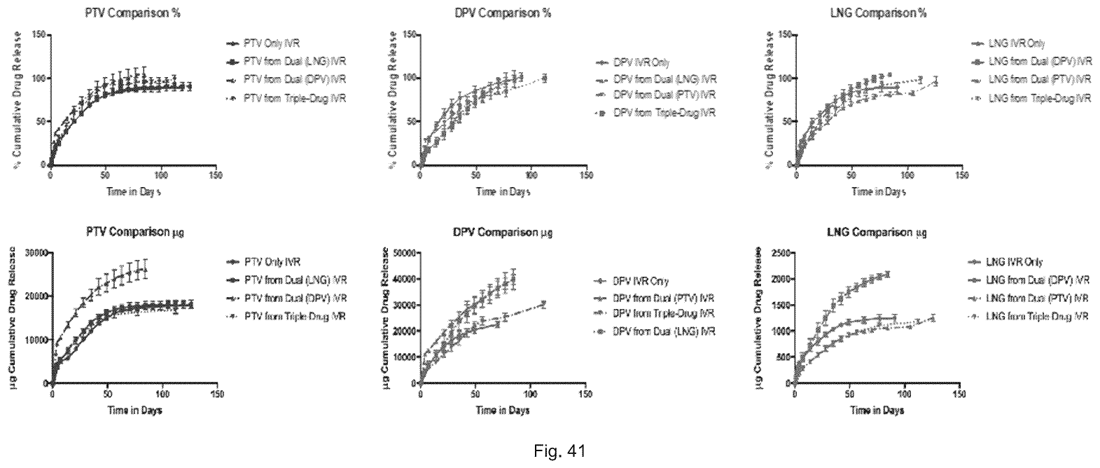

Having established that both these methods provide the same in-vitro release profiles, we wanted to further investigate the post loading method. The Cyl-3.80 and HC-2.53 IVR designs were post-loaded with β-estradiol, and their release profile is shown in FIG. 6. When post-loaded with the same drug amount (26-29 mg/IVR), the two IVR designs exhibited the same release profiles including burst release within the first 24 h and daily release rate at zero order kinetics (FIG. 39). For both designs, complete release (100%) of β-estradiol was achieved. More importantly, the two designs have the same strut thickness, furthering the case that release kinetics is dictated by design parameters, strut thickness being an important design element in fine-tuning release kinetics.

### Example 21

### Developing Multi-Purpose Technology (MPT) IVRs for Sustained Delivery of Multiple Drugs

MPT IVRs for prevention of HIV, HSV-2 and unplanned pregnancy are being developed using our 3D printed IVR technology.

Three (3) APIs were loaded in a single IVR using our post-loading method. First, loading equations were developed and validated for each API (FIG. 40). All APIs were loaded as single drug or in combination with one or two other APIs to compare their release kinetics when loaded alone vs. in combination with other APIs. Our results showed that all APIs exhibited similar release kinetics when loaded alone (single drug IVR) or in combination with one (dual drug IVR) or two (triple drug IVR) demonstrating that no drug-drug interaction effect resulted in combination drug IVRs (FIG. 41).

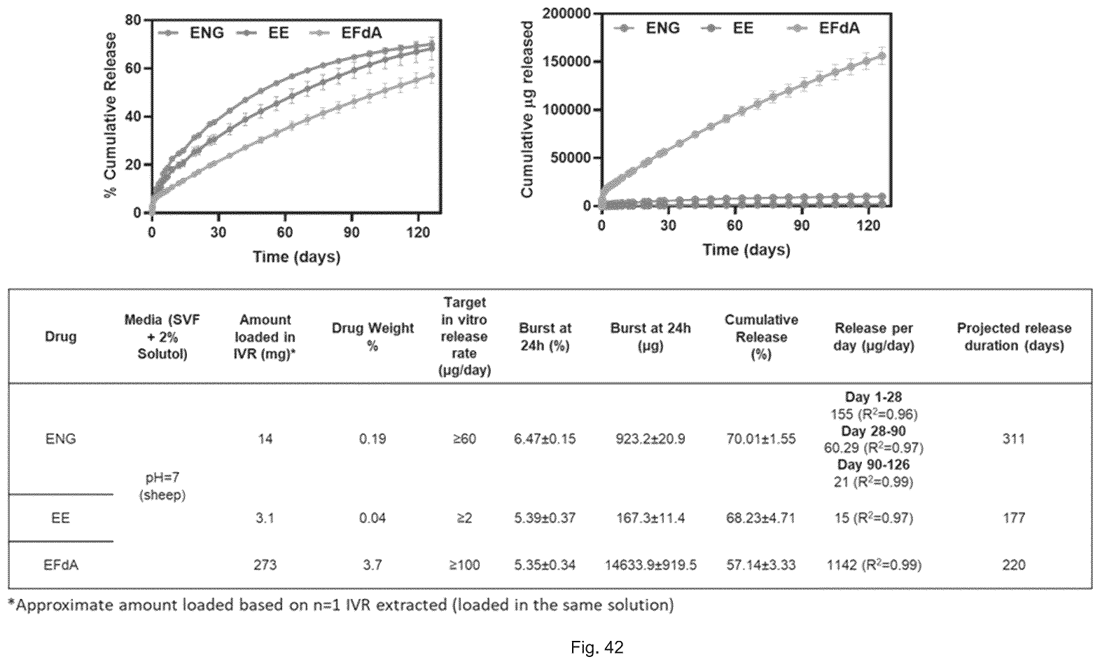

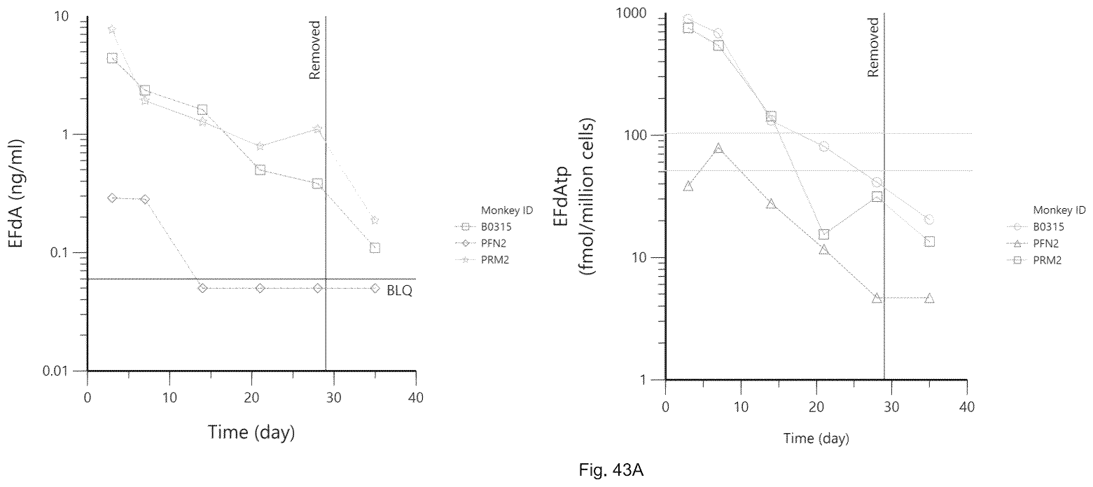

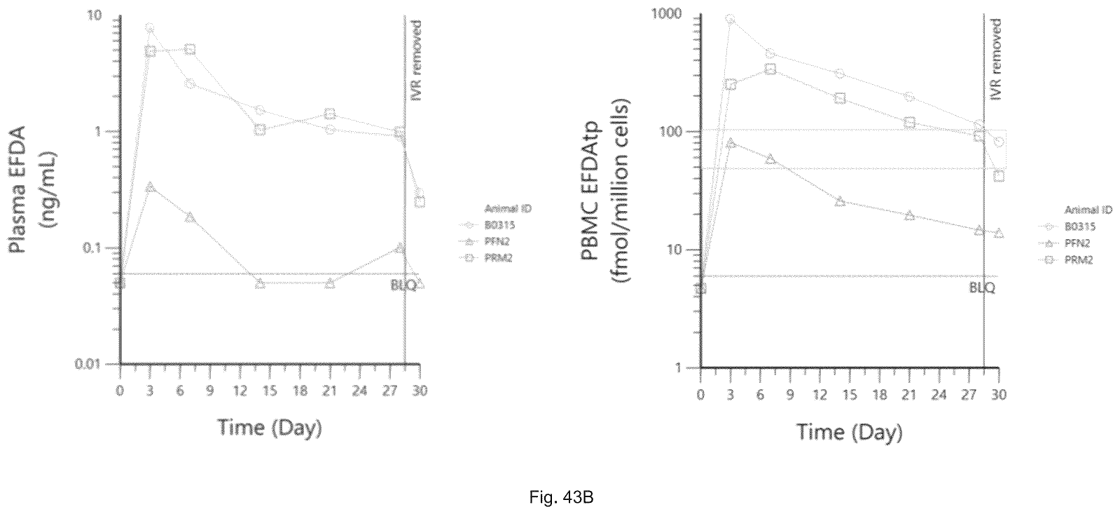

Another MPT IVR for prevention of HIV and unplanned pregnancy has been developed with an anti-HIV drug (Islatravir, EFdA) and the NuvaRing contraceptive combination (etonogestrel/ethinylestradiol, ENG/EE). All drugs were successfully loaded in a single IVR using our post-loading method to achieve target loading for each drug and target release rates for over 4 months (last time point analyzed) (FIG. 42).

### Example 22

### In Vivo Macaque Pharmacokinetic (PK) Studies

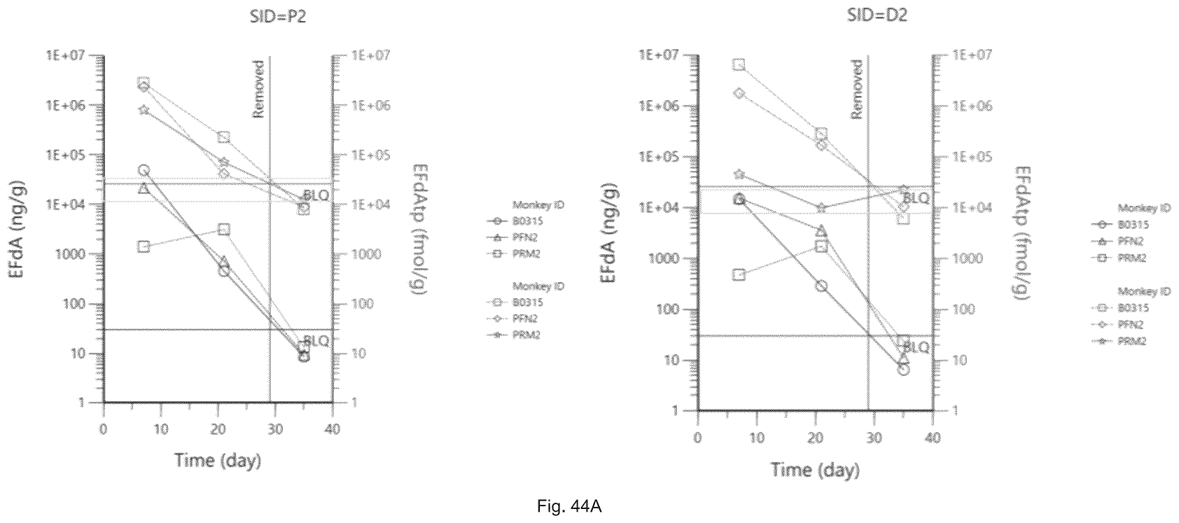

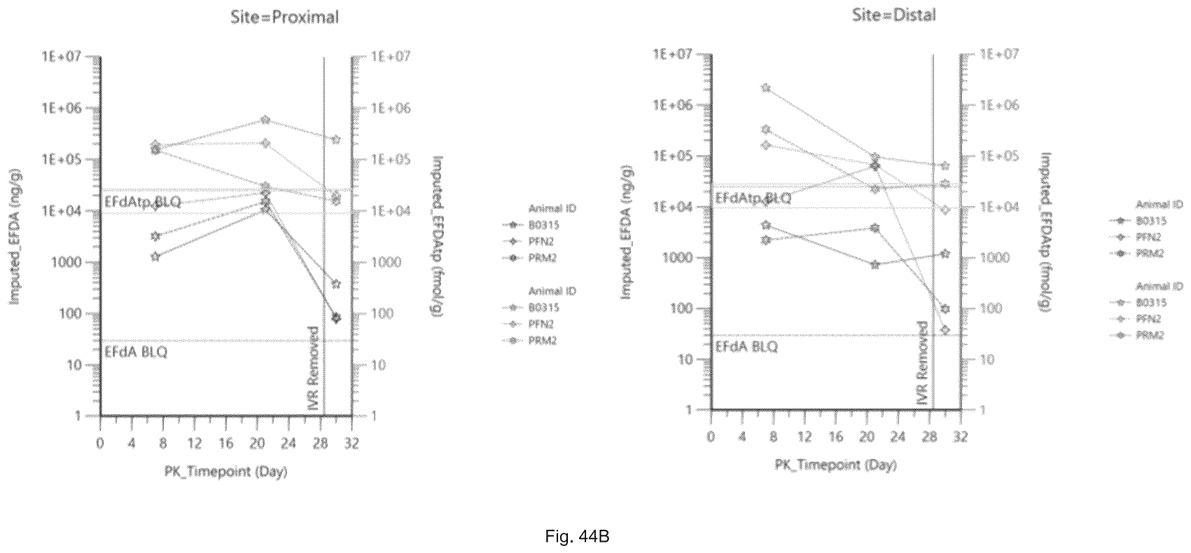

Macaque size (25 mm OD) 3D printed IVRs post-loaded with EFdA at 45 mg/IVR or 62 mg/IVR were administered to female pigtailed macaques and plasma, PBMCs, vagina and rectal fluids, and vaginal and rectal biopsies were collected over 28 days and analyzed for EFdA concentration and EFdA-TP (triphosphorilated EFdA, EFdA-TP) in PBMCs. Results showed that PK levels were dose dependent with higher levels achieved with the 62 mg EFdA IVR compared to the 45 mg EFdA IVR. More importantly EFdA-TP levels in PBMCs and EFdA levels in tissue and fluid samples were above the known efficacious levels based on preclinical studies in NHP and human clinical trials with oral EFdA (FIG. 43-44). Collectively, these data demonstrate the effectiveness of the post-loading process in achieving target drug loading of one or multiple drugs in a single IVR and achieving sustained drug release both in vitro and in vivo.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

1. Paolini, M. S., et al., Polymers for extended-release administration. Biomed Microdevices, 2019. 21 (2): p. 45.

2. Liechty, W. B., et al., Polymers for drug delivery systems. Annu Rev Chem Biomol Eng, 2010. 1: p. 149-73.

3. Grund, S., M. Bauer, and D. Fischer, Polymers in Drug Delivery-State of the Art and Future Trends. Advanced Engineering Materials, 2011. 13 (3): p. B61-B87.

4. Lowinger, M. B., et al., Sustained Release Drug Delivery Applications of Polyurethanes. Pharmaceutics, 2018. 10 (2).

5. Bhise, V. B. K. M. S. N. I. K., Biodegradable Polymers—A Review. Indian Journal of Pharmaceutical Sciences, 2007. 69 (5): p. 616-625.

6. Chou, S. F., D. Carson, and K. A. Woodrow, Current strategies for sustaining drug release from electrospun nanofibers. J Control Release, 2015. 220 (Pt B): p. 584-91.

7. Maturavongsadit, P., et al., Biodegradable polymeric solid implants for ultra-long-acting delivery of single or multiple antiretroviral drugs. Int J Pharm, 2021. 605: p. 120844.

8. Benhabbour, S. R., et al., Ultra-long-acting tunable biodegradable and removable controlled release implants for drug delivery. Nat Commun, 2019. 10 (1): p. 4324.

9. Mondal, S., S. Das, and A. K. Nandi, A review on recent advances in polymer and peptide hydrogels. Soft Matter, 2020. 16 (6): p. 1404-1454.

10. Monzón, M. D., et al., Standardization in additive manufacturing: activities carried out by international organizations and projects. The International Journal of Advanced Manufacturing Technology, 2014. 76 (5-8): p. 1111-1121.

11. Zhou, L. Y., J. Fu, and Y. He, A Review of 3D Printing Technologies for Soft Polymer Materials. Advanced Functional Materials, 2020. 30 (28).

12. Ligon, S. C., et al., Polymers for 3D Printing and Customized Additive Manufacturing. Chem Rev, 2017. 117 (15): p. 10212-10290.

13. Beg, S., et al., 3D printing for drug delivery and biomedical applications. Drug Discov Today, 2020. 25 (9): p. 1668-1681.

14. Xu, X., et al., Vat photopolymerization 3D printing for advanced drug delivery and medical device applications. J Control Release, 2021. 329: p. 743-757.

15. Yan, Q., et al., A Review of 3D Printing Technology for Medical Applications. Engineering, 2018. 4 (5): p. 729-742.

16. Sadia, M., et al., Channelled tablets: An innovative approach to accelerating drug release from 3D printed tablets. J Control Release, 2018. 269: p. 355-363.

17. Krkobabic, M., et al., Hydrophilic excipients in digital light processing (DLP) printing of sustained release tablets: Impact on internal structure and drug dissolution rate. Int J Pharm, 2019. 572: p. 118790.

18. Chai, X., et al., Fused Deposition Modeling (FDM) 3D Printed Tablets for Intragastric Floating Delivery of Domperidone. Sci Rep, 2017. 7 (1): p. 2829.

19. Fina, F., et al., 3D printing of drug-loaded gyroid lattices using selective laser sintering. Int J Pharm, 2018. 547 (1-2): p. 44-52.

20. Qiao, X., et al., E-Jet 3D-Printed Scaffolds as Sustained Multi-Drug Delivery Vehicles in Breast Cancer Therapy. Pharm Res, 2019. 36 (12): p. 182.

21. Karakurt, I., et al., Stereolithography (SLA) 3D printing of ascorbic acid loaded hydrogels: A controlled release study. Int J Pharm, 2020. 584: p. 119428.

22. Taufik, M., Role of build orientation in layered manufacturing: a review. Int. J. Manufacturing Technology and Management, 2013. 17 (1/2/3): p. 47-72.

23. Goh, G. D., et al., Process-Structure-Properties in Polymer Additive Manufacturing via Material Extrusion: A Review. Critical Reviews in Solid State and Materials Sciences, 2019. 45 (2): p. 113-133.

24. To, L. C. A., Part-Scale Build Orientation Optimization for Minimizing Residual Stress and Support Volume for Metal Additive Manufacturing: Theory and Experimental Validation. Computer-Aided Design, 2019. 113: p. 1-23.

25. Gao, W., et al., The status, challenges, and future of additive manufacturing in engineering. Computer-Aided Design, 2015. 69: p. 65-89.

26. Abdulhameed, O., et al., Additive manufacturing: Challenges, trends, and applications. Advances in Mechanical Engineering, 2019. 11 (2).

27 T. Samulski; Joseph M. DeSimone, et al., Continuous liquid interface production of 3D objects. Science, 2015. 347 (6228): p. 1349-1352.

28. Janusziewicz, R., et al., Layerless fabrication with continuous liquid interface production. Proc Natl Acad Sci U S A, 2016. 113 (42): p. 11703-11708.

29. Rolland, J. P. J., Rethinking digital manufacturing with polymers. Science, 2017. 358 (6369): p. 1384-1385.

30. Xinyu Gu, J. P., Jason P. Rolland, Methods of making three dimensional objects from dual cure resins with supported second cure, I. Carbon3D, Editor. 2018: United States of America.

31. Johnson, A. R., et al., Single-Step Fabrication of Computationally Designed Microneedles by Continuous Liquid Interface Production. PLOS One, 2016. 11 (9): p. e0162518.

32. Bloomquist, C. J., et al., Controlling release from 3D printed medical devices using CLIP and drug-loaded liquid resins. J Control Release, 2018. 278: p. 9-23.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of treating a subject, wherein the method comprises:
providing a subject in need of treatment, prevention or a diagnostic;
providing a medical device comprising a polymer matrix and an active pharmaceutical ingredient (API), wherein the API is loaded into the polymer matrix by adsorption and/or swelling after fabrication of the polymer matrix, wherein the medical device achieves release kinetics in a range of about one day to about 360 days; and
administering, placing and/or applying the medical device to or in the subject in need of treatment, prevention or diagnostic.

2. The method of claim 1, wherein the medical device comprises an intravaginal ring (IVR), wherein the IVR comprises one or more APIs.

3. The method of claim 2, further comprising a release rate controlling additive.

4. The method of claim 1, wherein the medical device comprises an intravaginal ring (IVR), wherein the IVR comprises a release rate controlling polymeric membrane.

5. The method of claim 1, wherein release kinetics of the one or more APIs are in a range of about one day to about 360 days.

6. The method of claim 1, wherein the one or more APIs comprises a therapeutic compound selected from an antiviral, antiretroviral, microbicide, contraceptive, antibiotic, hormone, pre-exposure prophylaxis, small molecule drug, macromolecule drug, biopharmaceutical, chemotherapeutic, monoclonal antibody, protein, peptide, diagnostic marker, other pharmaceutical compound, and combinations thereof.

7. The method of claim 1, wherein the subject in need of treatment is in need of HIV pre-exposure prophylaxis (PrEP), HIV treatment, contraception, and/or prevention of sexually transmitted diseases (STDs).

8. The method of claim 1, wherein the subject in need of treatment is in need of treatment of infertility, hormone replacement, or gynecology oncology.

9. The method of claim 1, wherein the subject in need of treatment is a female human subject or transgender.

10. The method of claim 1, wherein the release kinetics comprise a substantially sustained release for at least about 30 days or more.

11. The method of claim 10, wherein the release kinetics comprise a substantially sustained release for at least about 60 days or more.

12. The method of claim 10, wherein the release kinetics comprise a substantially sustained release, optionally for at least about 90 days or more, optionally for at least about 120 days or more.

13. The method of claim 1, wherein a total loaded amount of API is tuned by an initial concentration of the loading solution comprising the API.

14. The method of claim 1, wherein a degree of crosslinking of the polymer matrix is substantially proportional to a degree of swelling, wherein the degree of crosslinking defines an accessibility of a micro space within the polymer matrix.

15. The method of claim 14, wherein the degree of swelling is substantially directly proportional to the degree of API loading.

16. The method of claim 14, wherein the degree of swelling of the polymer matrix substantially increases with increasing diffusion distance in the polymer matrix.

17. The method of claim 1, wherein the medical device comprises more than one API.

18. The method of claim 1, wherein extractables and/or leachables have been removed from the polymer matrix of the device.

19. A method of treating a subject, the method comprising:
providing a subject in need of treatment, prevention or a diagnostic;
providing a medical device comprising a polymer matrix and an active pharmaceutical ingredient (API), wherein the API is loaded into the polymer matrix by adsorption and/or swelling after fabrication of the polymer matrix, wherein the medical device achieves release kinetics in a range of about one day to about 360 days, wherein a release rate of the API from the polymer matrix is controlled by an interaction between the API and polymer matrix, and/or an interaction between the API and a surrounding environment, wherein the surrounding environment comprises one or more of:
swelling of polymer matrix parts that impact accessibility to API;
a surface area of the device that impacts accessibility to API;
another API or release rate controlling additive that impacts accessibility to API;
a polymeric membrane that surrounds the device and impacts accessibility to API; and
an initial loaded concentration of API and changes thereto as API is released; and
administering, placing and/or applying the medical device to or in the subject in need of treatment, prevention or diagnostic.

20. The method of claim 19, wherein the release kinetics comprise a substantially sustained release for at least about 30 days or more.
